(19) United States
(12) Patent Application Publication
(10) Pub. No.: US 2022/0025322 A1
(43) Pub. Date: Jan. 27, 2022

(54) COMPOSITIONS AND METHODS FOR PRINTING THREE-DIMENSIONAL STRUCTURES CORRESPONDING TO BIOLOGICAL MATERIAL

(71) Applicant: Prellis Biologics, Inc., Hayward, CA (US)

(72) Inventors: Melanie P. MATHEU, San Francisco, CA (US); Karl Johan Torbjorn BORGLIN, Goteborg (SE)

(21) Appl. No.: 17/318,947

(22) Filed: May 12, 2021

Related U.S. Application Data
(63) Continuation of application No. PCT/US2019/061035, filed on Nov. 12, 2019.
(60) Provisional application No. 62/760,766, filed on Nov. 13, 2018.

(51) Int. Cl. C12N 5/00; C12N 5/071; B29C 64/124; B33Y 10/00; B33Y 50/00; B29C 64/386

(52) U.S. Cl. CPC C12N 5/0062; C12N 5/0686; C12N 5/0688; B29C 64/124; B29K 2105/0002; B33Y 50/00; B29C 64/386; C12N 2513/00; B33Y 10/00

(57) ABSTRACT

Provided herein are methods and systems for bio-printing of three-dimensional cell-containing matrixes. Further, provided herein are methods and systems for generating a three-dimensional (3D) structure corresponding to a biological material, such as a kidney or lung comprising either nephron or alveolar structures. Also provided herein are bio-printed three-dimensional matrices for use in the generation nephron and/or alveolar structures.

### CROSS-REFERENCE

This application is the continuation of International Application No. PCT/US2019/061035, filed on Nov. 12, 2019, which claims the benefit of U.S. Provisional Patent Application No. 62/760,766, filed on Nov. 13, 2018, the contents of each are incorporated herein by reference in their entirety for all purposes.

### BACKGROUND

Tunable nutrient and gas exchange is important for the development of biological components such as cells and tissues as well as the facilitation of chemical reactions or mixing of fluids at the microscale. Methods of constructing such systems are based on two-dimensional structures, highly uniform stacked structures, or random deposition of tubes and structures.

Biological structures and membrane structures with tunable properties such as size, capillary length, and three-dimensional complexity that allow changes in feature size and control of flow may allow for systems that promote component exchange with greater efficiency than random or simple repeated structures.

Terminal ended micro-structures of varied permeability may allow for removal or isolation of specific components. Examples of such structures are presented in biological tissues as terminal lymphatics but can also be applied in the creation of “smart” filters that are applicable in chemical processes, filtration, or isolation of specific components with high specificity and selectivity.

Controlled but varied surface area to volume ratios may be critical for maximizing diffusion and interactions of diffused glasses, liquids, proteins or other components. Furthermore, controlled but varied structures that can isolate, trap, or act as one-way conduits for materials can increase the efficiency and speed of materials recovery. This may be done with or without cells present, for example in the case of a filter that is built out of non-biological materials. Three dimensional volumes in which it is beneficial to have a controlled or tunable distributed oxygen, nutrients or other components benefit from the development of complex structures that have designed variation in surface to volume ratios as well as three-dimensional positioning such that the desired maximal distribution of the oxygen, nutrients or other components occurs at a specific location.

### SUMMARY

In an aspect, the present disclosure provides a method for generating a three-dimensional (3D) structure corresponding to a biological material comprising a subunit having a surface for performing a biological function, comprising: (a) using at least a number of vessels coupled to the subunit over the surface to generate a computer model of the 3D structure comprising the subunit and the vessels; and (b) using one or more computer processors to print the 3D structure according to the computer model from (a), wherein the 3D structure is implantable in a subject.

In some embodiments, the biological material is a kidney. In some embodiments, the biological material is a lung. In some embodiments, the subunit is a glomerulus. In some embodiments, the subunit is a glomerulus with a Bowman's capsule around the glomerulus. In some embodiments, the subunit is an alveolus. In some embodiments, the biological function comprises an exchange of gasses. In some embodiments, the biological function comprises an exchange of a plurality of metabolically active compounds. In some embodiments, the plurality of metabolically active compounds are selected from the group consisting of nutrients, sugars, salts, amino acids, and metabolic wastes. In some embodiments, the biological function comprises a filtration of plasma. In some embodiments, the vessels comprise one or more blood vessels, or one or more lymphatic vessels, or both. In some embodiments, the one or more blood vessels comprise one or more capillaries.

In some embodiments, the subunit and the vessels, coupling to said subunit over said surface, form a superunit. In some embodiments, the generating a computer model of said 3D structure further comprises using one or more computer processors to combine said superunit with one or more other superunits, wherein said 3D structure corresponds to said biological material.

In some embodiments, the methods further comprise using one or more processors to add a plurality of drainage points to the computer model disclosed herein. In some embodiments, the plurality of drainage points is configured to maintain a net positive fluid pressure within the biological material. In some embodiments, the plurality of drainage points are placed based at least in part by a generative design algorithm. In some embodiments, the plurality of drainage points are placed based at least in part on a density of a plurality of capillaries. In some embodiments, the plurality of drainage points are placed based at least in part on a blood pressure of the 3D structure. In some embodiments, the method further comprises using at least in part a generalized location of the vessels coupling to the subunit, walls of the subunit, or both to identify the surface. In some embodiments, the method further comprises determining a surface area of the subunit having the surface. In some embodiments, the determining comprises using at least in part a plurality of three-dimensional estimations derived from a diameter approximation of the subunit or comparing a volume calculation of the 3D structure to a predetermined range of volumes of the biological material to determine the surface area.

In some embodiments, the vessel is a capillary, further comprising using a total surface area of a plurality of capillaries placed within a space to determine the number of vessels. In some embodiments, the vessel is a capillary, further comprising determining a length of the capillary comprising using an oxygen exchange rate between the capillary's volume of biological fluid and the subunit, wherein the subunit couples to the capillary. In some embodiments, the 3D structure is configured to maintain tissue circulatory homeostasis. In some embodiments, the 3D structure comprises a volume from about 0.125 cubic nanometers to about 1,000 cubic centimeters. In some embodiments the 3D structure is printed by: (a) providing a media chamber comprising a medium comprising (i) a plurality of cells and (ii) one or more polymer precursors; and (b) directing at least one energy beam to the medium in the media chamber along at least one energy beam path that is patterned into a three-dimensional (3D) projection in accordance with the computer model for printing the 3D structure in computer memory, to form at least a portion of the 3D structure comprising (i) at least a subset of the plurality of cells, and (ii) a polymer formed from the one or more polymer precursors. In some embodiments, the plurality of cells is selected from the group consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thing segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes.

In another aspect, the present disclosure provides a method for generating a three-dimensional (3D) structure corresponding to a biological material comprising a subunit having a surface for performing a biological function, comprising: (a) using at least a number of vessels coupled to the subunit over the surface to generate a superunit comprising the subunit and the vessels in computer memory; and (b) using one or more computer processors to combine the superunit generated in (a) with one or more other superunits to generate a computer model of the 3D structure corresponding to the biological material.

In some embodiments, the biological material is a kidney. In some embodiments, the biological material is a lung. In some embodiments, the subunit is a glomerulus. In some embodiments, the subunit is a glomerulus with a Bowman's capsule around the glomerulus. In some embodiments, the subunit is an alveolus. In some embodiments, the biological function comprises an exchange of gasses. In some embodiments, the biological function comprises an exchange of a plurality of metabolically active compounds. In some embodiments, the plurality of metabolically active compounds are selected from the group consisting of nutrients, sugars, salts, amino acids, and metabolic wastes. In some embodiments, the biological function comprises a filtration of plasma. In some embodiments, the vessels comprise one or more blood vessels and one or more lymphatic vessels. In some embodiments, the one or more blood vessels comprise one or more capillaries.

In some embodiments, the method may further comprise using the one or more processors to add a plurality of drainage points to the computer model from (a). In some embodiments, the plurality of drainage points is configured to maintain a net positive fluid pressure within the biological material. In some embodiments, the plurality of drainage points are placed based at least in part by a generative design algorithm. In some embodiments, the plurality of drainage points are placed based at least in part on a density of a plurality of capillaries. In some embodiments, the plurality of drainage points are placed based at least in part on a blood pressure of the 3D structure. In some embodiments, the method further comprises using at least in part a generalized location of the vessels coupling to the subunit, walls of the subunit, or both to identify the surface. In some embodiments the method further comprises determining a surface area of the subunit having the surface. In some embodiments, the vessel is a capillary, further comprising using a total surface area of a plurality of capillaries placed within a space to determine the number of vessels.

In some embodiments, the determining comprises using at least in part a plurality of three-dimensional estimations derived from a diameter approximation of the subunit or comparing a volume calculation of the 3D structure to a predetermined range of volumes of the biological material to determine the surface area. In some embodiments, the vessel is a capillary, further comprising determining a length of the capillary comprising using an oxygen exchange rate between the capillary's volume of biological fluid and the subunit, wherein the subunit couples to the capillary. In some embodiments, the 3D structure is configured to maintain tissue circulatory homeostasis. In some embodiments, the 3D structure comprises a volume from about 0.125 cubic nanometers to about 1,000 cubic centimeters.

In some embodiments the 3D structure is printed by: (a) providing a media chamber comprising a medium comprising (i) a plurality of cells and (ii) one or more polymer precursors; and (b) directing at least one energy beam to the medium in the media chamber along at least one energy beam path that is patterned into a three-dimensional (3D) projection in accordance with the computer model for printing the 3D structure in computer memory, to form at least a portion of the 3D structure comprising (i) at least a subset of the plurality of cells, and (ii) a polymer formed from the one or more polymer precursors. In some embodiments, the plurality of cells is selected from the group consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thing segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes.

In another aspect, the present disclosure provides a system for generating a three-dimensional (3D) structure corresponding to a biological material comprising a subunit having a surface for performing a biological function, comprising one or more computer processors that are individually or collectively programmed to: (a) use at least a number of vessels coupled to the subunit over the surface to generate a computer model of the 3D structure comprising the subunit and the vessels; and (b) transmit the computer model from (a) to a 3D printer for printing the 3D structure, wherein the 3D structure is implantable in a subject.

In another aspect, the present disclosure provides a system for generating a three-dimensional (3D) structure corresponding to a biological material comprising a subunit having a surface for performing a biological function, comprising one or more computer processors that are individually or collectively programmed to: (a) use at least a number of vessels coupled to the subunit over the surface to generate a superunit comprising the subunit and the vessels in computer memory; and (b) combine the superunit generated in (a) with one or more other superunits to generate a computer model of the 3D structure corresponding to the biological material.

In another aspect, the present disclosure provides a method for using a three-dimensional (3D) cell-containing matrix, comprising: (a) providing a media chamber comprising a medium comprising (i) a plurality of cells and (ii) one or more polymer precursors; and (b) directing at least one energy beam to the medium in the media chamber along at least one energy beam path that is patterned into a three-dimensional (3D) projection in accordance with computer instructions for printing the 3D cell-containing medical device in computer memory, to form at least a portion of the 3D cell-containing matrix comprising (i) at least a subset of the plurality of cells, and (ii) a polymer formed from the one or more polymer precursors, wherein the 3D cell-containing matrix is implantable in a subject.

In some embodiments, the 3D cell-containing matrix is an alveolar structure. In some embodiments, the 3D cell-containing matrix is a nephron structure. In some embodiments, the 3D cell-containing matrix is a capillary structure. In some embodiments, the plurality of cells is from the subject. In some embodiments, the plurality of cells is selected from the group consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thing segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes. In some embodiments, the 3D cell-containing matrix forms a suture, stent, staple, clip, strand, patch, graft, sheet, tube, pin, or screws.

In some embodiments, the graft is selected from the list consisting of skin implant, uterine lining, neural tissue implant, bladder wall, intestinal tissue, esophageal lining, stomach lining, hair follicle embedded skin, and retina tissue. In some embodiments, the 3D cell-containing matrix comprises a volume from about 0.125 cubic nanometers to about 1,000 cubic centimeters. In some embodiments, the 3D cell-containing matrix further comprises an agent to promote growth of vasculature or nerves. In some embodiments, the agent is selected from the group consisting of growth factors, cytokines, chemokines, antibiotics, anticoagulants, anti-inflammatory agents, opioid pain-relieving agents, non-opioid pain-relieving agents, immune-suppressing agents, immune-inducing agents, monoclonal antibodies, and stem cell proliferating agents.

In another aspect, the present disclosure provides a method of using a three-dimensional (3D) cell-containing matrix, comprising printing the 3D cell-containing matrix comprising a plurality of cells, wherein the 3D cell-containing matrix is implantable in a subject.

In some embodiments, the 3D cell-containing matrix is an alveolar structure. In some embodiments, the 3D cell-containing matrix is a nephron structure. In some embodiments, the 3D cell-containing matrix is a capillary structure. In some embodiments, the plurality of cells is from the subject. In some embodiments, the plurality of cells is selected from the list consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thing segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes.

In some embodiments, the 3D cell-containing matrix forms a suture, stent, staple, clip, strand, patch, graft, sheet, tube, pin, or a screw. In some embodiments, the graft is selected from the list consisting of skin implant, uterine lining, neural tissue implant, bladder wall, intestinal tissue, esophageal lining, stomach lining, hair follicle embedded skin, and retina tissue. In some embodiments, the 3D cell-containing matrix comprises a volume from about 0.125 cubic nanometers to about 1,000 cubic centimeters. In some embodiments, the cell-containing matrix further comprises an agent to promote growth of vasculature or nerves. In some embodiments, the agent is selected from the group consisting of growth factors, cytokines, chemokines, antibiotics, anticoagulants, anti-inflammatory agents, opioid pain-relieving agents, non-opioid pain-relieving agents, immune-suppressing agents, immune-inducing agents, monoclonal antibodies, and stem cell proliferating agents.

In another aspect, the present disclosure provides a method for using a three-dimensional (3D) cell-containing matrix, comprising: (a) providing a media chamber comprising a first medium, wherein the first medium comprises a first plurality of cells and a first polymeric precursor; (b) directing at least one energy beam to the first medium in the media chamber along at least one energy beam path in accordance with computer instructions for printing the 3D cell-containing matrix in computer memory, to subject at least a portion of the first medium in the media chamber to form a first portion of the 3D cell-containing matrix; (c) providing a second medium in the media chamber, wherein the second medium comprises a second plurality of cells and a second polymeric precursor, wherein the second plurality of cells is of a different type than the first plurality of cells; and (d) directing at least one energy beam to the second medium in the media chamber along at least one energy beam path in accordance with the computer instructions, to subject at least a portion of the second medium in the media chamber to form a second portion of the 3D cell-containing matrix, wherein the 3D cell-containing matrix is implantable in a subject.

In some embodiments, the 3D cell-containing matrix is an alveolar structure. In some embodiments, the 3D cell-containing matrix is a nephron structure. In some embodiments, the 3D cell-containing matrix is a capillary structure. In some embodiments, the first and the second plurality of cells is from the subject. In some embodiments, the first and the second plurality of cells are selected from the group consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thing segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes.

In some embodiments, the 3D cell-containing matrix forms a suture, stent, staple, clip, strand, patch, graft, sheet, tube, pin, or a screw. In some embodiments, the graft is selected from the list consisting of skin implant, uterine lining, neural tissue implant, bladder wall, intestinal tissue, esophageal lining, stomach lining, hair follicle embedded skin, and retina tissue. In some embodiments, the 3D cell-containing matrix comprises a volume from about 0.125 cubic nanometers to about 1,000 cubic centimeters.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an embodiment of a system for rapid multi-photon printing of a desired tissue is illustrated.

FIGS. 2A-2D illustrate example stages of the generation of a desired tissue within the media chamber. FIG. 2A illustrates the media chamber containing media comprising a first cell group. FIG. 2B illustrates the media chamber containing media comprising a second cell group. FIG. 2C illustrates delivery of pulses of the multi-photon laser beam to the media. FIG. 2D illustrates an embodiment wherein the cell-containing scaffolding is printed along the bottom of the media chamber containing media.

FIGS. 3A-3C illustrate various embodiments of a laser system. FIG. 3A illustrates an embodiment of a laser system having a single multi-photon laser source. FIG. 3B illustrates an embodiment of a laser system having multiple laser lines. FIG. 3C illustrates an embodiment of a laser system comprising multiple laser lines, photomultipliers (PMTs), and an objective lens.

FIGS. 4A-4C illustrate various embodiments of the printing system. FIG. 4A illustrates an embodiment of the printing system comprising a beam expander, an optical focusing lens, an additional laser focusing lens, and no axicon or TAG lens. FIG. 4B illustrates an embodiment of the printing system comprising a beam expander, an optical focusing lens, an additional laser focusing lens, and an axicon or TAG lens. FIG. 4C illustrates a Z-step projection printing setup comprising a single SLM or DMD for 2D, x, y sheet or hologram projection for printing around cells and resultant structures printed with given Z-steps.

FIGS. 5A-5B illustrate various embodiments of the multi-photon tissue print head. FIG. 5A illustrates an embodiment of the multi-photon tissue print head comprising a single, upright objective lens. FIG. 5B illustrates an embodiment of the multi-photon tissue print head having inverted optics for imaging structures.

FIGS. 6A-6B illustrate embodiments of a removable and attachable fiber optic cable accessory. FIG. 6A illustrates the fiber optic cable accessory and fiber optic cable. FIG. 6B illustrates the fiber optic cable accessory being used to print the desired complex tissue structure.

FIG. 7 illustrates an embodiment wherein the print-head optics includes at least three objectives, wherein each objective includes a fiber optic cable accessory directed into a single media chamber.

FIG. 8 illustrates an embodiment wherein the print-head optics includes at least six objectives, wherein each objective includes a fiber optic cable accessory directed into a separate media chamber such as a separate well of a multi-well plate.

FIG. 9 illustrates embodiments of print-head optics having an array of objectives acting as print heads.

FIG. 10 illustrates objectives programmed to move over the multi-well plate in X and Y directions to deliver the laser beam projections into each well.

FIG. 11 shows a computer system that is programmed or otherwise configured to implement methods provided herein.

FIG. 12 illustrates the optical components and optical path of an embodiment of the printing system without temporal focusing.

FIG. 13 illustrates the optical components and optical path of an additional embodiment of the printing system with temporal focusing.

FIG. 14 illustrates the optical components and optical path of yet another embodiment of the printing system without temporal focusing.

FIG. 15 illustrates a light detection system.

FIGS. 16A-16D illustrate examples of a nephron and capillary structure. FIG. 16A illustrates a front view of the nephron structure. FIG. 16B illustrates a side view of the nephron and capillary structure. FIG. 16C illustrates an isometric view of the nephron and capillary structure. FIG. 16D illustrates an exploded view of the proximal end of the nephron and capillary structure.

FIGS. 17A-17D illustrate examples of a tube structure. FIG. 17A illustrates a top view of the tube structure. FIG. 17B illustrates a side view and exploded view of the tube structure. FIG. 17C illustrates an isometric view of the tube structure. FIG. 17D illustrates a front view of the tube structure.

FIG. 18 shows a cutaway view and an exploded view of one end of the tube structure.

FIG. 19 illustrates an example fluid system set up comprising the tube structure.

FIGS. 20A-20B illustrate example port configurations of the tube structure. FIG. 20A illustrates the various injection ports in the tube. FIG. 20B illustrates example tube structures comprising two channels and two ports.

FIG. 21 illustrates example configurations of tubule arrays.

FIGS. 22A-22C illustrates an example tubule unit. FIG. 22A illustrates a bottom, isometric view of the tubule unit. FIG. 22B illustrates a top, isometric view of the tubule unit. FIG. 22C illustrates a front view of the tubule unit.

FIGS. 23A-23B illustrate example alveolar structures. FIG. 23A illustrates an example of numerous alveolar structures. FIG. 23B illustrates a cutaway view and an exploded view of numerous alveolar structures conjoined with a shared capillary system.

FIGS. 24A-24D illustrate example alveolar designs. FIG. 24A illustrates a first alveolar design. FIG. 24B illustrates the first alveolar design comprising an outlet port. FIG. 24C illustrates a second alveolar design. FIG. 24D shows the printed alveolar structure comprising the second alveolar design.

FIGS. 25A-25B show example end portions of a printed alveolar structure. FIG. 25A shows a cutaway view of one of the ends of a printed alveolar structure. FIG. 25B shows an image of one of the ends of a printed alveolar structure during a positive pressure flow test.

FIGS. 26A-26D illustrate example basket designs. FIG. 26A illustrates a side view of the basket design. FIG. 26B illustrates a top view of the basket design. FIG. 26C illustrates a bottom view of the basket design. FIG. 26D illustrates an isometric view of the basket design.

FIGS. 27A-27B show printed basket designs. FIG. 27A shows microscopy images of a bottom focal plane of the basket design. FIG. 27B shows microscopy images of a top focal plane of the basket design.

FIG. 28 illustrates an example of a capillary bed.

FIGS. 29A-29D show a blood vessel printed using methods and systems described herein.

FIGS. 30A and 30B show cells growing on vasculature structures.

FIGS. 31A and 31B show a glomerular capillary knot with and without a Bowman's capsule.

FIGS. 32A and 32B show a proximal tube and a glomerulus.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” refers to an amount that is near the stated amount by about 10%, 5%, or 1%, including increments therein. For example, “about” or “approximately” may mean a range including the particular value and ranging from 10% below that particular value and spanning to 10% above that particular value.

The term “biological material,” as used herein, generally refers to any material that may serve a chemical or biological function. Biological material may be biologically functional tissue or functional tissue, which may be a biological structure that is capable of serving, or serving, a biomechanical or biological function. Biologically functional tissue may comprise cells that are within diffusion distance from each other, comprises at least one cell type wherein each cell is within diffusion distance of a capillary or vascular network component, facilitates and/or inhibits the fulfillment of protein function, or any combination thereof. Biologically functional tissue may be at least a portion of tissue or an organ, such as a vital organ. In some examples, the biological material may advance drug development; for example, by screening multiple cells or tissue with different therapeutic agents.

Biological material may include a matrix, such as a polymeric matrix, biogel, hydrogel, or polymeric scaffold, including one or more other types of material, such as cells. Biological material may be an organ or organoid (e.g., a kidney, a lung). Biological material may include lymphoid organs and organoids. Biological material may be derived from human or animal sources of primary cells, cell lines, stem cells, stem cell lines, differentiated stem cells, transdifferentiated stem cells, autologous cells, allogeneic cells, pluripotent stem cells, embryonic stem cells, induced pluripotent stem cells, or any combination thereof. Biological material may be in various shapes, sizes or configurations. In some instances, biological material may be consumable by a subject (e.g., an animal), such as meat or meat-like material. In some instances, biological material is from a subject (e.g., a cell culture from a donor). Biological material may comprise one or more subunits configured to impart functionality to the biological material (e.g., the glomerulus of a kidney, the alveoli of the lungs). The biological material may comprise one or more superunits comprising one or more subunits (e.g., the nephron of a kidney comprising a glomerulus).

The term “three-dimensional printing” (also “3D printing”), as used herein, generally refers to a process or method for generating a 3D part (or object). Such process may be used to form a 3D part (or object), such as a 3D biological material.

The term “energy beam,” as used herein, generally refers to a beam of energy. The energy beam may be a beam of electromagnetic energy or electromagnetic radiation. The energy beam may be a particle beam. An energy beam may be a light beam (e.g., gamma waves, x-ray, ultraviolet, visible light, infrared light, microwaves, or radio waves). The light beam may be a coherent light beam, as may be provided by light amplification by stimulated emission of radiation (“laser”). In some examples, the light beam is generated by a laser diode or a multiple diode laser.

The term “allogenic,” as used herein, generally refers to the plurality of cells are obtained from a genetically non-identical donor. For example, allogenic cells are extracted from a donor and returned back to a different, genetically non-identical recipient.

The term “autologous,” as used herein, generally refers to the plurality of cells are obtained from a genetically identical donor. For example, autologous cells are extracted from a patient and returned back to the same, genetically identical individual (e.g., the donor).

The term “pluripotent stem cells” (PSCs), as used herein, generally refers to cells capable, under appropriate conditions, of producing different cell types that are derivatives of all of the 3 germinal layers (i.e., endoderm, mesoderm, and ectoderm). Included in the definition of pluripotent stem cells are embryonic stem cells of various types including human embryonic stem (hES) cells, human embryonic germ (hEG) cells; non-human embryonic stem cells, such as embryonic stem cells from other primates, such as Rhesus stem cells, marmoset stem cells; murine stem cells; stem cells created by nuclear transfer technology, as well as induced pluripotent stem cells (iPSCs).

The term “embryonic stem cells” (ESCs), as used herein, generally refers to pluripotent stem cells that are derived from a blastocyst before substantial differentiation of the cells into the three germ layers (i.e., endoderm, mesoderm, and ectoderm). ESCs include any commercially available or well established ESC cell line such as H9, H1, H7, or SA002.

The term “induced pluripotent stem cells” or “iPSCs,” as used herein, generally refers to somatic cells that have been reprogrammed into a pluripotent state resembling that of embryonic stem cells. Included in the definition of iPSCs are iPSCs of various types including human iPSCs and non-human iPSCs, such as iPSCs derived from somatic cells that are primate somatic cells or murine somatic cells.

The term “energy source,” as used herein, generally refers to a laser, such as a fiber laser, a short-pulsed laser, or a femto-second pulsed laser; a heat source, such as a thermal plate, a lamp, an oven, a heated water bath, a cell culture incubator, a heat chamber, a furnace, or a drying oven; a light source, such as white light, infrared light, ultraviolet (UV) light, near infrared (NIR) light, visible light, or a light emitting diode (LED); a sound energy source, such as an ultrasound probe, a sonicator, or an ultrasound bath; an electromagnetic radiation source, such as a microwave source; or any combination thereof.

The term “biogel,” as used herein, generally refers to a hydrogel, a biocompatible hydrogel, a polymeric hydrogel, a hydrogel bead, a hydrogel nanoparticle, a hydrogel microdroplet, a solution with a viscosity ranging from at least about 10×10⁻⁴ Pascal-second (Pa·s) to about 100 Pa·s or more when measured at 25 degrees Celsius (° C.), a hydrogel comprising non-hydrogel beads, nanoparticles, microparticles, nanorods, nanoshells, liposomes, nanowires, nanotubes, or a combination thereof a gel in which the liquid component is water; a degradable hydrogel; a non-degradable hydrogel; a resorbable hydrogel; a hydrogel comprising naturally-derived polymers; or any combination thereof.

As used herein, the term “non-biological structure” generally refers to a structure that does not contain living cells.

The term “superunit,” as used herein, generally refers to a unit of a biological material comprising one or more smaller subunit. For example, a superunit of a kidney can be a nephron, which comprises a glomerulus subunit. The term superunit and super-structure may be used interchangeably.

### Three-Dimensional (3D) Printed Structures

The present invention provides for the development of vascularized three dimensional complex, linked tubular micro-structures that are designed to promote the exchange or removal of liquids, gases, and, or nutrients in a tunable, controlled format.

Tissue structures require distribution of oxygen, nutrients and removal of by products produced by cellular metabolism. Structures that allow for distribution of nutrients and removal of wastes are maximally efficient with spacing that allows for uniform and complete diffusion of oxygen. Smaller diameter tubes allow for greater surface to volume ratios which facilitate nutrient exchange and waste removal. Placement and distribution of small diameter tubes allows for control of the rates of oxygen and nutrient exchange such that it can be tuned for specific cell types, to facilitate specific chemical reactions, or to introduce finely engineered and designed areas of hypoxia (low oxygen) or low flow.

The present disclosure provides methods and systems for printing a three-dimensional (3D) biological material. In an aspect, a method for printing the 3D biological material comprises providing a media chamber comprising a medium comprising (i) a plurality of cells and (ii) one or more polymer precursors. Next, at least one energy beam may be directed to the medium in the media chamber along at least one energy beam path that is patterned into a 3D projection in accordance with computer instructions for printing the 3D biological material in computer memory. This may form at least a portion of the 3D biological material comprising (i) at least a subset of the plurality of cells, which at least the subset of the plurality of cells comprises cells of at least two different types, and (ii) a polymer formed from the one or more polymer precursors.

Methods and systems of the present disclosure may be used for constructing tubes and/or designing the organization of tubes that facilitate microcirculation of cells, oxygen, liquids, gasses or heterogeneous materials. Methods and systems of the present disclosure may be used for design and organization of capillaries on the order of living tissue for the purpose of gas or nutrient diffusion. In another aspect, the present disclosure provides methods and systems to print valves and channels on the scale of microns for the purpose of fluid dynamic control. In some examples, the channels may be one-way terminal ended channels of various sizes with engineered permeability for the purpose of fluid removal and fluid dynamic control, similar to terminal lymphatics.

Methods and systems of the present disclosure may be used to print multiple layers of a 3D object, such as a 3D biological material, at the same time. Such 3D object may be formed of a polymeric material, a metal, metal alloy, composite material, or any combination thereof. In some examples, the 3D object is formed of a polymeric material, in some cases including biological material (e.g., one or more cells or cellular components). In some cases, the 3D object may be formed by directing an energy beam (e.g., a laser) as a 3D projection (e.g., hologram) to one or more precursors of the polymeric material, to induce polymerization and/or cross-linking to form at least a portion of the 3D object. This may be used to form multiple layers of the 3D object at the same time.

As an alternative, the 3D object may be formed of a metal or metal alloy, such as, e.g., gold, silver, platinum, tungsten, titanium, or any combination thereof. In such a case, the 3D object may be formed by sintering or melting metal particles, as may be achieved, for example, by directing an energy beam (e.g., a laser beam) at a powder bed comprising particles of a metal or metal alloy. In some cases, the 3D object may be formed by directing such energy beam as a 3D projection (e.g., hologram) into the powder bed to facilitate sintering or melting of particles. This may be used to form multiple layers of the 3D object at the same time. The 3D object may be formed of an organic material such as graphene. The 3D object may be formed of an inorganic material such as silicone. In such cases, the 3D object may be formed by sintering or melting organic and/or inorganic particles, as may be achieved, for example, by directing an energy beam (e.g., a laser beam) at a powder bed comprising particles of an organic and/or inorganic material. In some cases, the 3D object may be formed by directing such energy beam as a 3D projection (e.g., hologram) into the powder bed to facilitate sintering or melting of organic and/or inorganic particles.

The depth of the energy beam penetration may be dictated by the interaction of the beam wavelength and the electron field of a given metal, metal alloy, inorganic material, and/or organic material. The organic material may be graphene. The inorganic material may be silicone. These particles may be functionalized or combined in to allow for greater interaction or less interaction with a given energy beam.

In some examples, the at least one energy beam is at least 1, 2, 3, 4, 5, 6, 7, 8, 9, 10, 20, 30, 40, 50, 100, or more energy beams. The at least one energy beam may be or include coherent light. In some cases, the at least one energy beam is a laser beam.

The at least one energy beam may be directed as an image or image set. The image may be fixed with time or changed with time. The at least one energy beam may be directed as a video.

The computer instructions may correspond to a computer model or representation of the 3D biological material. The computer instructions may be part of the computer model. The computer instructions may comprise a set of images corresponding to the 3D biological material.

The at least one energy beam may be directed as a holographic image or video. This may enable different points in the medium to be exposed to the at least one energy beam at the same time, to, for example, induce formation of a polymer matrix (e.g., by polymerization) at multiple layers at the same time. In some cases, a 3D image or video may be projected into the medium at different focal points using, e.g., a spatial light modulator (SLM).

The computer instructions may include and/or direct adjustment of one or more parameters of the at least one energy beam as a function of time during formation of the 3D biological material, such as, for example, application of power to a source of the at least one energy beam (e.g., laser on/off). Such adjustment may be made in accordance with an image or video (e.g., holographic image or video) corresponding to the 3D biological material. Alternatively, or in addition to, the computer instructions may include and/or direct adjustment of a location of a stage upon which the 3D biological material is formed.

In some cases, during or subsequent to formation of the 3D biological material, at least a portion of the at least the subset of the plurality of cells may be subjected to differentiation to form the cells of the at least two different types. This may be employed, for example, by exposing the cells to an agent or subjecting the cells to a condition that induces differentiation. Alternatively, or in addition to, the cells may be subjected to de-differentiation or induction of cell quiescence.

Another aspect of the present disclosure provides a method for printing a 3D biological material, providing a media chamber comprising a first medium. The first medium may comprise a first plurality of cells and a first polymeric precursor. At least one energy beam may be directed to the first medium in the media chamber along at least one energy beam path in accordance with computer instructions for printing the 3D biological material, to subject at least a portion of the first medium in the media chamber to form a first portion of the 3D biological material. Next, a second medium may be provided in the media chamber. The second medium may comprise a second plurality of cells and a second polymeric precursor. The second plurality of cells may be of a different type than the first plurality of cells. Next, at least one energy beam may be directed to the second medium in the media chamber along at least one energy beam path in accordance with the computer instructions, to subject at least a portion of the second medium in the media chamber to form at least a second portion of the 3D biological material.

In another aspect of the present disclosure, a system for printing a 3D biological material comprises a media chamber configured to contain a medium comprising a plurality of cells comprising cells of at least two different types and one or more polymer precursors; at least one energy source configured to direct at least one energy beam to the media chamber; and one or more computer processors operatively coupled to the at least one energy source, wherein the one or more computer processors are individually or collectively programmed to (i) receive computer instructions for printing the 3D biological material from computer memory; and (ii) direct the at least one energy source to direct the at least one energy beam to the medium in the media chamber along at least one energy beam path in accordance with the computer instructions, to subject at least a portion of the polymer precursors to form at least a portion of the 3D biological material.

In another aspect, a system for printing a 3D biological material, comprising: a media chamber configured to contain a medium comprising a plurality of cells and a plurality of polymer precursors; at least one energy source configured to direct at least one energy beam to the media chamber; and one or more computer processors operatively coupled to the at least one energy source, wherein the one or more computer processors are individually or collectively programmed to (i) receive computer instructions for printing the 3D biological material from computer memory; (ii) direct the at least one energy source to direct the at least one energy beam to the medium in the media chamber along at least one energy beam path in accordance with the computer instructions, to subject at least a portion of the polymer precursors to form at least a portion of the 3D biological material; and (iii) direct the at least one energy source to direct the at least one energy beam to a second medium in the media chamber along at least one energy beam path in accordance with the computer instructions, to subject at least a portion of the second medium in the media chamber to form at least a second portion of the 3D biological material, wherein the second medium comprises a second plurality of cells and a second polymeric precursor, wherein the second plurality of cells is of a different type than the first plurality of cells.

In another aspect of the present disclosure, methods for printing a three-dimensional (3D) object, may comprise directing at least one energy beam into a medium comprising one or more precursors, to generate the 3D object comprising a material formed from the one or more precursors, wherein the at least one energy beam is directed into the medium as a 3D projection corresponding to the 3D object.

In another aspect, methods for printing a three-dimensional (3D) biological material, may comprise directing at least one energy beam to: 1) a first medium comprising a first plurality of cells and a first polymeric precursor, and 2) a second medium comprising a second plurality of cells and a second polymeric precursor, to generate a first portion of the 3D biological material and a second portion of the 3D biological material.

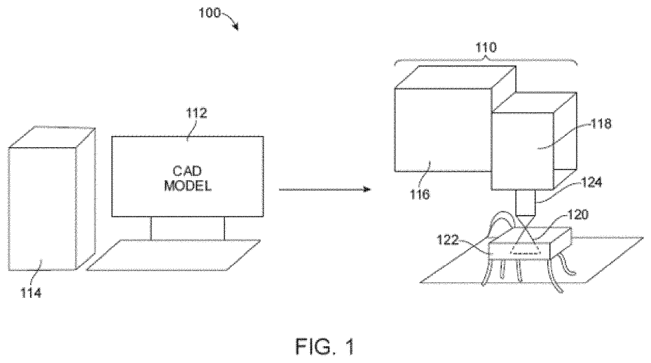

Referring to FIG. 1, an embodiment of a system 100 for rapid multi-photon printing of a desired tissue is illustrated. Here, the system 100 comprises a laser printing system 110 driven by a solid-model computer-aided design (CAD) modeling system 112. In this embodiment, the CAD modeling system 112 comprises a computer 114 which controls the laser printing system 110 based on a CAD model of the desired tissue and additional parameters. The laser printing system 110 comprises a laser system 116 in communication with a multi-photon tissue printing print-head 118 which projects waveforms of a multi-photon laser beam 120 into a media chamber 122 to match the desired structure in complete or in specific parts. The multi-photon tissue print-head 118 includes at least one objective lens 124 that delivers the multi-photon laser beam 120 in the lateral and axial planes of the media chamber 122 to provide a two-dimensional and/or three dimensional and thus holographic projection of the CAD modeled tissue within the media chamber 122. The objective lens 124 may be a water-immersion objective lens, an air objective lens, or an oil-immersion objective lens. Two dimensional and three dimensional holographic projections may be generated simultaneously and projected into different regions by lens control. The media chamber 122 contains media comprised of cells, polymerizable material, and culture medium. The polymerizable material may comprise polymerizable monomeric units that are biologically compatible, dissolvable, and, in some cases, biologically inert. The monomeric units (or subunits) may polymerize, cross-link, or react in response to the multi-photon laser beam 120 to create cell containing structures, such as cell matrices and basement membrane structures, specific to the tissue to be generated. The monomeric units may polymerize and/or cross-link to form a matrix. In some cases, the polymerizable monomeric units may comprise mixtures of collagen with other extracellular matrix components including but not limited to elastin and hyaluronic acid to varying percentages depending on the desired tissue matrix.

Non-limiting examples of extracellular matrix components used to create cell containing structures may include proteoglycans such as heparan sulfate, chondroitin sulfate, and keratan sulfate, non-proteoglycan polysaccharide such as hyaluronic acid, collagen, and elastin, fibronectin, laminin, nidogen, or any combination thereof. These extracellular matrix components may be functionalized with acrylate, diacrylate, methacrylate, cinnamoyl, coumarin, thymine, or other side-group or chemically reactive moiety to facilitate cross-linking induced directly by multi-photon excitation or by multi-photon excitation of one or more chemical doping agents. In some cases, photopolymerizable macromers and/or photopolymerizable monomers may be used in conjunction with the extracellular matrix components to create cell-containing structures. Non-limiting examples of photopolymerizable macromers may include polyethylene glycol (PEG) acrylate derivatives, PEG methacrylate derivatives, and polyvinyl alcohol (PVA) derivatives. In some instances, collagen used to create cell containing structure may be fibrillar collagen such as type I, II, III, V, and XI collagen, facit collagen such as type IX, XII, and XIV collagen, short chain collagen such as type VIII and X collagen, basement membrane collagen such as type IV collagen, type VI collagen, type VII collagen, type XIII collagen, or any combination thereof.

Specific mixtures of monomeric units may be created to alter the final properties of the polymerized biogel. This base print mixture may contain other polymerizable monomers that are synthesized and not native to mammalian tissues, comprising a hybrid of biologic and synthetic materials. An example mixture may comprise about 0.4% w/v collagen methacrylate plus the addition of about 50% w/v polyethylene glycol diacrylate (PEGDA). Photoinitiators to induce polymerization may be reactive in the ultraviolet (UV), infrared (IR), or visible light range. Examples of two such photo initiators are Eosin Y (EY) and triethanolamine (TEA), that when combined may polymerize in response to exposure to visible light (e.g., wavelengths of about 390 to 700 nanometers). Non-limiting examples of photoinitiators may include azobisisobutyronitrile (AIBN), benzoin derivatives, benziketals, hydroxyalkylphenones, acetophenone derivatives, trimethylolpropane triacrylate (TPT), acryloyl chloride, benzoyl peroxide, camphorquinone, benzophenone, thioxanthones, and 2-hydroxy-1-[4-(hydroxyethoxy)phenyl]-2-methyl-1-propanone. Hydroxyalkylphenones may include 4-(2-hydroxyethylethoxy)-phenyl-(2-hydroxy-2-methyl propyl) ketone (Irgacure® 295), 1-hidroxycyclohexyl-1-phenyl ketone (Irgacure® 184) and 2,2-dimethoxy-2-phenylacetophenone (Irgacure® 651). Acetophenone derivatives may include 2,2-dimethoxy-2-phenylacetophenone (DMPA). Thioxanthones may include isopropyl thioxanthone.

Specific mixtures of monomeric units of biological materials may be created to alter the final properties of the polymerized biogel, an example mixture may include about 1 mg/mL type I collagen-methacrylate, about 0.5 mg/mL type III collagen, about 0.2 mg/mL methacrylated hyaluronic acid, about 0.1% Eosin Y, and about 0.1% triethanolamine.

In some cases, the polymerized biogel may comprise at least about 0.01% of a photoinitiator. In some cases, the polymerized biogel may comprise about 10% of a photoinitiator or more. In some cases, the polymerized biogel comprises about 0.1% of a photoinitiator. In some cases, the polymerized biogel may comprise about 0.01% to about 0.05%, about 0.01% to about 0.1%, about 0.01% to about 0.2%, about 0.01% to about 0.3%, about 0.01% to about 0.4%, about 0.01% to about 0.5%, about 0.01% to about 0.6%, about 0.7% to about 0.8%, about 0.9% to about 1%, about 0.01% to about 2%, about 0.01% to about 3%, about 0.01%% to about 4%, about 0.01% to about 5%, about 0.01% to about 6%, about 0.01% to about 7%, about 0.01% to about 8%, about 0.01% to about 9%, or about 0.01% to about 10% of a photoinitiator.

The polymerized biogel may comprise about 0.05% of a photoinitiator. The polymerized biogel may comprise 0.1% of a photoinitiator. The polymerized biogel may comprise about 0.2% of a photoinitiator. The polymerized biogel may comprise about 0.3% of a photoinitiator. The polymerized biogel may comprise about 0.4% of a photoinitiator. The polymerized biogel may comprise about 0.5% of a photoinitiator. The polymerized biogel may comprise about 0.6% of a photoinitiator. The polymerized biogel may comprise about 0.7% of a photoinitiator. The polymerized biogel may comprise about 0.8% of a photoinitiator. The polymerized biogel may comprise about 0.9% of a photoinitiator. The polymerized biogel may comprise about 1% of a photoinitiator. The polymerized biogel may comprise about 1.1% of a photoinitiator. The polymerized biogel may comprise about 1.2% of a photoinitiator. The polymerized biogel may comprise about 1.3% of a photoinitiator. The polymerized biogel may comprise about 1.4% of a photoinitiator. The polymerized biogel may comprise about 1.5% of a photoinitiator. The polymerized biogel may comprise about 1.6% of a photoinitiator. The polymerized biogel may comprise about 1.7% of a photoinitiator. The polymerized biogel may comprise about 1.8% of a photoinitiator. The polymerized biogel may comprise about 1.9% of a photoinitiator. The polymerized biogel may comprise about 2% of a photoinitiator. The polymerized biogel may comprise about 2.5% of a photoinitiator. The polymerized biogel may comprise about 3% of a photoinitiator. The polymerized biogel may comprise about 3.5% of a photoinitiator. The polymerized biogel may comprise about 4% of a photoinitiator. The polymerized biogel may comprise about 4.5% of a photoinitiator. The polymerized biogel may comprise about 5% of a photoinitiator. The polymerized biogel may comprise about 5.5% of a photoinitiator. The polymerized biogel may comprise about 6% of a photoinitiator. The polymerized biogel may comprise about 6.5% of a photoinitiator. The polymerized biogel may comprise about 7% of a photoinitiator. The polymerized biogel may comprise about 7.5% of a photoinitiator. The polymerized biogel may comprise about 8% of a photoinitiator. The polymerized biogel may comprise about 8.5% of a photoinitiator. The polymerized biogel may comprise about 9% of a photoinitiator. The polymerized biogel may comprise about 9.5% of a photoinitiator. The polymerized biogel may comprise about 10% of a photoinitiator.

In some cases, the polymerized biogel may comprise at least about 10% of a photopolymerizable macromer and/or photopolymerizable monomer. In some cases, the polymerized biogel may comprise about 99% or more of a photopolymerizable macromer and/or photopolymerizable monomer. In some cases, the polymerized biogel may comprise about 50% of a photopolymerizable macromer and/or photopolymerizable monomer. In some cases, the polymerized biogel may comprise about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, or about 10% to about 99% of a photopolymerizable macromer and/or photopolymerizable monomer.

The polymerized biogel may comprise about 10% of a photopolymerizable macromer and/or photopolymerizable monomer. The polymerized biogel may comprise about 15% of a photopolymerizable macromer and/or photopolymerizable monomer. The polymerized biogel may comprise about 20% of a photopolymerizable macromer and/or photopolymerizable monomer. The polymerized biogel may comprise about 25% of a photopolymerizable macromer and/or photopolymerizable monomer. The polymerized biogel may comprise about 30% of a photopolymerizable macromer and/or photopolymerizable monomer. The polymerized biogel may comprise about 35% of a photopolymerizable macromer and/or photopolymerizable monomer. The polymerized biogel may comprise about 40% photopolymerizable macromer and/or photopolymerizable monomer. The polymerized biogel may comprise about 45% of a photopolymerizable macromer and/or photopolymerizable monomer. The polymerized biogel may comprise about 50% of a photopolymerizable macromer and/or photopolymerizable monomer. The polymerized biogel may comprise about 55% of a photopolymerizable macromer and/or photopolymerizable monomer. The polymerized biogel may comprise about 60% of a photopolymerizable macromer and/or photopolymerizable monomer. The polymerized biogel may comprise about 65% of a photopolymerizable macromer and/or photopolymerizable monomer. The polymerized biogel may comprise about 70% of a photopolymerizable macromer and/or photopolymerizable monomer. The polymerized biogel may comprise about 75% of a photopolymerizable macromer and/or photopolymerizable monomer. The polymerized biogel may comprise about 80% of a photopolymerizable macromer and/or photopolymerizable monomer. The polymerized biogel may comprise about 85% of a photopolymerizable macromer and/or photopolymerizable monomer. The polymerized biogel may comprise about 90% of a photopolymerizable macromer and/or photopolymerizable monomer. The polymerized biogel may comprise about 95% of a photopolymerizable macromer and/or photopolymerizable monomer. The polymerized biogel may comprise about 96% of a photopolymerizable macromer and/or photopolymerizable monomer. The polymerized biogel may comprise about 97% of a photopolymerizable macromer and/or photopolymerizable monomer. The polymerized biogel may comprise about 98% of a photopolymerizable macromer and/or photopolymerizable monomer. The polymerized biogel may comprise about 99% of a photopolymerizable macromer and/or photopolymerizable monomer.

Two-photon absorption is non-linear and cannot be accurately predicted or calculated based on single photon absorption properties of a chemical. A photo-reactive chemical may have a peak, two-photon absorption at or around double the single photon absorption or be slightly-redshifted in absorption spectra. Therefore, wavelengths at or about 900 nanometers through about 1400 nanometers may be used for polymerization of monomeric materials by exciting mixtures of catalysts of the polymerization reaction, for example EY or TEA. Single wavelength polymerization may be sufficient for creating all structural elements, however to further speed up the printing process, multiple wavelengths may be employed simultaneously through the same printing apparatus and into the same printing chamber.

Premixing or pre-reacting of polymerizable monomeric units with catalysts comprising differing absorption bands may allow for printing at different wavelengths to form different substrate-based structural elements simultaneously within the media chamber 122. Thus, certain structural elements may be generated by tuning the excitation wavelength of the laser to a particular wavelength, and then other structural elements may be generated around the existing elements by tuning another or the same laser to a different excitation wavelength that may interact with a distinct photoinitiator that initiates polymerization of one material base with greater efficiency. Likewise, different wavelengths may be used for different structural elements, wherein increased rigidity is desired in some locations and soft or elastic structures are desired in other locations. Because of the different physical properties of polymerizable materials this may allow for potentially more rigid, soft, or elastic structures to be created in the same print step with the same cells by simply tuning the excitation wavelength of the laser electronically, by switching between different lasers, or by simultaneously projecting two different wavelengths.

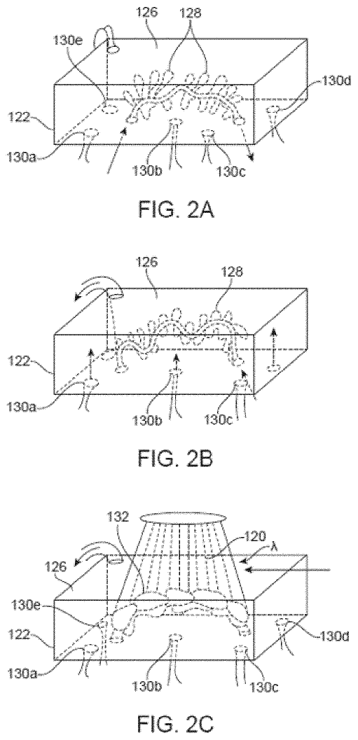

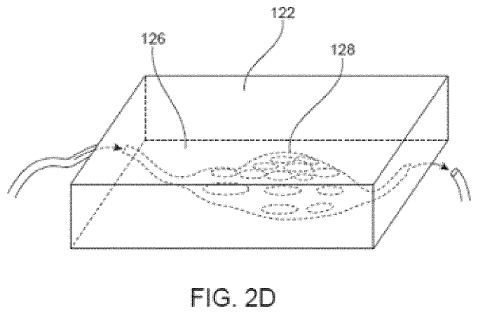

FIGS. 2A-2C illustrate example stages of the generation of a desired tissue within the media chamber 122. FIG. 2A illustrates the media chamber 122 containing media 126 comprised of a first cell group, polymerizable material and culture medium. In this embodiment, pulses of the multi-photon laser beam 120 may be delivered to the media 126 according to the CAD model corresponding to the vascular structure and microvasculature of the desired tissue. In some instances, the first cell group may comprise vascular and/or microvascular cells including but not limited to endothelial cells, microvascular endothelial cells, pericytes, smooth muscle cells, fibroblasts, endothelial progenitor cells, stem cells, or any combination thereof Thus, portions of the media 126 may polymerize, cross-link or react to form cell-containing scaffolding 128 representing the vasculature and microvasculature of the desired tissue. In this embodiment, the media 126 may then be drained through a first port 130a, a second port 130b, a third port 130c, a fourth port 130d, and a fifth port 130e to remove the first cell group and associated media. In some instances, the media chamber 122 may comprise at least one port. In some instances, the media chamber 122 may comprise a plurality of ports ranging from at least one port to 100 ports at most. The media chamber 122 may comprise at least two ports. The media chamber 122 may comprise at least three ports. The media chamber 122 may comprise at least four ports. The media chamber 122 may comprise at least five ports.

Referring to FIG. 2B, the media chamber 122 may be filled with media 126 containing a second cell group, polymerizable material and culture medium through ports 130. This second cell group may be used to generate tissue structures around the existing cell-containing scaffolding 128. In some instances, the cell-containing scaffolding 128 may be a vascular scaffold. The printed vascular scaffolding may comprise endothelial cells, vascular endothelial cells, pericytes, smooth muscle cells, fibroblasts, endothelial progenitor cells, stem cells, or any combination thereof.

The first cell group and/or second cell group may comprise endothelial cells, microvascular endothelial cells, pericytes, smooth muscle cells, fibroblasts, endothelial progenitor cells, lymph cells, T cells such as helper T cells and cytotoxic T cells, B cells, natural killer (NK) cells, reticular cells, hepatocytes, or any combination thereof. The first cell group and/or second cell group may comprise exocrine secretory epithelial cells, hormone-secreting cells, epithelial cells, nerve cells, adipocytes, kidney cells, pancreatic cells, pulmonary cells, extracellular matrix cells, muscle cells, blood cells, immune cells, germ cells, interstitial cells, or any combination thereof.

The first cell group and/or second cell group may comprise exocrine secretory epithelial cells including but not limited to salivary gland mucous cells, mammary gland cells, sweat gland cells such as eccrine sweat gland cell and apocrine sweat gland cell, sebaceous gland cells, type II pneumocytes, or any combination thereof.

The first cell group and/or second cell group may comprise hormone-secreting cells including but not limited to anterior pituitary cells, intermediate pituitary cells, magnocellular neurosecretory cells, gut tract cells, respiratory tract cells, thyroid gland cells, parathyroid gland cells, adrenal gland cells, Leydig cells, theca interna cells, corpus luteum cells, juxtaglomerular cells, macula densa cells, peripolar cells, mesangial cells, pancreatic islet cells such as alpha cells, beta cells, delta cells, PP cells, and epsilon cells, or any combination thereof.

The first cell group and/or second cell group may comprise epithelial cells including but not limited to keratinizing epithelial cells such as keratinocytes, basal cells, and hair shaft cells, stratified barrier epithelial cells such as surface epithelial cells of stratified squamous epithelium, basal cells of epithelia, and urinary epithelium cells, or any combination thereof.

The first cell group and/or second cell group may comprise nerve cells or neurons including but not limited to sensory transducer cells, autonomic neuron cells, peripheral neuron supporting cells, central nervous system neurons such as interneurons, spindle neurons, pyramidal cells, stellate cells, astrocytes, oligodendrocytes, ependymal cells, glial cells, or any combination thereof.

The first cell group and/or second cell group may comprise kidney cells including but not limited to, parietal cells, podocytes, mesangial cells, distal tubule cells, proximal tubule cells, Loop of Henle thin segment cells, collecting duct cells, interstitial kidney cells, or any combination thereof.

The first cell group and/or second cell group may comprise pulmonary cells including, but not limited to type I pneumocyte, alveolar cells, capillary endothelial cells, alveolar macrophages, bronchial epithelial cells, bronchial smooth muscle cells, tracheal epithelial cells, small airway epithelial cells, or any combination thereof.

The first cell group and/or second cell group may comprise extracellular matrix cells including, but not limited to epithelial cells, fibroblasts, pericytes, chondrocytes, osteoblasts, osteocytes, osteoprogenitor cells, stellate cells, hepatic stellate cells, or any combination thereof.

The first cell group and/or second cell group may comprise muscle cells including, but not limited to skeletal muscle cells, cardiomyocytes, Purkinje fiber cells, smooth muscle cells, myoepithelial cells, or any combination thereof.

The first cell group and/or second cell group may comprise blood cells and/or immune cells including, but not limited to erythrocytes, megakaryocytes, monocytes, macrophages, osteoclasts, dendritic cells, microglial cells, neutrophils, eosinophils, basophils, mast cells, helper T cells, suppressor T cells, cytotoxic T cells, natural killer T cells, B cells, natural killer (NK) cells, reticulocytes, or any combination thereof.

FIG. 2C illustrates delivery of pulses of the multi-photon laser beam 120 to the media 126 according to the CAD model of the remaining tissue. Thus, additional portions of the media 126 may polymerize, cross-link or react to form cell-containing structures 132 around the existing cell-containing scaffolding 128 (no longer visible) without damaging or impacting the existing vascular scaffolding 128. The steps of draining the media 126, refilling with new media 126 and delivering laser energy may be repeated any number of times to create the desired complex tissue.

FIG. 2D illustrates an embodiment wherein the cell-containing scaffolding 128 may be printed along the bottom of the media chamber 122 containing media 126. Thus, the scaffolding 128 may not be free standing or free floating. The multi-channel input may reduce shear forces associated with bulk flow from one direction, uneven washing of fine structures as bulk flow may not wash unwanted cells from small features, and uneven distribution of new cell containing media as it is cycled into the tissue printing chamber. The multiple inputs may come from the top, bottom, sides or all three simultaneously. Multiple inputs are particularly desired for tissue printing because cell-containing structures are relatively fragile and potentially disrupted by the application of fluid forces associated with media exchange through the chamber. FIG. 2D shows that the tissues may be printed above the bottom plate of the media chamber. In some embodiments, the cells and tissue may be printed flush against the bottom of the media chamber. Additionally, this design may allow for easy transport of printed tissues and positioning under a laser print head (focusing objective) and is a closed system that may allow for media exchange and printing to occur without exposure to room air. This may be desired as exposure to room air may introduce infectious agents into the cell culture media which may disrupt or completely destroy the development of useful tissues.

### Laser Printing Systems

In an aspect, the present disclosure provides systems for printing a three-dimensional (3D) biological material. The x, y, and z dimensions may be simultaneously accessed by the systems provided herein. A system for printing a 3D biological material may comprise a media chamber configured to contain a medium comprising a plurality of cells comprising cells and one or more polymer precursors. The plurality of cells may comprise cells of at least one type. The plurality of cells may comprise cells of at least two different types. The system may comprise at least one energy source configured to direct at least one energy beam to the media chamber. The system may comprise at least one energy source configured to direct at least one energy beam to the media chamber and/or to the cell-containing chamber. The system may comprise one or more computer processors operatively coupled to the at least one energy source, wherein the one or more computer processors may be individually or collectively programmed to: receive computer instructions for printing the 3D biological material from computer memory; and direct the at least one energy source to direct the at least one energy beam to the medium in the media chamber along at least one energy beam path in accordance with the computer instructions, to subject at least a portion of the polymer precursors to form at least a portion of the 3D biological material.

In another aspect, the present disclosure provides an additional system for printing a 3D biological material, comprising a media chamber configured to contain a medium comprising a plurality of cells and a plurality of polymer precursors. The system may comprise at least one energy source configured to direct at least one energy beam to the media chamber. In addition, the system may comprise one or more computer processors that may be operatively coupled to the at least one energy source. The one or more computer processors may be individually or collectively programmed to: (i) receive computer instructions for printing the 3D biological material from computer memory; (ii) direct the at least one energy source to direct the at least one energy beam to the medium in the media chamber along at least one energy beam path in accordance with the computer instructions, to subject at least a portion of the polymer precursors to form at least a portion of the 3D biological material; and (iii) direct the at least one energy source to direct the at least one energy beam to a second medium in the media chamber along at least one energy beam path in accordance with the computer instructions, to subject at least a portion of the second medium in the media chamber to form at least a second portion of the 3D biological material, wherein the second medium comprises a second plurality of cells and a second polymeric precursor, wherein the second plurality of cells is of a different type than the first plurality of cells.

The one or more computer processors are individually or collectively programmed to generate a point-cloud representation or lines-based representation of the 3D biological material in computer memory, and use the point-cloud representation or lines-based representation to generate the computer instructions for printing the 3D biological material in computer memory. The one or more computer processors may be individually or collectively programmed to direct the at least one energy source to direct the at least one energy beam along one or more additional energy beam paths to form at least another portion of the 3D biological material.

The system may comprise one or more computer processors operatively coupled to at least one energy source and/or to at least one light patterning element. The point-cloud representation or the lines-based representation of the computer model may be a holographic point-cloud representation or a holographic lines-based representation. The one or more computer processors may be individually or collectively programmed to use the light patterning element to re-project the holographic image as illuminated by the at least one energy source.

In some cases, one or more computer processors may be individually or collectively programmed to convert the point-cloud representation or lines-based representation into an image. The one or more computer processors may be individually or collectively programmed to project the image in a holographic manner. The one or more computer processors may be individually or collectively programmed to project the image as a hologram. The one or more computer processors may be individually or collectively programmed to project the image as partial hologram. In some cases, one or more computer processors may be individually or collectively programmed to convert the point-cloud representation or lines-based representation of a complete image set into a series of holographic images via an algorithmic transformation. This transformed image set may then be projected in sequence by a light patterning element, such as a spatial light modulator (SLM) or digital mirror device (DMD), through the system, recreating the projected image within the printing chamber with the projected light that is distributed in 2D and or 3D simultaneously. An expanded or widened laser beam may be projected onto the SLMs and/or DMDs, which serve as projection systems for the holographic image. In some cases, one or more computer processors may be individually or collectively programmed to project the image in a holographic manner. In some cases, one or more computer processors may be individually or collectively programmed to project the images all at once or played in series as a video to form a larger 3D structure in a holographic manner.

Holography is a technique that projects a multi-dimensional (e.g., 2D and/or 3D) holographic image or a hologram. When a laser that can photo-polymerize a medium is projected as a hologram, the laser may photopolymerize, solidify, cross-link, bond, harden, and/or change a physical property of the medium along the projected laser light path; thus, the laser may allow for the printing of 3D structures. Holography may require a light source, such as a laser light or coherent light source, to create the holographic image. The holographic image may be constant over time or varied with time (e.g., a holographic video). Furthermore, holography may require a shutter to open or move the laser light path, a beam splitter to split the laser light into separate paths, mirrors to direct the laser light paths, a diverging lens to expand the beam, and additional patterning or light directing elements.

A holographic image of an object may be created by expanding the laser beam with a diverging lens and directing the expanded laser beam onto the hologram and/or onto at least one pattern forming element, such as, for example a spatial light modulator or SLM. The pattern forming element may encode a pattern comprising the holographic image into a laser beam path. The pattern forming element may encode a pattern comprising a partial hologram into a laser beam path. Next, the pattern may be directed towards and focused in the medium chamber containing the printing materials (i.e., the medium comprising the plurality of cells and polymeric precursors), where it may excite a light-reactive photoinitiator found in the printing materials (i.e., in the medium). Next, the excitation of the light-reactive photoinitiator may lead to the photopolymerization of the polymeric-based printing materials and forms a structure in the desired pattern (i.e., holographic image). In some cases, one or more computer processors may be individually or collectively programmed to project the holographic image by directing an energy source along distinct energy beam paths.

In some cases, at least one energy source may be a plurality of energy sources. The plurality of energy sources may direct a plurality of the at least one energy beam. The energy source may be a laser. In some examples, the laser may be a fiber laser. For example, a fiber laser may be a laser with an active gain medium that includes an optical fiber doped with rare-earth elements, such as, for example, erbium, ytterbium, neodymium, dysprosium, praseodymium, thulium and/or holmium. The energy source may be a short-pulsed laser. The energy source may be a femto-second pulsed laser. The femtosecond pulsed laser may have a pulse width less than or equal to about 500 femtoseconds (fs), 250, 240, 230, 220, 210, 200, 150, 100, 50 fs, 40 fs, 30 fs, 20 fs, 10 fs, 9 fs, 8 fs, 7 fs, 6 fs, 5 fs, 4 fs, 3 fs, 2 fs, 1 fs, or less. The femtosecond pulsed laser may be, for example, a titanium:sapphire (Ti:Sa) laser. The at least one energy source may be derived from a coherent light source.

The coherent light source may provide light with a wavelength from about 300 nanometers (nm) to about 5 millimeters (mm). The coherent light source may comprise a wavelength from about 350 nm to about 1800 nm, or about 1800 nm to about 5 mm. The coherent light source may provide light with a wavelength of at least about 300 nm, 400 nm, 500 nm, 600 nm, 700 nm, 800 nm, 900 nm, 1 mm, 1.1 mm, 1.2, mm, 1.3 mm, 1.4 mm, 1.5 mm, 1.6 mm, 1.7 mm, 1.8 mm, 1.9 mm, 2 mm, 3 mm, 4 mm, 5 mm, or greater.

The computer processors may be individually or collectively programmed to direct the at least one energy source to direct the at least one energy beam along one or more additional energy beam paths to form at least another portion of the 3D biological material. The one or more additional energy beam paths may be along an x axis, an x and y plane, or the x, y, and z planes. The one or more additional energy beam paths may be along an x axis. The one or more additional energy beam paths may be along a y axis. The one or more additional energy beam paths may be along a z axis. The energy beam path may converge with one or more other beams on the same axis. The one or more additional energy beam paths may be in the x and y plane. The one or more additional energy beam paths may be in the x and z plane. The one or more additional energy beam paths may be in the y and z plane. The one or more additional energy beam paths may be in the x, y, and z planes.

The system may further comprise at least one objective lens for directing the at least one energy beam to the medium in the media chamber. In some instances, at least one objective lens may comprise a water-immersion objective lens. In some instances, at least one objective lens may comprise a water-immersion objective lens. In some instances, at least one objective lens may comprise a water dipping objective lens. In some instances, at least one objective lens may comprise an oil immersion objective lens. In some instances, at least one objective lens may comprise an achromatic objective lens, a semi-apochromatic objective lens, a plans objective lens, an immersion objective lens, a Huygens objective lens, a Ramsden objective lens, a periplan objective lens, a compensation objective lens, a wide-field objective lens, a super-field objective lens, a condenser objective lens, or any combination thereof. Non-limiting examples of a condenser objective lens may include an Abbe condenser, an achromatic condenser, and a universal condenser.

The one or more computer processors may be individually or collectively programmed to receive images of the edges of the 3D biological material. The one or more computer processors may be individually or collectively programmed to receive images of the exterior surfaces of the 3D biological material. The one or more computer processors may be individually or collectively programmed to receive images of the interior surfaces of the 3D biological material. The one or more computer processors may be individually or collectively programmed to receive images of the interior of the 3D biological material.

The one or more computer processors may be individually or collectively programmed to direct linking of the 3D biological material with other tissue, which linking may be in accordance with the computer instructions. The one or more computer processors may be individually or collectively programmed to directly link, merge, bond, or weld 3D printed material with already printed structures, where linking is in accordance with the computer model. In some cases, linking of the 3D biological material with other tissue may involve chemical cross-linking, mechanical linking, and/or cohesively coupling.

In another aspect, the system may comprise a media chamber configured to contain a medium comprising a plurality of cells and a plurality of polymer precursors. The system may comprise at least one energy source configured to direct at least one energy beam to the media chamber. The system may comprise one or more computer processors operatively coupled to at least one energy source, wherein the one or more computer processors are individually or collectively programmed to: receive a computer model of the 3D biological material in computer memory; generate a point-cloud representation or lines-based representation of the computer model of the 3D biological material in computer memory; direct the at least one energy source to direct the at least one energy beam to the medium in the media chamber along at least one energy beam path in accordance with the computer model of the 3D biological material, to subject at least a portion of the polymer precursors to form at least a portion of the 3D biological material; and direct the at least one energy source to direct the at least one energy beam to a second medium in the media chamber along at least one energy beam path in accordance with the computer model of the 3D biological material, to subject at least a portion of the second medium in the media chamber to form at least a second portion of the 3D biological material, wherein the second medium comprises a second plurality of cells and a second polymeric precursor, wherein the second plurality of cells is of a different type than the first plurality of cells.

In laser printing of cellular structures, rapid three-dimensional structure generation using minimally toxic laser excitation is critical for maintaining cell viability and in the case of functional tissue printing, necessary for large-format, high resolution, multicellular tissue generation. Other methods of two-photon printing may rely upon raster-scanning of two-photon excitation in a two-dimensional plane (x, y) (e.g., selective laser sintering), while moving the microscope or stage in the z direction to create a three-dimensional structure. This technique may be prohibitively slow for large format multicellular tissue printing such that cell viability may be unlikely to be maintained during printing of complex structures. Certain hydrogels with high rates of polymerization may also be utilized for two-dimensional projection of tissue sheets that are timed such that one slice of a structure is projected with each step in in an x, y, or z plane. Additionally, mixed plane angles representing a sheet or comprising an orthogonal slice may also be utilized. In the case of rapidly polymerizing hydrogels, these projections may work in time-scales that are compatible with tissue printing whereas laser sintering or raster scanning (e.g., layer-by-layer deposition) may be prohibitively slow for building a complex structure.

The laser printing system 110 of the present disclosure may be equipped with an objective lens 124 that may allow for focusing of the three-dimensional or two-dimensional holographic projection in the lateral and axial planes for rapid creation of cell containing structures. The objective lens 124 may be a water-immersion objective lens, an air objective lens, or an oil-immersion objective lens. In some cases, the laser printing system 110 may include a laser system 116 having multiple laser lines and may be capable of three-dimensional holographic projection of images for photolithography via holographic projection into cell containing media.

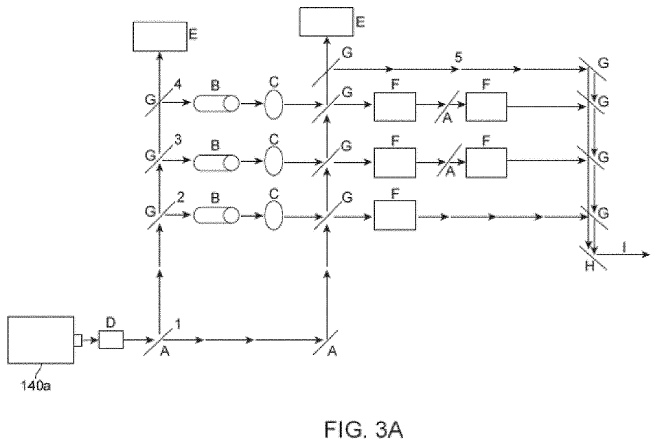

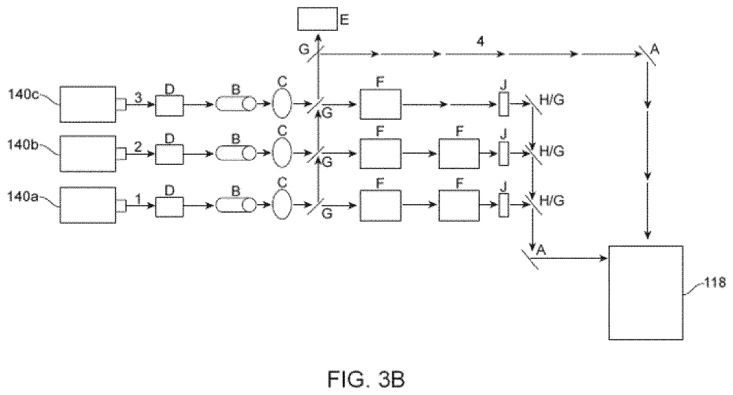

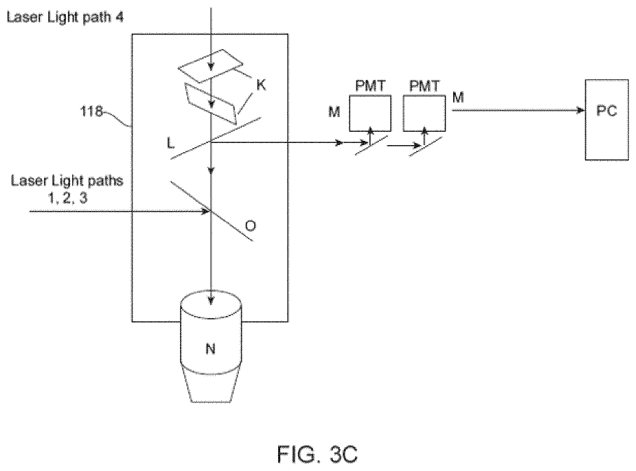

FIG. 3A illustrates an embodiment of a laser system 116 having a first multi-photon laser source 140a. Here, the laser line one, multi-photon laser beam may be reflected by a spatial light modulator (SLM) with a video rate or faster re-fresh rate for image projection, to allow for rapid changes in the three-dimensional structure being projected.

In some cases, spatial light modulators (SLMs) may be used to print a 3D biological material. In some cases, the method presented herein may comprise receiving a computer model of the 3D biological material in computer memory and further processing the computer model such that the computer model is “sliced” into layers, creating a two-dimensional (2D) image of each layer. The computer model may be a computer-aided design (CAD) model. The system disclosed herein may comprise at least one computer processor which may be individually or collectively programmed to calculate a laser scan path based on the “sliced” computer model, which determines the boundary contours and/or fill sequences of the 3D biological material to be printed. Holographic 3D printing may be used with one or more polymer precursors described herein. SLM may be used with two or more polymer precursors described herein.

A spatial light modulator (SLM) is an electrically programmable device that can modulate amplitude, phase, polarization, propagation direction, intensity or any combination thereof of light waves in space and time according to a fixed spatial (i.e., pixel) pattern. The SLM may be based on translucent, e.g., liquid crystal display (LCD) microdisplays. The SLM may be based on reflective, e.g., liquid crystal on silicon (LCOS) microdisplays. The SLM may be a microchannel spatial light modulator (MSLM), a parallel-aligned nematic liquid crystal spatial light modulator (PAL-SLM), a programmable phase modulator (PPM), a phase spatial light modulator (LCOS-SLM), or any combination thereof. An LCOS-SLM may comprise a chip that includes a liquid crystal layer arranged on top of a silicon substrate. A circuit may be built on the chip's silicon substrate by using semiconductor technology. A top layer of the LCOS-SLM chip may contain aluminum electrodes that are able to control their voltage potential independently. A glass substrate may be placed on the silicon substrate while keeping a constant gap, which is filled by the liquid crystal material. The liquid crystal molecules may be aligned in parallel by the alignment control technology provided in the silicon and glass substrates. The electric field across this liquid crystal layer can be controlled pixel by pixel. The phase of light can be modulated by controlling the electric field; a change in the electric field may cause the liquid crystal molecules to tilt accordingly. When the liquid crystal molecules tilt, the liquid crystal refractive indexes may change further changing the optical path length and thus, causing a phase difference.

An SLM may be used to print the 3D biological material. A liquid crystal on silicon (LCOS)-SLM may be used to print the 3D biological material. A liquid crystal SLM may be used to print the 3D biological material. The SLM may be used to project a point-cloud representation or a lines-based representation of a computer model of the 3D biological material. The methods disclosed herein may comprise converting the point-cloud representation or lines-based representation into a holographic image. The SLM may be used to project the holographic image of the computer model of the 3D biological material. The SLM may be used to modulate the phase of light of a point-cloud representation or a lines-based representation of a computer model of the 3D biological material. The SLM may be used to modulate the phase of light of the holographic image of the computer model of the 3D biological material.

Projection of multi-photon excitation in three dimensions may also be achieved with the use of a dual digital micromirror device (DMD) system alone or in combination with a spatial light modulator (SLM). A pair of DMDs may be used with a pair of SLMs to print a 3D material using the methods described herein. At least one SLM and at least one DMD may be used to print a 3D material using the methods described herein. A pair of SLMs may be used to print a 3D material using the methods described herein. A pair of DMDs may be used to print a 3D material using the methods described herein. At least one SLM may be used to print a 3D material using the methods described herein. At least one DMD may be used to print a 3D material using the methods described herein. A DMD is an electrical input, optical output micro-electrical-mechanical system (MEMS) that allows for high speed, efficient, and reliable spatial light modulation. A DMD may comprise a plurality of microscopic mirrors (usually in the order of hundreds of thousands or millions) arranged in a rectangular array. Each microscopic mirror in a DMD may correspond to a pixel of the image to be displayed and can be rotated about e.g., 10-12° to an “on” or “off” state. In the “on” state, light from a projector bulb can be reflected into the microscopic mirror making its corresponding pixel appear bright on a screen. In the “off” state, the light can be directed elsewhere (usually onto a heatsink), making the microscopic mirror's corresponding pixel appear dark. The microscopic mirrors in a DMD may be composed of highly reflective aluminum and their length across is approximately 16 micrometers (μm). Each microscopic mirror may be built on top of an associated semiconductor memory cell and mounted onto a yoke which in turn is connected to a pair of support posts via torsion hinges. The degree of motion of each microscopic mirror may be controlled by loading each underlying semiconductor memory cell with a “1” or a “0.” Next, a voltage is applied, which may cause each microscopic mirror to be electrostatically deflected about the torsion hinge to the associated +/−degree state via electrostatic attraction.

With reference to FIGS. 3A-3C, the addition of an optional beam expander followed by a Bessel beam generating lens that is either a fixed axicon or a tunable acoustic gradient (TAG) lens may be added to alter the properties of the laser to achieve higher resolution and greater tissue printing depth, particularly in turbid solutions. The laser line, which may include the optional beam expander and/or Bessel beam generating lens, is directed with fast switch mirrors to distinct projection systems that have material advantages in the formation of specific structures associated with tissue printing. In some cases, a high resolution DMD mirror in conjunction with an SLM system may achieve higher axial resolution than is capable with two SLM systems. Finally, a laser line may be used with a single DMD or SLM system in conjunction with a mirror to allow for scan-less projection of a two-dimensional image in any of the axial planes. A 3D projection pattern may also be raster-scanned across a larger field of view by scan mirrors where in laser emission patterns, wavelength, and or power is controlled to match the raster scan speed such that a cohesive and complex structure may be deposited. Within the system containing more than one laser line the configurations may be any combination of dual SLM, dual DMD, single SLM, single DMD or simple planar scanning.

In some cases, one or more light paths, such as the ones shown in FIGS. 3A-3C, may be used independently or in concert. The lenses, gratings, and mirrors that focus and distribute the light or energy beam within the optical path may be placed between the primary, wave-front shaping elements necessary to distribute the light through key elements or modulate incoming light in the case of a grating, as described in FIG. 3A. At least one grating or mirror may be placed between wave-front shaping elements “F” (i.e., between an SLM, a DMD, and/or a TAG lens) for the purpose of focusing, distributing, or clipping the input laser light. The optical wave-front shaping device F may comprise an SLM, an LCOS-SLM, a DMD, a TAG lens, or any combination thereof.

In some cases, a DMD may be used to print a 3D biological material. The DMD may be used to project a point-cloud representation or a lines-based representation of a computer model of the 3D biological material. The methods disclosed herein may comprise converting the point-cloud representation or lines-based representation into a holographic image. The DMD may be used to project the holographic image of the computer model of the 3D biological material. The DMD may be used to print the 3D biological material.

In some cases, a combination of at least one SLM and at least one DMD may be used in the methods disclosed herein to print the 3D biological material. The combination of at least one SLM and at least one DMD may be arranged in series. The combination of at least one SLM and at least one DMD may be arranged in parallel. The combination of any number of SLMs and any number of DMDs may be arranged in series when used to print the 3D biological material. The combination of any number of SLMs and any number of DMDs may be arranged in parallel when used to print the 3D biological material.

The combination of at least two SLMs and at least one DMD may be used to print the 3D biological material. The combination of at least three SLMs and at least one DMD may be used to print the 3D biological material. The combination of at least four SLMs and at least one DMD may be used to print the 3D biological material. The combination of at least five SLMs and at least one DMD may be used to print the 3D biological material. The combination of at least ten SLMs and at least one DMD may be used to print the 3D biological material. The combination of at least twenty SLMs and at least one DMD may be used to print the 3D biological material.

The combination of at least one SLM and at least two DMDs may be used to print the 3D biological material. The combination of at least one SLM and at least three DMDs may be used to print the 3D biological material. The combination of at least one SLM and at least four DMDs may be used to print the 3D biological material. The combination of at least one SLM and at least five DMDs may be used to print the 3D biological material. The combination of at least one SLM and at least ten DMDs may be used to print the 3D biological material. The combination of at least one SLM and at least twenty DMDs may be used to print the 3D biological material.

The combination of at least two SLMs and at least two DMDs may be used to print the 3D biological material. The combination of at least three SLMs and at least three DMDs may be used to print the 3D biological material. The combination of at least four SLMs and at least four DMDs may be used to print the 3D biological material. The combination of at least five SLMs and at least five DMDs may be used to print the 3D biological material. The combination of at least ten SLMs and at least ten DMDs may be used to print the 3D biological material. The combination of at least twenty SLMs and at least twenty DMDs may be used to print the 3D biological material.

A liquid crystal SLM may be used to print the 3D biological material. A plurality of SLMs may be used to print the 3D biological material. The plurality of SLMs can be arranged in series. The plurality of SLMs can be arranged in parallel. At least one or more SLMs may be used to print the 3D biological material. At least two or more SLMs may be used to print the 3D biological material. At least three or more SLMs may be used to print the 3D biological material. At least four or more SLMs may be used to print the 3D biological material. At least five or more SLMs may be used to print the 3D biological material. At least ten or more SLMs may be used to print the 3D biological material. At least twenty or more SLMs may be used to print the 3D biological material. At least one to about fifty or more SLMs may be used to print the 3D biological material. At least one to about twenty or more SLMs may be used to print the 3D biological material. At least one to about fifteen or more SLMs may be used to print the 3D biological material. At least one to about ten or more SLMs may be used to print the 3D biological material. At least one to about five or more SLMs may be used to print the 3D biological material.

A plurality of DMDs may be used to print the 3D biological material. The plurality of DMDs can be arranged in series. The plurality of DMDs can be arranged in parallel. At least one or more DMDs may be used to print the 3D biological material. At least two or more DMDs may be used to print the 3D biological material. At least three or more DMDs may be used to print the 3D biological material. At least four or more DMDs may be used to print the 3D biological material. At least five or more DMDs may be used to print the 3D biological material. At least ten or more DMDs may be used to print the 3D biological material. At least twenty or more DMDs may be used to print the 3D biological material. At least one to about fifty or more DMDs may be used to print the 3D biological material. At least one to about twenty or more DMDs may be used to print the 3D biological material. At least one to about fifteen or more DMDs may be used to print the 3D biological material. At least one to about ten or more DMDs may be used to print the 3D biological material. At least one to about five or more DMDs may be used to print the 3D biological material.

In this design, SLM may refer to liquid crystal SLM and the function of the DMD may be similar to the SLM. These lasers may be controlled by one or more computer inputs to address location and print timing of multiple laser lines. An example overall design for the light path, including optional in-series excitations paths is illustrated in FIG. 3A along with further description of the elements provided in Table 1. Because of the extensive pulse-width between packets of two photon excitation light, any combination of these laser lines, which may be non-interfering, may be used simultaneously for printing and printing with simultaneous imaging. This may permit the interference between the beams to be substantially low such that the beams to not intersect. Therefore, the use of multiple laser lines with minimal to no interference is possible as illustrated in FIGS. 3B-3C along with further description of the elements also provided in Table 1. The group delay dispersion optical element in this configuration may be used to disperse two-photon packets such that the peak power output does not damage a fiber optic cable if one is to be used in certain configurations. In addition, group delay dispersion can concentrate photons into shorter pulse-widths such that more energy is imparted at the focal point or in the projected image allowing for more rapid printing.

Two photon excitation pulses may be temporally controlled such that excitation at a single spot occurs with pulses that are femto- to nanosecond range in length (dependent on laser tuning) while the timing between these photon packets is three to six orders of magnitude longer than the pulse width. This may allow for minimal cross-path interference of laser excitations making use of multiple lasers for simultaneous printing possible when using multiple laser lines in series. An example of multiple laser projections at three different theoretical wavelengths for the purpose of structure deposition is presented in FIG. 3B. Multi-photon lasers are tunable; thus, they may allow for a range of wavelengths to be selected. This is advantageous in tissue printing wherein different photoinitiators for polymerization that respond to different wavelengths may be used in combination or in series to prevent unwanted polymerization of left-over materials. Therefore, each of these laser lines may be tuned to a different multi-photon output wavelength, may have different peak power output, and may project a different element of the CAD image that comprises the tissue structure.

TABLE 1
Element descriptions for FIGS. 3A-3C

| Element | |
|---|---|
| Label | Description |
| 140a-c | Laser source. A first laser source 140a, a second |
| | laser source 140b, and a third laser |
| | source 140c may be a tunable multi-photon |
| | (femto-second pulsed) laser of a given |
| | power (e.g., between 1 and 50 watts and 640 to |
| | 1500 nm wavelength output). Femto- |
| | second laser sources may be tunable by computer |
| | software interaction and thus may |
| | be set to various wavelengths before or during |
| | the printing process to produce |
| | different excitation wavelengths. Optionally, |
| | the systems disclosed herein may have a |
| | pump laser system. |
| A | Mirror. A mirror with or without an infrared |
| | (IR) specific coating to improve |
| | reflectance. IR specific coating examples |
| | may include protected gold or protected |
| | silver based coatings. As shown in FIG. 3A, |
| | grating and/or mirrors may be added |
| | between elements “F” (i.e., between |
| | DMDs, SLMs, or TAG lenses). |
| B | Beam expander. An optional beam expander |
| | to expand the area of the laser pulse |
| | prior to projection by the DMD or SLM systems. |
| C | Axicon or TAG lens. In some tissue printing |
| | applications, the use of a Bessel beam |
| | may allow for improved or even power output |
| | at greater depths in hydrogels, media, |
| | or already printed structures. To produce a |
| | Bessel beam, an axicon which produces a |
| | fixed Bessel beam or tunable acoustic |
| | gradient lens (TAG), may produce a Bessel |
| | beam that is tunable and can be altered by |
| | altering an electric signal input. In the |
| | instance that a TAG lens is used, the input |
| | signal may be controlled by integrated |
| | computer software. |
| D | Dispersion compensation unit. The purpose of |
| | the dispersion compensation unit in |
| | this design is to concentrate emitted two-photon |
| | packets such that the peak power |
| | output is higher at the excitation point. This |
| | allows for improved polymerization as a |
| | result of improved peak power output at |
| | a specific wavelength. |
| E | Beam Dump. Beam dump allows for collection |
| | of stray laser light. |
| F | DMD, SLM, or TAG lens. In this example |
| | design, a DMD or SLM may be used to |
| | create an x, y plane of projection with a |
| | specific pattern of light that may be used to |
| | polymerize the monomers into structures or |
| | nets that contain cells. The addition of |
| | the second DMD or SLM may allow for |
| | projection of the x, y plane in the z or axial |
| | direction for three-dimensional holographic |
| | projection of the multiphoton excitation |
| | into the print vessel. This may allow for |
| | polymerization of the structures in three |
| | dimensions wherein all x, y, and z dimension |
| | features are deposited at the same time. |
| | Each DMD or SLM may be controlled by |
| | computer input and may be directed to |
| | project a specific CAD image or portion of |
| | a CAD image. Having the SLM or DMDs |
| | in series may allow for images to be |
| | projected simultaneously in different |
| | wavelengths of light in the case of multiple |
| | laser excitation sources (such as |
| | illustrated in FIG. 3B) or in the case of |
| | multiple repeating pattern projection SLMs or |
| | DMDs can be used to project different aspects |
| | of the same tissue without needing to |
| | switch the computer input, instead mirrors |
| | can be used to re-direct or turn ‘off’ or |
| | ‘on’ a particular light path and produce a |
| | given fixed structure associated with laser |
| | light paths 1, 2, 3, or 4. In cases where the |
| | Bessel beam is removed (element C), this |
| | may allow for different axial accuracies |
| | in printing a particular given structure. |
| | Therefore, certain elements of tissue |
| | structure may be better printed by different light |
| | paths. Rapid switching between laser |
| | light paths can allow for printing and |
| | polymerization to continue while an |
| | SLM or DMD series is re-programed for |
| | projection of the next tissue structure |
| | in a given series of printing steps. In some |
| | cases, element “F” can represent a TAG lens. |
| | The TAG lens as used as element “F” |
| | can manipulate light. The TAG lens as used |
| | as element “F” can holographically |
| | distribute light. |
| G | Movable mirror. A mirror with or without |
| | an IR specific coating to improve |
| | reflectance. IR specific coating examples |
| | may include protected gold or protected |
| | silver based coatings. These mirrors can |
| | be moveable and can be adjusted to be in an |
| | ‘on’ or ‘off’ state to redirect the laser |
| | light path through the printing system as |
| | desired. Control of mirror positioning |
| | may be dictated by computer software. |
| H | Beam combiner. Beam combiner allowing |
| | for multiple light paths to be recombined |
| | for simultaneous printing at different |
| | wavelengths. In FIG. 3B these may also be |
| | movable mirrors (G) that can allow for the |
| | same wavelengths to be printed with timed |
| | on/off states of the mirrors G. |
| I | Light path to the optics housing. |
| J | Band pass filter. The purpose of an optional |
| | band-pass filter may be to select a |
| | specific wavelength to be used in materials |
| | polymerization. Multi-photon excitation |
| | may have an emission spread that can span |
| | several tens of nanometers potentially |
| | leading to overlap in absorption and thus |
| | polymerization of materials with otherwise |
| | distinct absorption peaks. By selecting for |
| | specific wavelengths using a band pass |
| | filter the wavelength leading to |
| | polymerization may be fine-tuned to prevent |
| | undesirable cross-over effects when |
| | two different monomers with different |
| | responsiveness used in the same formulation. |
| K | Scan head. Two mirrors that represent optional |
| | laser light scanning or sintering |
| | in the x, y plane. These mirrors may vibrate |
| | at a given frequency, for example 20 |
| | kHz, one in the x-direction reflecting to the |
| | next mirror which may scan in the y |
| | direction. This scanning may create a plane |
| | of light that can be used to image tissues |
| | or polymerized units before, after, and |
| | during the polymerization process. This is |
| | possible as collagen and many other ordered |
| | structures can emit light via a non-linear |
| | process call second harmonic generation |
| | when polymerized but not when in a |
| | monomeric state. Therefore, using an |
| | additional excitation source tuned to a |
| | wavelength that may allow for second |
| | harmonic generation and imaging while not |
| | polymerizing the biomaterials can be |
| | useful for monitoring the printing process. |
| L | Long pass Mirror: A long pass mirror may |
| | allow multi-photon excitation from light |
| | path number 4 to pass through while |
| | reflecting any emission from a sample while in |
| | imaging mode (requires engagement |
| | of laser light path 4) to the series of |
| | photomultiplier tubes (PMT) M detectors |
| | and long pass or band pass mirrors of |
| | various wavelengths that may allow for |
| | specific emission wavelengths to be reflected |
| | into the PMTs for image collection via |
| | personal computer (PC) (i.e., computer |
| | processor) and appropriate imaging |
| | processing software. |
| M | Photo multiplier tubes. PMTs may be used |
| | in collection of images in microscopy. |
| N | Objective. This objective may serve the |
| | purpose of concentrating the multiphoton |
| | excitation such that polymerization of |
| | monomers to match the projected image may |
| | take place. |
| O | Movable long pass mirror. In instances |
| | where imaging may be performed with light |
| | path #4 the mirror 0 may be moved via |
| | software control to allow for laser light path |
| | 4 to enter the objective (N). In some |
| | incarnations light path 4 may be tuned to a |
| | distinct wavelength from laser light paths |
| | 1, 2, or 3 allowing for a long or short pass |
| | mirror or beam combiner to be used in |
| | place of O. |
| 1 | Laser light path 1 may be used to by-pass |
| | the beam expansion or beam expansion |
| | plus Bessel beam lens combination in |
| | favor of direct transmittance into the |
| | SLM/DMD series or individual SLM or DMD. |
| | Laser line one may also be redirected |
| | into laser line 5 which creates a single two |
| | photon pinpoint excitation, which may be |
| | used in optics housing alignment or raster |
| | scanning of a sample for imaging purposes. |
| 2 & 5 | Laser light path 2 may be transmitted |
| | through an optional beam expander and |
| | optional Bessel beam creating lens (axicon |
| | or TAG lens) then a single SLM or DMD |
| | and may also be re-directed to laser light path 5. |
| 3 & 4 | Laser light paths 3 and 4 may be passed |
| | through an optional beam expander and |
| | optional Bessel beam creating lens (axicon |
| | or TAG lens) followed by a combination |
| | of SLM or DMDs in series. Two distinct |
| | laser lines may allow for construction of |
| | dual SLM, dual DMD or a combination of |
| | the two which can increase flexibility in |
| | printing different sizes and types of |
| | structures. Furthermore, the laser line can be |
| | flickered between two different structures |
| | projected by each series to allow for near- |
| | simultaneous printing of complex structures |
| | that may not otherwise be achieved with |
| | a single DMD or SLM series. At any time |
| | these laser lines may be re-directed to the |
| | beam dump E which functions as a default |
| | off state. |

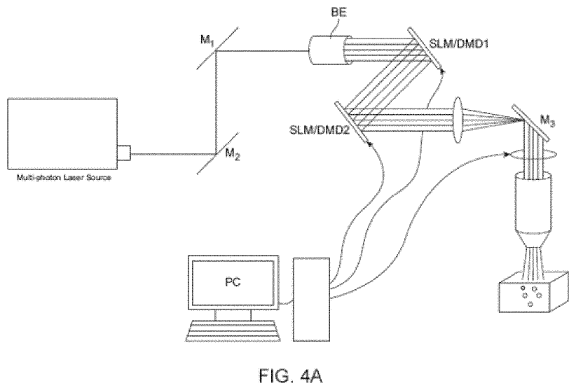

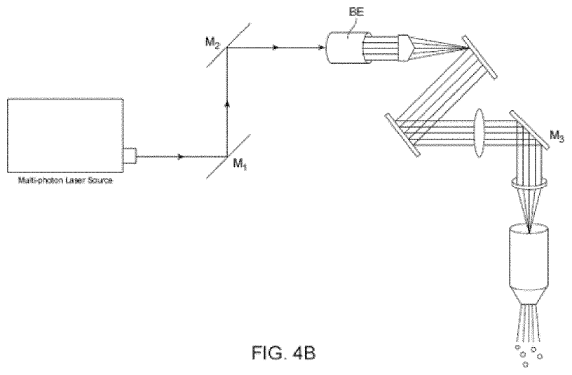

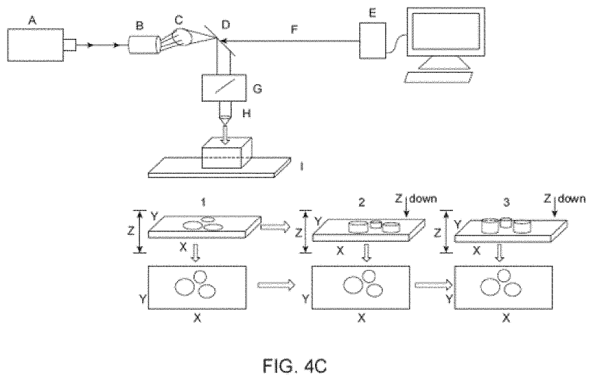

FIGS. 4A-4B demonstrates the placement of an optional beam expander prior to the axicon or tunable acoustic gradient (TAG) lens. This may allow for generation of a Bessel beam for the purpose of increased depth penetration in tissues and turbid media during printing without loss of focus fidelity. This feature may improve depth of printing through turbid media or through already formed tissues without loss of power.

A lens may be used to either widen or pre-focus the laser after the dual SLM or DMD combination. In addition, a laser attenuation device or filtering wheel that is computer controlled may be added prior to focusing optics to control the laser power output at the site of printing.

FIG. 4C illustrates a laser source A projecting a laser beam onto a beam collector B. Upon exiting the beam collector B, the laser beam may be directed to an optical TAG or axicon C and further to a movable, single SLM or DMD D for 2D x, y sheet projection for collagen net printing around cells and resultant structures printed with given Z-steps. The laser beam may be directed from the SLM or DMD D into a mirror G and then reflected onto the print head optics H. In this example, a two-dimensional (2D) projection may be created with a single SLM with a z-motor-stepped movement that matches the frame rate of the projection. Two-dimensional video projection of the z-stack slice may be achieved with a single DMD or a single SLM that is timed with z-movement such that each step projects a distinct image printing a 2D image from the top down. In another embodiment, a complex structure may be projected from the side, bottom up, or a different articulation and slice by slice, 2D projected and printed using either multi-photon or alternative laser excitation source. The source of CAD images F may be directed from the computer E into the system. The system may comprise a motorized stage I that may match the step rate (millisecond to second) and the step size of a Z-projection. The step size may be in the order of microns to nanometers. In FIG. 4C, 1, 2, and 3 illustrate examples of planar projection build steps.

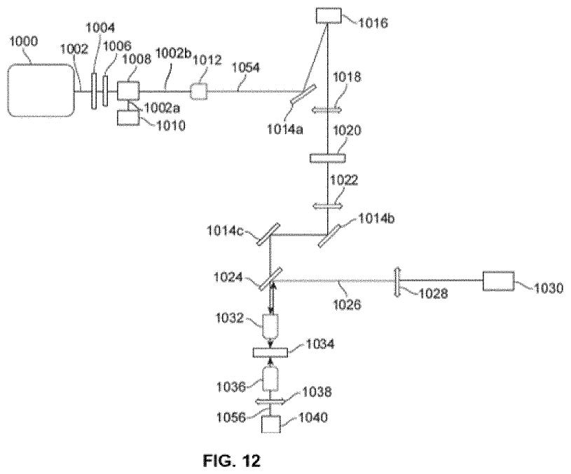

FIG. 12 illustrates the optical components and the optical path of an embodiment of the three-dimensional printing system. The optical components and the optical path shown in FIG. 12 may provide a three-dimensional printing system that may not use temporal focusing. The three-dimensional printing system may comprise an energy source 1000. The energy source 1000 may be a coherent light source. The energy source 1000 may be a laser light. The energy source 1000 may be a femto-second pulsed laser light source. The energy source 1000 may be a first laser source 140a, a second laser source 140b, or a third laser source 140c. The energy source 1000 may be a multi-photon laser beam 120. The energy source 1000 may be a two-photon laser beam. The energy source 1000 may be controlled by a computer system 1101. The energy source 1000 may be tuned by a computer system 1101. The computer system 1101 may control and/or set the energy wavelength of the energy source 1000 prior to or during the printing process. They computer system 1101 may produce different excitation wavelengths by setting the wavelength of the energy source 1000.

The energy source 1000 may be pulsed. The energy source 1000 may be pulsed at a rate of about 500 kilohertz (kHz). The energy source 1000 (e.g., laser) may provide energy (e.g., laser beam) having energy packets with pulsed energies (per packet) from about at least 1 micro joule (μJ) to 1,000,000 μJ. The energy source 1000 (e.g., laser) may provide energy (e.g., laser beam) having energy packets with pulsed energies (per packet) from about at least 1 micro joule (μJ) to 100,000 μJ or more. The energy source 1000 (e.g., laser) may provide energy (e.g., laser beam) having energy packets with pulsed energies (per packet) from about at least 1 micro joule (μJ) to 1,000 μJ or more. The energy source 1000 (e.g., laser) may provide energy (e.g., laser beam) having energy packets with pulsed energies (per packet) from about at least 1 micro joule (μJ) to 100 μJ or more. The energy source 1000 (e.g., laser) may provide energy (e.g., laser beam) having energy packets with pulsed energies (per packet) from about at least 10 micro joule (μJ) to 100 μJ or more. The energy source 1000 (e.g., laser) may provide energy (e.g., laser beam) having energy packets with pulsed energies (per packet) from about at least 1 micro joule (μJ) to 50 μJ or more. The energy source 1000 (e.g., laser) may provide energy (e.g., laser beam) having energy packets with pulsed energies (per packet) from about at least 1 micro joule (μJ) to 20 μJ or more. The energy source 1000 (e.g., laser) may provide energy (e.g., laser beam) having energy packets with pulsed energies (per packet) from about at least 1 micro joule (μJ) to 50 μJ or more. The energy source 1000 (e.g., laser) may provide energy (e.g., laser beam) having energy packets with pulsed energies (per packet) from about at least 40 micro joule (μJ) to 80 μJ or more. The energy source 1000 (e.g., laser) may provide energy (e.g., laser beam) having energy packets with pulsed energies (per packet) from about at least 120 micro joule (μJ) to 160 μJ or more.

The energy source 1000 (e.g., laser) may provide energy (e.g., laser beam) having energy packets with pulsed energies (per packet) of about 10 μJ. The energy source 1000 (e.g., laser) may provide energy (e.g., laser beam) having energy packets with pulsed energies (per packet) of about 20 μJ. The energy source 1000 (e.g., laser) may provide energy (e.g., laser beam) having energy packets with pulsed energies (per packet) of about 30 μJ. The energy source 1000 (e.g., laser) may provide energy (e.g., laser beam) having energy packets with pulsed energies (per packet) of about 40 μJ. The energy source 1000 (e.g., laser) may provide energy (e.g., laser beam) having energy packets with pulsed energies (per packet) of about 50 μJ. The energy source 1000 (e.g., laser) may provide energy (e.g., laser beam) having energy packets with pulsed energies (per packet) of about 60 μJ. The energy source 1000 (e.g., laser) may provide energy (e.g., laser beam) having energy packets with pulsed energies (per packet) of about 70 μJ. The energy source 1000 (e.g., laser) may provide energy (e.g., laser beam) having energy packets with pulsed energies (per packet) of about 80 μJ. The energy source 1000 (e.g., laser) may provide energy (e.g., laser beam) having energy packets with pulsed energies (per packet) of about 90 μJ. The energy source 1000 (e.g., laser) may provide energy (e.g., laser beam) having energy packets with pulsed energies (per packet) of about 100 μJ. The energy source 1000 (e.g., laser) may provide energy (e.g., laser beam) having energy packets with pulsed energies (per packet) of about 110 μJ. The energy source 1000 (e.g., laser) may provide energy (e.g., laser beam) having energy packets with pulsed energies (per packet) of about 120 μJ. The energy source 1000 (e.g., laser) may provide energy (e.g., laser beam) having energy packets with pulsed energies (per packet) of about 130 μJ. The energy source 1000 (e.g., laser) may provide energy (e.g., laser beam) having energy packets with pulsed energies (per packet) of about 140 μJ. The energy source 1000 (e.g., laser) may provide energy (e.g., laser beam) having energy packets with pulsed energies (per packet) of about 150 μJ. The energy source 1000 (e.g., laser) may provide energy (e.g., laser beam) having energy packets with pulsed energies (per packet) of about 160 μJ. The energy source 1000 (e.g., laser) may provide energy (e.g., laser beam) having energy packets with pulsed energies (per packet) of about 170 μJ. The energy source 1000 (e.g., laser) may provide energy (e.g., laser beam) having energy packets with pulsed energies (per packet) of about 180 μJ. The energy source 1000 (e.g., laser) may provide energy (e.g., laser beam) having energy packets with pulsed energies (per packet) of about 190 μJ. The energy source 1000 (e.g., laser) may provide energy (e.g., laser beam) having energy packets with pulsed energies (per packet) of about 200 μJ. The energy source 1000 (e.g., laser) may provide energy (e.g., laser beam) having energy packets with pulsed energies (per packet) of about 20,000 μJ. The energy source 1000 (e.g., laser) may provide energy (e.g., laser beam) having energy packets with pulsed energies (per packet) of about 100,000 μJ. The energy source 1000 (e.g., laser) may provide energy (e.g., laser beam) having energy packets with pulsed energies (per packet) of about 1,000,000 μJ.

The energy source 1000 (e.g., laser) may provide an energy beam (e.g., light beam) having a wavelength from, e.g., about at least 300 nm to about 5 mm or more. The energy source 1000 (e.g., laser) may provide energy (e.g., laser beam) having a wavelength of about at least 600 to about 1500 nm or more. The energy source 1000 (e.g., laser) may provide energy (e.g., laser beam) having a wavelength from about at least 350 nm to about 1800 nm or more. The energy source 1000 (e.g., laser) may provide energy (e.g., laser beam) having a wavelength from about at least 1800 nm to about 5 mm or more. The energy source 1000 (e.g., laser) may provide energy (e.g., laser beam) having a wavelength of about 300 nm. The energy source 1000 (e.g., laser) may provide energy (e.g., laser beam) having a wavelength of about 400 nm. The energy source 1000 (e.g., laser) may provide energy (e.g., laser beam) having a wavelength of about 600 nm. The energy source 1000 (e.g., laser) may provide energy (e.g., laser beam) having a wavelength of about 700 nm. The energy source 1000 (e.g., laser) may provide energy (e.g., laser beam) having a wavelength of about 800 nm. The energy source 1000 (e.g., laser) may provide energy (e.g., laser beam) having a wavelength of about 900 nm. The energy source 1000 (e.g., laser) may provide energy (e.g., laser beam) having a wavelength of about 1000 nm. The energy source 1000 (e.g., laser) may provide energy (e.g., laser beam) having a wavelength of about 1100 nm. The energy source 1000 (e.g., laser) may provide energy (e.g., laser beam) having a wavelength of about 1200 nm. The energy source 1000 (e.g., laser) may provide energy (e.g., laser beam) having a wavelength of about 1300 nm. The energy source 1000 (e.g., laser) may provide energy (e.g., laser beam) having a wavelength of about 1400 nm. The energy source 1000 (e.g., laser) may provide energy (e.g., laser beam) having a wavelength of about 1500 nm. The energy source 1000 (e.g., laser) may provide energy (e.g., laser beam) having a wavelength of about 1600 nm. The energy source 1000 (e.g., laser) may provide energy (e.g., laser beam) having a wavelength of about 1700 nm. The energy source 1000 (e.g., laser) may provide energy (e.g., laser beam) having a wavelength of about 1800 nm. The energy source 1000 (e.g., laser) may provide energy (e.g., laser beam) having a wavelength of about 1900 nm. The energy source 1000 (e.g., laser) may provide energy (e.g., laser beam) having a wavelength of about 2000 nm. The energy source 1000 (e.g., laser) may provide energy (e.g., laser beam) having a wavelength of about 3000 nm. The energy source 1000 (e.g., laser) may provide energy (e.g., laser beam) having a wavelength of about 4000 nm. The energy source 1000 (e.g., laser) may provide energy (e.g., laser beam) having a wavelength of about 5000 nm.

As shown in FIG. 12, the energy source 1000 may project a laser beam 1002 through a shutter 1004. Once the laser beam 1002 exits the shutter 1004, the laser beam 1002 may be directed through a rotating half-wave plate 1006. Rotating half-wave plates may be transparent plates with a specific amount of birefringence that may be used mostly for manipulating the polarization state of light beams. Rotating half-wave plates may have a slow axis and a fast axis (i.e., two polarization directions), which may be both perpendicular to the direction of the laser beam 1002. The rotating half-wave plate 1006 may alter the polarization state of the laser beam 1002 such that the difference in phase delay between the two linear polarization directions is π. The difference in phase delay may correspond to a propagation phase shift over a distance of λ/2. Other types of wave plates may be utilized with the system disclosed herein; for example, a rotating quarter-wave plate may be used. The rotating half-wave plate 1006 may be a true zero-order wave plate, a low order wave plate, or a multiple-order wave plate. The rotating half-wave plate 1006 may be composed of crystalline quartz (SiO₂), calcite (CaCO₃), magnesium fluoride (MgF₂), sapphire (Al₂O₃), mica, or a birefringent polymer.

The laser beam 1002 may exit the rotating half-wave plate 1006 and may be directed through a polarizing beam splitter 1008. The polarizing beam splitter 1008 may split the laser beam 1002 into a first laser beam 1002a and a second laser beam 1002b. The first laser beam 1002a may be directed to a beam dump 1010. The beam dump 1010 is an optical element that may be used to absorb stray portions of a laser beam. The beam dump 1010 may absorb the first laser beam 1002a. The first laser beam 1002a may be a stray laser beam. The beam dump 1010 may absorb the second laser beam 1002b. The second laser beam 1002b may be a stray laser beam. The laser beam 1002 may be directed into the beam dump 1010 in its entirety and thus, may serve as a default “off” state of the printing system. The second laser beam 1002b may be directed to a beam expander 1012. The beam expander 1012 may expand the size of the laser beam 1002b. The beam expander 1012 may increase the diameter of the input second laser beam 1002b to a larger diameter of an output, expanded laser beam 1054. The beam expander 1012 may be a prismatic beam expander. The beam expander 1012 may be a telescopic beam expander. The beam expander 1012 may be a multi-prism beam expander. The beam expander 1012 may be a Galilean beam expander. The beam expander 1012 may provide a beam expander power of about 2×, 3×, 5×, 10×, 20×, or 40×. The beam expander 1012 may provide a beam expander power ranging from about 2× to about 5×. The beam expander 1012 may provide continuous beam expansion between about 2× and about 5×. The beam expander 1012 may provide a beam expander power ranging from about 5× to about 10×. The beam expander 1012 may provide continuous beam expansion between about 5× and about 10×. The expanded laser beam 1054 may be collimated upon exiting the beam expander 1012.

After exiting the beam expander 1012, the expanded laser beam 1054 may be directed to a first minor 1014a, which may re-direct the expanded laser beam 1054 to a spatial light modulator (SLM) 1016. The SLM 1016 may be controlled by a computer system 1101. The SLM 1016 may be directed to project a specific image or a specific portion of an image of a material to be printed using the methods and systems disclosed herein. The material to be printed may be a biological material. The biological material may be a three-dimensional biological material. The specific image or the specific portion of the image may be one-dimensional, two-dimensional, and/or three-dimensional. The SLM 1016 may be directed to project at least one image simultaneously in different wavelengths of light. The SLM 1016 may be directed to project different aspects of the material to be printed with the use of minors instead of with the use of a computer system 1101. In some cases, at least one mirror may be used to re-direct or turn “off” or “on” a particular light path or laser beam in order to print different aspects or portions of the material to be printed.

After exiting the SLM 1016, the expanded laser beam 1054 may be directed to an f1 lens 1018. The f1 lens 1018 may be a focusing lens. After exiting the f1 lens 1018, the expanded laser beam 1054 may be directed to blocking element 1020. The blocking element 1020 may be immovable. The blocking element 1020 may suppress illumination from a zero-order spot. A zero-order may be a part of the energy from the expanded laser beam 1054 that is not diffracted and behaves according to the laws or reflection and refraction. After exiting the blocking element 1020, the expanded energy beam 1054 may be directed through an f2 lens 1022. The f2 lens may be a focusing lens.

After exiting the f2 lens 1022, the expanded laser beam 1054 may be directed onto a second minor 1014b and may be subsequently directed onto a third mirror 1014c. The third mirror 1014c may re-direct the expanded laser beam 1054 through a long pass dichroic minor 1024. The first mirror 1014a, the second mirror 1014b, and/or the third mirror 1014c may comprise an infrared (IR) coating to improve reflectance. The first minor 1014a, the second mirror 1014b, and/or the third mirror 1014c may not comprise an infrared (IR) coating. Non-limiting examples of IR coatings include protected gold-based coatings and protected silver-based coatings. The first mirror 1014a, the second mirror 1014b, and/or the third mirror 1014c may be controlled with a computer system 1101. The computer system 1101 may turn the first mirror 1014a, the second mirror 1014b, and/or the third mirror 1014c “on” or “off” in order to re-direct the expanded laser beam 1054 as desired.

The dichroic mirror may be a short pass dichroic minor. The long pass dichroic mirror 1024 may reflect the expanded laser beam 1054 into the focusing objective 1032. In some instances, a beam combiner may be used to re-direct the expanded laser beam 1054 into the focusing objective 1032 instead of using the long pass dichroic minor 1024. The long pass dichroic mirror 1024 may be controlled with a computer system 1101 to re-direct the expanded laser beam 1054 into the focusing objective 1032. The focusing objective 1032 may concentrate the expanded laser beam 1054 as it is projected into the printing chamber 1034. The printing chamber 1034 may be a media chamber 122. The printing chamber 1034 may comprise a cell-containing medium, a plurality of cells, cell constituents (e.g., organelles), and/or at least one polymer precursor.

A light-emitting diode (LED) collimator 1040 may be used as a source of collimated LED light 1056. The LED collimator 1040 may comprise a collimating lens and an LED emitter. The LED may be an inorganic LED, a high brightness LED, a quantum dot LED, or an organic LED. The LED may be a single color LED, a bi-color LED, or a tri-color LED. The LED may be a blue LED, an ultraviolet LED, a white LED, an infrared LED, a red LED, an orange LED, a yellow LED, a green LED, a violet LED, a pink LED, or a purple LED. The LED collimator 1040 may project a beam of collimated LED light 1056 through an f4 lens 1038. The f4 lens 1038 may be a focusing lens. Once the collimated LED light 1056 is transmitted through the f4 lens 1038, the collimated LED light 1056 may be directed into a light focusing objective 1036. The light focusing objective 1036 may focus the collimated LED light 1056 into the printing chamber 1034. The light focusing objective 1036 may focus the collimated LED light 1056 in the sample medium. The light focusing objective 1036 may focus the collimated LED light 1056 in the cell-containing medium. The collimated LED light 1056 may be transmitted through the printing chamber 1034 and into the focusing objective 1032. Once the collimated LED light 1056 exits the focusing objective 1032, the collimated LED light 1056 may be directed onto the long pass dichroic mirror 1024. The collimated LED light 1056 that is reflected off of the long pass dichroic mirror 1024 may be the sample emission 1026. The long pass dichroic minor 1024 may re-direct the sample emission 1026 into an f3 lens 1028. The f3 lens 1028 may be a focusing lens. Once sample emission 1026 is transmitted through the f3 lens 1028, a detection system 1030 detects and/or collects the sample emission 1026 for imaging. The detection system 1030 may comprise at least one photomultiplier tube (PMT). The detection system 1030 may comprise at least one camera. The camera may be a complementary metal-oxide semiconductor (CMOS) camera, a scientific CMOS camera, a charge-coupled device (CCD) camera, or an electron-multiplying charge-coupled device (EM-CCD). The detection system 1030 may comprise at least one array-based detector.

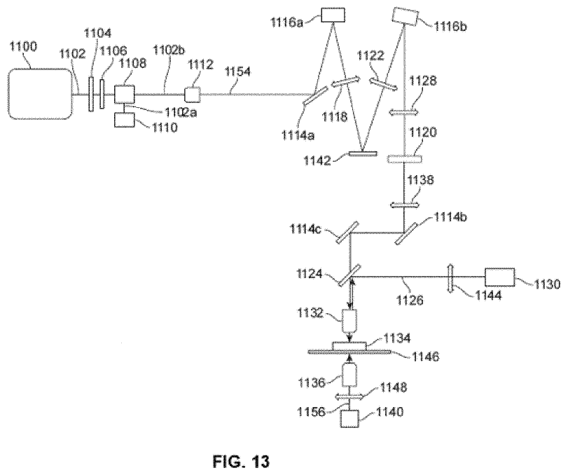

FIG. 13 illustrates the optical components and the optical path of yet another embodiment of the three-dimensional printing system. The optical components and the optical path shown in FIG. 13 provide a three-dimensional printing system that may use temporal focusing. The three-dimensional printing system may comprise an energy source 1100. The energy source 1100 may be a coherent light source. The energy source 1100 may be a laser light. The energy source 1100 may be a femto-second pulsed laser light source. The energy source 1100 may be a first laser source 140a, a second laser source 140b, or a third laser source 140c. The energy source 1100 may be a multi-photon laser beam 120. The energy source 1100 may be a two-photon laser beam. The energy source 1100 may be controlled by a computer system 1101. The energy source 1100 may be tuned by a computer system 1101. The computer system 1101 may control and/or set the energy wavelength of the energy source 1100 prior to or during the printing process. They computer system 1101 may produce different excitation wavelengths by setting the wavelength of the energy source 1100.

The energy source 1100 may be pulsed. The energy source 1100 may be pulsed at a rate of about 500 kilohertz (kHz). The energy source 1100 (e.g., laser) may provide energy (e.g., laser beam) having energy packets with pulsed energies (per packet) from about at least 1 micro joule (μJ) to 1,000,000 μJ. The energy source 1100 (e.g., laser) may provide energy (e.g., laser beam) having energy packets with pulsed energies (per packet) from about at least 1 micro joule (μJ) to 100,000 μJ or more. The energy source 1100 (e.g., laser) may provide energy (e.g., laser beam) having energy packets with pulsed energies (per packet) from about at least 1 micro joule (μJ) to 1,000 μJ or more. The energy source 1100 (e.g., laser) may provide energy (e.g., laser beam) having energy packets with pulsed energies (per packet) from about at least 1 micro joule (μJ) to 100 μJ or more. The energy source 1100 (e.g., laser) may provide energy (e.g., laser beam) having energy packets with pulsed energies (per packet) from about at least 10 micro joule (μJ) to 100 μJ or more. The energy source 1100 (e.g., laser) may provide energy (e.g., laser beam) having energy packets with pulsed energies (per packet) from about at least 1 micro joule (μJ) to 50 μJ or more. The energy source 1100 (e.g., laser) may provide energy (e.g., laser beam) having energy packets with pulsed energies (per packet) from about at least 1 micro joule (μJ) to 20 μJ or more. The energy source 1100 (e.g., laser) may provide energy (e.g., laser beam) having energy packets with pulsed energies (per packet) from about at least 1 micro joule (μJ) to 50 μJ or more. The energy source 1100 (e.g., laser) may provide energy (e.g., laser beam) having energy packets with pulsed energies (per packet) from about at least 40 micro joule (μJ) to 80 μJ or more. The energy source 1100 (e.g., laser) may provide energy (e.g., laser beam) having energy packets with pulsed energies (per packet) from about at least 120 micro joule (μJ) to 160 μJ or more.

The energy source 1100 (e.g., laser) may provide energy (e.g., laser beam) having energy packets with pulsed energies (per packet) of about 10 μJ. The energy source 1100 (e.g., laser) may provide energy (e.g., laser beam) having energy packets with pulsed energies (per packet) of about 20 μJ. The energy source 1100 (e.g., laser) may provide energy (e.g., laser beam) having energy packets with pulsed energies (per packet) of about 30 μJ. The energy source 1100 (e.g., laser) may provide energy (e.g., laser beam) having energy packets with pulsed energies (per packet) of about 40 μJ. The energy source 1100 (e.g., laser) may provide energy (e.g., laser beam) having energy packets with pulsed energies (per packet) of about 50 μJ. The energy source 1100 (e.g., laser) may provide energy (e.g., laser beam) having energy packets with pulsed energies (per packet) of about 60 μJ. The energy source 1100 (e.g., laser) may provide energy (e.g., laser beam) having energy packets with pulsed energies (per packet) of about 70 μJ. The energy source 1100 (e.g., laser) may provide energy (e.g., laser beam) having energy packets with pulsed energies (per packet) of about 80 μJ. The energy source 1100 (e.g., laser) may provide energy (e.g., laser beam) having energy packets with pulsed energies (per packet) of about 90 μJ. The energy source 1100 (e.g., laser) may provide energy (e.g., laser beam) having energy packets with pulsed energies (per packet) of about 100 μJ. The energy source 1100 (e.g., laser) may provide energy (e.g., laser beam) having energy packets with pulsed energies (per packet) of about 110 μJ. The energy source 1100 (e.g., laser) may provide energy (e.g., laser beam) having energy packets with pulsed energies (per packet) of about 120 μJ. The energy source 1100 (e.g., laser) may provide energy (e.g., laser beam) having energy packets with pulsed energies (per packet) of about 130 μJ. The energy source 1100 (e.g., laser) may provide energy (e.g., laser beam) having energy packets with pulsed energies (per packet) of about 140 μJ. The energy source 1100 (e.g., laser) may provide energy (e.g., laser beam) having energy packets with pulsed energies (per packet) of about 150 μJ. The energy source 1100 (e.g., laser) may provide energy (e.g., laser beam) having energy packets with pulsed energies (per packet) of about 160 μJ. The energy source 1100 (e.g., laser) may provide energy (e.g., laser beam) having energy packets with pulsed energies (per packet) of about 170 μJ. The energy source 1100 (e.g., laser) may provide energy (e.g., laser beam) having energy packets with pulsed energies (per packet) of about 180 μJ. The energy source 1100 (e.g., laser) may provide energy (e.g., laser beam) having energy packets with pulsed energies (per packet) of about 190 μJ. The energy source 1100 (e.g., laser) may provide energy (e.g., laser beam) having energy packets with pulsed energies (per packet) of about 200 μJ. The energy source 1100 (e.g., laser) may provide energy (e.g., laser beam) having energy packets with pulsed energies (per packet) of about 20,000 μJ. The energy source 1100 (e.g., laser) may provide energy (e.g., laser beam) having energy packets with pulsed energies (per packet) of about 100,000 μJ. The energy source 1100 (e.g., laser) may provide energy (e.g., laser beam) having energy packets with pulsed energies (per packet).

The energy source 1100 (e.g., laser) may provide energy (e.g., laser beam) having a wavelength from about 300 nm to 5 mm, 600 nm to 1500 nm, 350 nm to 1800 nm, or 1800 nm to 5 mm. The energy source 1100 (e.g., laser) may provide energy (e.g., laser beam) having a wavelength of at least about 300 nm, 400 nm, 500 nm, 600 nm, 700 nm, 800 nm, 900 nm, 1 mm, 1.1 mm, 1.2, mm, 1.3 mm, 1.4 mm, 1.5 mm, 1.6 mm, 1.7 mm, 1.8 mm, 1.9 mm, 2 mm, 3 mm, 4 mm, 5 mm, or greater.

As shown in FIG. 13, the energy source 1100 may project a laser beam 1102 through a shutter 1104. Once the laser beam 1102 exits the shutter 1104, the laser beam 1102 may be directed through a rotating half-wave plate 1106. The rotating half-wave plate 1106 may alter the polarization state of the laser beam 1102 such that the difference in phase delay between the two linear polarization directions is π. The difference in phase delay may correspond to a propagation phase shift over a distance of 212. Other types of wave plates may be utilized with the system disclosed herein; for example, a rotating quarter-wave plate may be used. The rotating half-wave plate 1106 may be a true zero-order wave plate, a low order wave plate, or a multiple-order wave plate. The rotating half-wave plate 1106 may be composed of crystalline quartz (Sift), calcite (CaCO₃), magnesium fluoride (MgF₂), sapphire (Al₂O₃), mica, or a birefringent polymer.

The laser beam 1102 may exit the rotating half-wave plate 1106 and may be directed through a polarizing beam splitter 1108. The polarizing beam splitter 1108 may split the laser beam 1102 into a first laser beam 1102a and a second laser beam 1102b. The first laser beam 1102a may be directed to a beam dump 1110. The beam dump 1110 is an optical element that may be used to absorb stray portions of a laser beam. The beam dump 1110 may absorb the first laser beam 1102a. The first laser beam 1102a may be a stray laser beam. The beam dump 1110 may absorb the second laser beam 1102b. The second laser beam 1102b may be a stray laser beam. The laser beam 1102 may be directed into the beam dump 1110 in its entirety and thus, may serve as a default “off” state of the printing system. The second laser beam 1102b may be directed to a beam expander 1112. The beam expander 1112 may expand the size of the second laser beam 1102b. The beam expander 1112 may increase the diameter of the input, second laser beam 1102b to a larger diameter of an output, expanded laser beam 1154. The beam expander 1112 may be a prismatic beam expander. The beam expander 1112 may be a telescopic beam expander. The beam expander 1112 may be a multi-prism beam expander. The beam expander 1112 may be a Galilean beam expander. The beam expander 1112 may provide a beam expander power of about 2×, 3×, 5×, 10×, 20×, or 40×. The beam expander 1112 may provide a beam expander power ranging from about 2× to about 5×. The beam expander 1112 may provide continuous beam expansion between about 2× and about 5×. The beam expander 1112 may provide a beam expander power ranging from about 5× to about 10×. The beam expander 1112 may provide continuous beam expansion between about 5× and about 10×. The expanded laser beam 1154 may be collimated upon exiting the beam expander 1112.

After exiting the beam expander 1112, the expanded laser beam 1154 may be directed to a first mirror 1114a, which may re-direct the expanded laser beam 1154 to a first spatial light modulator (SLM) 1116a. After exiting the first SLM 1116, the expanded laser beam 1154 may be directed to an f1 lens 1118. The f1 lens 1118 may be a focusing lens. After exiting the f1 lens, the expanded laser beam 1154 may be directed to a grating 1142. The grating 1142 may be a diffractive laser beam splitter. The grating 1142 may be a holographic grating. The grating 1142 may be a ruled grating. The grating 1142 may be a subwavelength grating. The grating 1142 may split and/or diffract the expanded laser beam 1154 into a plurality of expanded laser beams (not shown in FIG. 13). The grating 1142 may act as a dispersive element. Once the expanded laser beam 1154 is split, diffracted, and/or dispersed by the grating 1142, the expanded laser beam 1154 may be transmitted through an f2 lens 1122. The f2 lens 1122 may be a focusing lens. After exiting the f2 lens 1122, the expanded laser beam 1154 may be directed to a second SLM 1116b. The SLMs (i.e., the first SLM 1116a and the second SLM 1116b) may be controlled by a computer system 1101. The SLMs may perform all of the functions, as described supra, of the SLM 1016 presented in FIG. 12.

After exiting the second SLM 1116b, the expanded laser beam 1154 may be directed to an f3 lens 1128. The f3 lens 1128 may be a focusing lens. After exiting the f3 lens, the expanded laser beam 1154 may be directed to blocking element 1120. The blocking element 1120 may be immovable. The blocking element 1120 may be used to suppress illumination from a zero-order spot. After exiting the blocking element 1120, the expanded energy beam 1154 may be directed through an f4 lens 1138. The f4 lens 1138 may be a focusing lens. After exiting the f4 lens 1138, the expanded laser beam 1154 may be directed onto a second mirror 1114b and may be subsequently directed onto a third mirror 1114c. The third mirror 1114c may re-direct the expanded laser beam 1154 through a long pass dichroic mirror 1124. The first mirror 1114a, the second mirror 1114b, and/or the third mirror 1114c may be controlled with a computer system 1101. The computer system 1101 may turn the first mirror 1114a, the second mirror 1114b, and/or the third mirror 1114c “on” or “off” in order to re-direct the expanded laser beam 1154 as desired. The dichroic mirror may be a short pass dichroic mirror. The long pass dichroic mirror 1124 may reflect the expanded laser beam 1154 into the focusing objective 1132. In some instances, a beam combiner may be used to re-direct the expanded laser beam 1154 into the focusing objective 1132 instead of using the long pass dichroic mirror 1124. The long pass dichroic mirror 1124 may be controlled with a computer system 1101 to re-direct the expanded laser beam 1154 into the focusing objective 1132. The focusing objective 1132 may concentrate the expanded laser beam 1154 as it is projected into the printing chamber 1134. The printing chamber 1134 may be a media chamber 122. The printing chamber 1134 may comprise a cell-containing medium, a plurality of cells, cell constituents (e.g., organelles), and/or at least one polymer precursor.

The printing chamber 1134 may be mounted on a movable stage 1146. The movable stage 1146 may be an xy stage, a z stage, and/or an xyz stage. The movable stage 1146 may be manually positioned. The movable stage 1146 may be automatically positioned. The movable stage 1146 may be a motorized stage. The movable stage 1146 may be controlled by the computer system 1101. The computer system 1101 may control the movement of the movable stage 1146 in the x, y, and/or z directions. The computer system 1101 may automatically position the movable stage 1146 in a desired x, y, and/or z position. The computer system 1101 may position the movable stage 1146 in a desired x, y, and/or z position with a positional accuracy of at most about 3 μm. The computer system 1101 may position the movable stage 1146 in a desired x, y, and/or z position with a positional accuracy of at most about 2 μm. The computer system 1101 may position the movable stage 1146 in a desired x, y, and/or z position with a positional accuracy of at most about 1 μm. The computer system 1101 may automatically adjust the position of the movable stage 1146 prior or during three-dimensional printing. The computer system 1101 may comprise a piezoelectric (piezo) controller to provide computer-controlled z-axis (i.e., vertical direction) positioning and active location feedback. The computer system 1101 may comprise a joystick console to enable a user to control a position of the movable stage 1146. The joystick console may be a z-axis console and/or an x-axis and y-axis console. The movable stage 1146 may comprise a printing chamber holder. The printing chamber holder may be a bracket, a clip, and/or a recessed sample holder. The movable stage 1146 may comprise a multi-slide holder, a slide holder, and/or a petri dish holder. The movable stage 1146 may comprise a sensor to provide location feedback. The sensor may be a capacitive sensor. The sensor may be a piezoresistive sensor. The movable stage 1146 may comprise at least one actuator (e.g., piezoelectric actuator) that moves (or positions) the movable stage 1146.

A light-emitting diode (LED) collimator 1140 may be used as a source of collimated LED light 1156. The LED collimator 1140 may comprise a collimating lens and an LED emitter. The LED may be an inorganic LED, a high brightness LED, a quantum dot LED, or an organic LED. The LED may be a single color LED, a bi-color LED, or a tri-color LED. The LED may be a blue LED, an ultraviolet LED, a white LED, an infrared LED, a red LED, an orange LED, a yellow LED, a green LED, a violet LED, a pink LED, or a purple LED. The LED collimator 1140 may project a beam of collimated LED light 1156 through an f6 lens 1148. The f6 lens 1148 may be a focusing lens. Once the collimated LED light 1156 is transmitted through the f6 lens 1148, the collimated LED light 1156 may be directed into a light focusing objective 1136. The light focusing objective 1136 may focus the collimated LED light 1156 into the printing chamber 1134. The light focusing objective 1136 may focus the collimated LED light 1156 in the sample medium. The light focusing objective 1136 may focus the collimated LED light 1156 in the cell-containing medium. The collimated LED light 1156 may be transmitted through the printing chamber 1134 and into the focusing objective 1132. Once the collimated LED light 1156 exits the focusing objective 1132, the collimated LED light 1156 may be directed onto the long pass dichroic mirror 1124. The collimated LED light 1156 that is reflected off of the long pass dichroic mirror 1124 may be the sample emission 1126. The long pass dichroic mirror 1124 may re-direct the sample emission 1126 into an f5 lens 1144. The f5 lens 1144 may be a focusing lens. Once sample emission 1126 is transmitted through the f5 lens 1144, a detection system 1130 detects and/or collects the sample emission 1126 for imaging. The detection system 1130 may comprise at least one photomultiplier tube (PMT). The detection system 1130 may comprise at least one camera. The camera may be a complementary metal-oxide semiconductor (CMOS) camera, a scientific CMOS camera, a charge-coupled device (CCD) camera, or an electron-multiplying charge-coupled device (EM-CCD). The detection system 1130 may comprise at least one array-based detector.

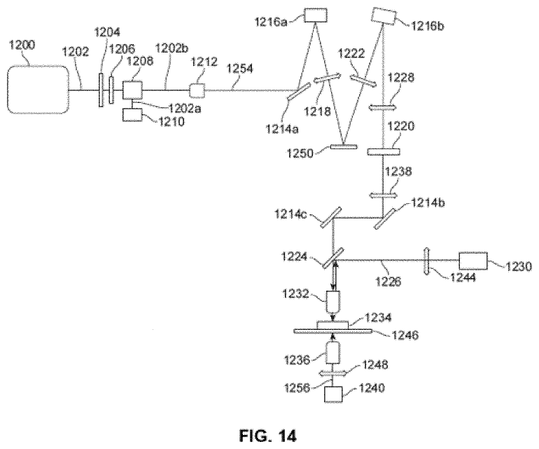

FIG. 14 illustrates the optical components and the optical path of an additional embodiment of the three-dimensional printing system. The optical components and the optical path shown in FIG. 14 provide a three-dimensional printing system that may not use temporal focusing. The three-dimensional printing system may comprise an energy source 1200. The energy source 1200 may be a coherent light source. The energy source 1200 may be a laser light. The energy source 1200 may be a femto-second pulsed laser light source. The energy source 1200 may be a first laser source 140a, a second laser source 140b, or a third laser source 140c. The energy source 1200 may be a multi-photon laser beam 120. The energy source 1200 may be controlled by a computer system 1101. The energy source 1200 may be tuned by a computer system 1101. The computer system 1101 may control and/or set the energy wavelength of the energy source 1200 prior to or during the printing process. They computer system 1101 may produce different excitation wavelengths by setting the wavelength of the energy source 1200.

The energy source 1200 may be pulsed. The energy source 1200 may be pulsed at a rate of about 500 kilohertz (kHz). The energy source 1200 (e.g., laser) may provide energy (e.g., laser beam) having energy packets with pulsed energies (per packet) from about at least 1 micro joule (μJ) to 1,000,000 μJ. The energy source 1200 (e.g., laser) may provide energy (e.g., laser beam) having energy packets with pulsed energies (per packet) from about at least 1 micro joule (μJ) to 100,000 μJ or more. The energy source 1200 (e.g., laser) may provide energy (e.g., laser beam) having energy packets with pulsed energies (per packet) from about at least 1 micro joule (μJ) to 1,000 μJ or more. The energy source 1200 (e.g., laser) may provide energy (e.g., laser beam) having energy packets with pulsed energies (per packet) from about at least 1 micro joule (μJ) to 100 μJ or more. The energy source 1200 (e.g., laser) may provide energy (e.g., laser beam) having energy packets with pulsed energies (per packet) from about at least 10 micro joule (μJ) to 100 μJ or more. The energy source 1200 (e.g., laser) may provide energy (e.g., laser beam) having energy packets with pulsed energies (per packet) from about at least 1 micro joule (μJ) to 50 μJ or more. The energy source 1200 (e.g., laser) may provide energy (e.g., laser beam) having energy packets with pulsed energies (per packet) from about at least 1 micro joule (μJ) to 20 μJ or more. The energy source 1200 (e.g., laser) may provide energy (e.g., laser beam) having energy packets with pulsed energies (per packet) from about at least 1 micro joule (μJ) to 50 μJ or more. The energy source 1200 (e.g., laser) may provide energy (e.g., laser beam) having energy packets with pulsed energies (per packet) from about at least 40 micro joule (μJ) to 80 μJ or more. The energy source 1200 (e.g., laser) may provide energy (e.g., laser beam) having energy packets with pulsed energies (per packet) from about at least 120 micro joule (μJ) to 160 μJ or more.

The energy source 1200 (e.g., laser) may provide energy (e.g., laser beam) having energy packets with pulsed energies (per packet) of about 10 μJ. The energy source 1200 (e.g., laser) may provide energy (e.g., laser beam) having energy packets with pulsed energies (per packet) of about 20 μJ. The energy source 1200 (e.g., laser) may provide energy (e.g., laser beam) having energy packets with pulsed energies (per packet) of about 30 μJ. The energy source 1200 (e.g., laser) may provide energy (e.g., laser beam) having energy packets with pulsed energies (per packet) of about 40 μJ. The energy source 1200 (e.g., laser) may provide energy (e.g., laser beam) having energy packets with pulsed energies (per packet) of about 50 μJ. The energy source 1200 (e.g., laser) may provide energy (e.g., laser beam) having energy packets with pulsed energies (per packet) of about 60 μJ. The energy source 1200 (e.g., laser) may provide energy (e.g., laser beam) having energy packets with pulsed energies (per packet) of about 70 μJ. The energy source 1200 (e.g., laser) may provide energy (e.g., laser beam) having energy packets with pulsed energies (per packet) of about 80 μJ. The energy source 1200 (e.g., laser) may provide energy (e.g., laser beam) having energy packets with pulsed energies (per packet) of about 90 μJ. The energy source 1200 (e.g., laser) may provide energy (e.g., laser beam) having energy packets with pulsed energies (per packet) of about 100 μJ. The energy source 1200 (e.g., laser) may provide energy (e.g., laser beam) having energy packets with pulsed energies (per packet) of about 110 μJ. The energy source 1200 (e.g., laser) may provide energy (e.g., laser beam) having energy packets with pulsed energies (per packet) of about 120 μJ. The energy source 1200 (e.g., laser) may provide energy (e.g., laser beam) having energy packets with pulsed energies (per packet) of about 130 μJ. The energy source 1200 (e.g., laser) may provide energy (e.g., laser beam) having energy packets with pulsed energies (per packet) of about 140 μJ. The energy source 1200 (e.g., laser) may provide energy (e.g., laser beam) having energy packets with pulsed energies (per packet) of about 150 μJ. The energy source 1200 (e.g., laser) may provide energy (e.g., laser beam) having energy packets with pulsed energies (per packet) of about 160 μJ. The energy source 1200 (e.g., laser) may provide energy (e.g., laser beam) having energy packets with pulsed energies (per packet) of about 170 μJ. The energy source 1200 (e.g., laser) may provide energy (e.g., laser beam) having energy packets with pulsed energies (per packet) of about 180 μJ. The energy source 1200 (e.g., laser) may provide energy (e.g., laser beam) having energy packets with pulsed energies (per packet) of about 190 μJ. The energy source 1200 (e.g., laser) may provide energy (e.g., laser beam) having energy packets with pulsed energies (per packet) of about 200 μJ. The energy source 1200 (e.g., laser) may provide energy (e.g., laser beam) having energy packets with pulsed energies (per packet) of about 20,000 μJ. The energy source 1200 (e.g., laser) may provide energy (e.g., laser beam) having energy packets with pulsed energies (per packet) of about 100,000 μJ. The energy source 1200 (e.g., laser) may provide energy (e.g., laser beam) having energy packets with pulsed energies (per packet).

The energy source 1200 (e.g., laser) may provide energy (e.g., laser beam) having a wavelength from, e.g., about at least 300 nm to about 5 mm or more. The energy source 1200 (e.g., laser) may provide energy (e.g., laser beam) having a wavelength of about at least 600 to about 1500 nm or more. The energy source 1200 (e.g., laser) may provide energy (e.g., laser beam) having a wavelength from about at least 350 nm to about 1800 nm or more. The energy source 1200 (e.g., laser) may provide energy (e.g., laser beam) having a wavelength from about at least 1800 nm to about 5 mm or more. The energy source 1200 (e.g., laser) may provide energy (e.g., laser beam) having a wavelength of about 300 nm. The energy source 1200 (e.g., laser) may provide energy (e.g., laser beam) having a wavelength of about 400 nm. The energy source 1200 (e.g., laser) may provide energy (e.g., laser beam) having a wavelength of about 600 nm. The energy source 1200 (e.g., laser) may provide energy (e.g., laser beam) having a wavelength of about 700 nm. The energy source 1200 (e.g., laser) may provide energy (e.g., laser beam) having a wavelength of about 800 nm. The energy source 1200 (e.g., laser) may provide energy (e.g., laser beam) having a wavelength of about 900 nm. The energy source 1200 (e.g., laser) may provide energy (e.g., laser beam) having a wavelength of about 1200 nm. The energy source 1200 (e.g., laser) may provide energy (e.g., laser beam) having a wavelength of about 1200 nm. The energy source 1200 (e.g., laser) may provide energy (e.g., laser beam) having a wavelength of about 1200 nm. The energy source 1200 (e.g., laser) may provide energy (e.g., laser beam) having a wavelength of about 1300 nm. The energy source 1200 (e.g., laser) may provide energy (e.g., laser beam) having a wavelength of about 1400 nm. The energy source 1200 (e.g., laser) may provide energy (e.g., laser beam) having a wavelength of about 1500 nm. The energy source 1200 (e.g., laser) may provide energy (e.g., laser beam) having a wavelength of about 1600 nm. The energy source 1200 (e.g., laser) may provide energy (e.g., laser beam) having a wavelength of about 1700 nm. The energy source 1200 (e.g., laser) may provide energy (e.g., laser beam) having a wavelength of about 1800 nm. The energy source 1200 (e.g., laser) may provide energy (e.g., laser beam) having a wavelength of about 1900 nm. The energy source 1200 (e.g., laser) may provide energy (e.g., laser beam) having a wavelength of about 2000 nm. The energy source 1200 (e.g., laser) may provide energy (e.g., laser beam) having a wavelength of about 3000 nm. The energy source 1200 (e.g., laser) may provide energy (e.g., laser beam) having a wavelength of about 4000 nm. The energy source 1200 (e.g., laser) may provide energy (e.g., laser beam) having a wavelength of about 5000 nm.

As shown in FIG. 14, the energy source 1200 may project a laser beam 1202 through a shutter 1104. Once the laser beam 1202 exits the shutter 1204, the laser beam 1202 may be directed through a rotating half-wave plate 1206. The rotating half-wave plate 1206 may alter the polarization state of the laser beam 1202 such that the difference in phase delay between the two linear polarization directions is π. The difference in phase delay may correspond to a propagation phase shift over a distance of 212. Other types of wave plates may be utilized with the system disclosed herein; for example, a rotating quarter-wave plate may be used. The rotating half-wave plate 1206 may be a true zero-order wave plate, a low order wave plate, or a multiple-order wave plate. The rotating half-wave plate 1206 may be composed of crystalline quartz (Sift), calcite (CaCO₃), magnesium fluoride (MgF₂), sapphire (Al₂O₃), mica, or a birefringent polymer.

The laser beam 1202 may exit the rotating half-wave plate 1206 and may be directed through a polarizing beam splitter 1208. The polarizing beam splitter 1208 may split the laser beam 1202 into a first laser beam 1202a and a second laser beam 1202b. The first laser beam 1202a may be directed to a beam dump 1210. The beam dump 1210 is an optical element that may be used to absorb stray portions of a laser beam. The beam dump 1210 may absorb the first laser beam 1202a. The first laser beam 1202a may be a stray laser beam. The beam dump 1210 may absorb the second laser beam 1202b. The second laser beam 1202b may be a stray laser beam. The laser beam 1202 may be directed into the beam dump 1210 in its entirety and thus, may serve as a default “off” state of the printing system. The second laser beam 1202b may be directed to a beam expander 1212. The beam expander 1212 may expand the size of the second laser beam 1202b. The beam expander 1212 may increase the diameter of the input, second laser beam 1202b to a larger diameter of an output, expanded laser beam 1254. The beam expander 1212 may be a prismatic beam expander. The beam expander 1212 may be a telescopic beam expander. The beam expander 1212 may be a multi-prism beam expander. The beam expander 1212 may be a Galilean beam expander. The beam expander 1212 may provide a beam expander power of about 2×, 3×, 5×, 10×, 20×, or 40×. The beam expander 1212 may provide a beam expander power ranging from about 2× to about 5×. The beam expander 1212 may provide continuous beam expansion between about 2× and about 5×. The beam expander 1212 may provide a beam expander power ranging from about 5× to about 10×. The beam expander 1212 may provide continuous beam expansion between about 5× and about 10×. The expanded laser beam 1254 may be collimated upon exiting the beam expander 1212.

After exiting the beam expander 1212, the expanded laser beam 1254 may be directed to a first mirror 1214a, which may re-direct the expanded laser beam 1254 to a first spatial light modulator (SLM) 1216a. After exiting the first SLM 1216, the expanded laser beam 1254 may be directed to an f1 lens 1218. The f1 lens 1218 may be a focusing lens. After exiting the f1 lens, the expanded laser beam 1254 may be directed to a mirror with blocking element 1250. The mirror with blocking element 1250 may be used to suppress illumination from a zero-order spot.

Once the expanded laser beam 1254 is reflected by the mirror with blocking element 1250, the expanded laser beam 1254 may be transmitted through an f2 lens 1222. The f2 lens 1222 may be a focusing lens. After exiting the f2 lens 1222, the expanded laser beam 1254 may be directed to a second SLM 1216b. The SLMs (i.e., the first SLM 1216a and the second SLM 1216b) may be controlled by a computer system 1101. The SLMs may perform all of the functions, as described supra, of the SLM 1016 and the SLM 1116, as presented in FIGS. 44 and 45, respectively.

After exiting the second SLM 1216b, the expanded laser beam 1254 may be directed to an f3 lens 1228. After exiting the f3 lens, the expanded laser beam 1254 may be directed to blocking element 1220. The blocking element 1220 may be immovable. The blocking element 1220 may be used to suppress illumination from a zero-order spot. After exiting the blocking element 1220, the expanded energy beam 1254 may be directed through an f4 lens 1238. The f4 lens 1238 may be a focusing lens. After exiting the f4 lens 1238, the expanded laser beam 1254 may be directed onto a second mirror 1214b and may be subsequently directed onto a third mirror 1214c. The third mirror 1214c may re-direct the expanded laser beam 1254 through a long pass dichroic mirror 1224. The first mirror 1214a, the second mirror 1214b, and/or the third mirror 1214c may be controlled with a computer system 1101. The computer system 1101 may turn the first mirror 1214a, the second mirror 1214b, and/or the third mirror 1214c “on” or “off” in order to re-direct the expanded laser beam 1254 as desired. The dichroic mirror may be a short pass dichroic mirror. The long pass dichroic mirror 1224 may reflect the expanded laser beam 1254 into the focusing objective 1232. In some instances, a beam combiner may be used to re-direct the expanded laser beam 1254 into the focusing objective 1232 instead of using the long pass dichroic mirror 1224. The long pass dichroic mirror 1224 may be controlled with a computer system 1101 to re-direct the expanded laser beam 1254 into the focusing objective 1232. The focusing objective 1232 may concentrate the expanded laser beam 1254 as it is projected into the printing chamber 1234. The printing chamber 1234 may be a media chamber 122. The printing chamber 1234 may comprise a cell-containing medium, a plurality of cells, cell constituents (e.g., organelles), and/or at least one polymer precursor.

The printing chamber 1234 may be mounted on a movable stage 1246. The movable stage 1246 may be an xy stage, a z stage, and/or an xyz stage. The movable stage 1246 may be manually positioned. The movable stage 1246 may be automatically positioned. The movable stage 1246 may be a motorized stage. The movable stage 1246 may be controlled by the computer system 1101. The computer system 1101 may control the movement of the movable stage 1246 in the x, y, and/or z directions. The computer system 1101 may automatically position the movable stage 1246 in a desired x, y, and/or z position. The computer system 1101 may position the movable stage 1246 in a desired x, y, and/or z position with a positional accuracy of at most about 3 μm. The computer system 1101 may position the movable stage 1246 in a desired x, y, and/or z position with a positional accuracy of at most about 2 μm. The computer system 1101 may position the movable stage 1246 in a desired x, y, and/or z position with a positional accuracy of at most about 1 μm. The computer system 1101 may automatically adjust the position of the movable stage 1246 prior or during three-dimensional printing. The computer system 1101 may comprise a piezo controller to provide computer-controlled z-axis (i.e., vertical direction) positioning and active location feedback. The computer system 1101 may comprise a joystick console to enable a user to control a position of the movable stage 1246. The joystick console may be a z-axis console and/or an x-axis and y-axis console. The movable stage 1246 may comprise a printing chamber holder. The printing chamber holder may be a bracket, a clip, and/or a recessed sample holder. The movable stage 1246 may comprise a multi-slide holder, a slide holder, and/or a petri dish holder. The movable stage 1246 may comprise a sensor to provide location feedback. The sensor may be a capacitive sensor. The sensor may be a piezoresistive sensor. The movable stage 1246 may comprise at least one actuator (e.g., piezoelectric actuator) that moves (or positions) the movable stage 1246.

A light-emitting diode (LED) collimator 1240 may be used as a source of collimated LED light 1256. The LED collimator 1240 may comprise a collimating lens and an LED emitter. The LED may be an inorganic LED, a high brightness LED, a quantum dot LED, or an organic LED. The LED may be a single color LED, a bi-color LED, or a tri-color LED. The LED may be a blue LED, an ultraviolet LED, a white LED, an infrared LED, a red LED, an orange LED, a yellow LED, a green LED, a violet LED, a pink LED, or a purple LED. The LED collimator 1240 may project a beam of collimated LED light 1256 through an f6 lens 1248. The f6 lens 1248 may be a focusing lens. Once the collimated LED light 1256 is transmitted through the f6 lens 1248, the collimated LED light 1156 may be directed into a light focusing objective 1236. The light focusing objective 1236 may focus the collimated LED light 1256 into the printing chamber 1234. The light focusing objective 1236 may focus the collimated LED light 1256 in the sample medium. The light focusing objective 1236 may focus the collimated LED light 1256 in the cell-containing medium. The collimated LED light 1256 may be transmitted through the printing chamber 1234 and into the focusing objective 1232. Once the collimated LED light 1256 exits the focusing objective 1232, the collimated LED light 1256 may be directed onto the long pass dichroic mirror 1224. The collimated LED light 1256 that is reflected off of the long pass dichroic mirror 1224 may be the sample emission 1226. The long pass dichroic mirror 1224 may re-direct the sample emission 1226 into an f5 lens 1244. The f5 lens may be a focusing lens. Once sample emission 1226 is transmitted through the f5 lens 1244, a detection system 1230 detects and/or collects the sample emission 1226 for imaging. The detection system 1230 may comprise at least one photomultiplier tube (PMT). The detection system 1230 may comprise at least one camera. The camera may be a complementary metal-oxide semiconductor (CMOS) camera, a scientific CMOS camera, a charge-coupled device (CCD) camera, or an electron-multiplying charge-coupled device (EM-CCD). The detection system 1230 may comprise at least one array-based detector.

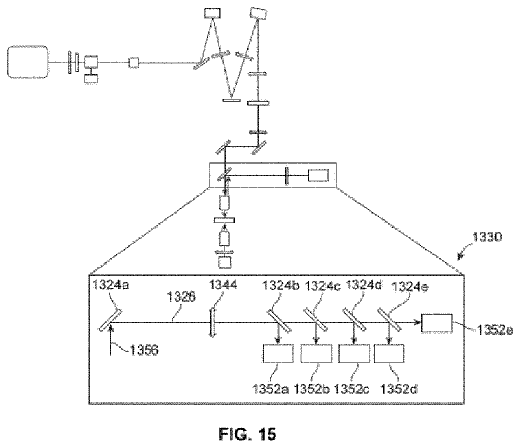

FIG. 15 illustrates a light detection system 1330. The light detection system 1330 may comprise a plurality of long pass dichroic mirrors arranged in series. The light detection system 1330 may comprise a plurality of long pass dichroic mirrors arranged in parallel. The light detection system 1330 may comprise a plurality of long pass dichroic mirrors arranged in series and parallel. As shown in FIGS. 44-46, the optical paths may comprise an LED collimator that projects a beam of collimated LED light 1356 onto the focusing objectives. Once the collimated LED light 1356 is reflected from the first long pass dichroic mirror 1324a, the collimated LED light 1356 may be converted to a sample emission 1326. The sample emission 1326 may be directed through an f5 lens 1344. The f5 lens 1344 may be a focusing lens. After the sample emission 1326 exits the f5 lens 1344, the sample emission 1326 may be directed to a series of long pass dichroic mirrors comprising a second long pass dichroic mirror 1324b, a third long pass dichroic mirror 1324c, a fourth long pass dichroic mirror 1324d, and a fifth long pass dichroic mirror 1324e, as shown in FIG. 15. The sample emission 1326 may be reflected off of the second long pass dichroic mirror 1324b and onto a first light detector 1352a. The sample emission 1326 may be reflected off of the third long pass dichroic mirror 1324c and onto a second light detector 1352b. The sample emission 1326 may be reflected off of the fourth long pass dichroic mirror 1324d and onto a third light detector 1352c. The sample emission 1326 may be reflected off of the fifth long pass dichroic mirror 1324e and onto a fourth light detector 1352d. The sample emission 1326 may be reflected off of the fifth long pass dichroic mirror 1324e and onto a fifth light detector 1352e. The light detector may be a photomultiplier tube (PMT). The light detector may be a camera. The light detector may be a complementary metal-oxide semiconductor (CMOS) camera, a scientific CMOS camera, a charge-coupled device (CCD) camera, or an electron-multiplying charge-coupled device (EM-CCD). The light detector may be an array-based detector. The light detection system 1330 may comprise a plurality of long pass dichroic mirrors that have progressively red-shifted cutoff wavelengths. In some instances, the second long pass dichroic mirror 1324b may have a cutoff wavelength of about 460 nm, the third long pass dichroic mirror 1324c may have a cutoff wavelength of about 500 nm, the fourth long pass dichroic mirror 1324d may have a cutoff wavelength of about 540 nm, the fifth long pass dichroic mirror 1324e may have a cutoff wavelength of about 570 nm. The light detection system 1330 may be controlled by the computer system 1101. The computer system 1101 may collect and/or process the signals obtained by the first light detector 1352a, the second light detector 1352b, the third light detector 1352c, and the fourth light detector 1352d. The computer system 1101 may provide control feedback to the three-dimensional printing system based on the light detector signals, of the light detection system 1330, which may be collected and/or processed by the computer system 1101. The computer system 1101 may have control feedback over any optical component and/or hardware of the optical paths described in FIGS. 44-46. The computer system 1101 may have control feedback over any optical component and/or hardware of the light detection system 1330 shown in FIG. 15. The computer system 1101 may control, for example, an SLM, a shutter, a movable stage, a mirror, a lens, a focusing objective, a beam expander, an LED collimator, a grating, and/or a blocking element in response to a signal from the light detection system 1330.

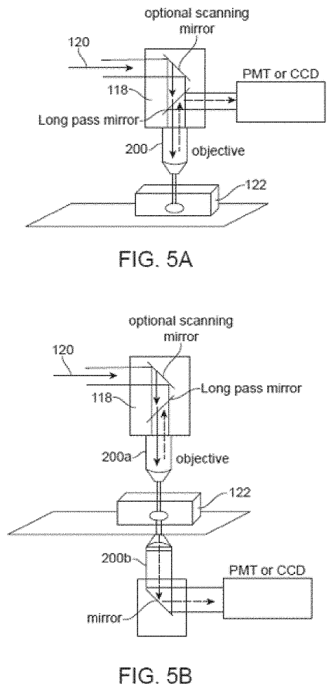

FIG. 5A illustrates an embodiment of the multi-photon tissue print head 118. The multi-photon print-head 118 may receive the multi-photon laser beam 120 (comprising one or more wavelengths) from the laser system 116 and may focus the beam 120 through the final optical path with is comprised of finishing optics that are comprised of an optional scan head, long pass mirror for use collection and recording of back-scatter light and a focusing objective 200, projecting the beam 120 into the media chamber 122. The light may be collected by the same objective as used to print, and then shunted via a long-pass mirror to the single or bank of PMTs, or a CCD camera.

In some designs, the optics may send the laser through a fiber optic cable for easier control of where the light is deposited in the tissue printing vessel.

The systems disclosed herein can utilize a range of focusing objectives, for example, with an increasingly lower magnification; the field of view may be increasingly larger. In some cases, the field of view may be the print area that the microscope is capable of, in a single projection area. In some cases, 5×, 10×, or 20× objectives may be employed. In some cases, objectives with high numerical apertures ranging between at least about 0.6 and about 1.2 or more may be employed. The systems disclosed herein may use an objective lens with a magnification ranging from e.g., about 1× to about 100×. The systems disclosed herein may use an objective lens with a magnification of about 1×. The systems disclosed herein may use an objective lens with a magnification of about 2×. The systems disclosed herein may use an objective lens with a magnification of about 3×. The systems disclosed herein may use an objective lens with a magnification of about 4×. The systems disclosed herein may use an objective lens with a magnification of about 10×. The systems disclosed herein may use an objective lens with a magnification of about 20×. The systems disclosed herein may use an objective lens with a magnification of about 40×. The systems disclosed herein may use an objective lens with a magnification of about 60×. The systems disclosed herein may use an objective lens with a magnification of about 100×.

To maintain structural fidelity of the printed tissues, a water-immersion objective lens may be ideal so as to substantially match the angle of incidence within the cell-containing liquid biogel media 126. A water-immersion objective lens corrected for refractive index changes may be used as printing takes place in liquid media which has a significantly different refractive index from air.

FIG. 5B illustrates a print head 118 comprising a first objective lens 200a and a second objective lens 200b. FIG. 5B illustrates inverted optics for imaging structures. In this embodiment, light may be collected by inverted optics and channeled to a CCD camera, a single PMT, as shown in FIG. 5B, or a bank of PMTs to create a multi-color image. In some embodiments, a second objective head may be inverted and images may be collected from the underside of the tissue and incident light read by PMTs with a series of long pass or band-pass mirrors.

In order for a multi-photon based printer to switch from a printing mode to an imaging mode, x, y raster scanning may be engaged and the DMD or SLM paths may be bypassed or the devices rendered in an off or inactive position, or removing them from the light path such that there is only a single laser line hitting the x, y scanning optics. DMD or SLM paths may also in some instances be used for imaging.

Switching to imaging mode may have several uses during the printing process: 1) imaging can be used to monitor collagen generation rates as collagen naturally produces an emission via second harmonic generation, which is a process when two-photon excitation is scanned across the structures, 2) the edges of printed tissues can be found using imaging mode facilitating the proper linking of blood vessels and other tissue structures along edges of projection spaces, 3) printed tissue structures can be validated for structural integrity and fidelity to the projected images in real-time, and 4) if cells that are temporarily labeled are used, they can be located within the printed tissues for process validation or monitoring.

It may be appreciated that the laser system 116 of the above embodiments may have a variety of points of software control including, but not limited to: The CAD images may be projected by programing changes that are hardwired to the SLM and/or DMD devices; If TAG lenses are used to create a Bessel beam, the current generated to induce the tunable acoustic gradient (TAG) in the TAG lens may be under the control of computer software; The mirrors that direct the laser excitation in the single beam incarnation and may act as an off/on switch for the multi-laser design may be controlled by computer software; The laser intensity via an attenuation wheel and tuning to different frequencies may be controlled by software input; Microscope stage movement may be under software control; Movement of microscope objective or associated fiber optics may be under software control; Edge finding, illumination, and control of the inverted objective by movement or on/off status may be under software control; any imaging or light path controls (mirrors, shutters, scanning optics, SLMs, DMD etc.) may be under control of software.

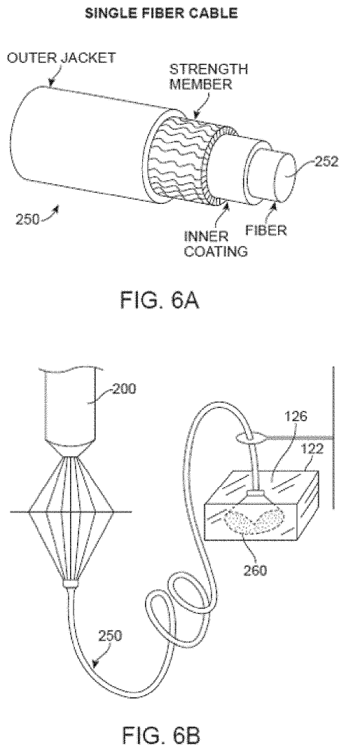

To accommodate rapid printing, the objective 200 may be equipped with a fiber optic cable. FIG. 6A illustrates an embodiment of a removable and attachable fiber optic cable accessory 250. In this embodiment, the accessory 250 may comprise a fiber optic cable 252 and a fitting (not shown in FIGS. 6A-6B) which is attachable to the multi-photon tissue printing print-head (not shown in 6A-6B). The fiber optic cable 252 can then be positioned within the media 126 of the media chamber 122, as illustrated in FIG. 6B. Thus, the multi-photon laser beam 120 may pass through the objective 200 and the fiber optic cable 252 to deliver the laser energy to the media 126, creating the desired complex tissue structure 260. To avoid moving the microscope objective during the printing process or the printing vessel that contains delicate tissue structures, the fiber optic cable itself may be moved if larger regions of tissue need to be printed. In some cases, the accessory 250 can be sterilized or replaced so that direct insertion into the media 126 does not compromise sterility or cross-contaminate printed cells.

Depending upon the power input into the fiber optic cable, multi-photon lasers may be capable of inducing irreversible damage to the core of the fiber optic cable. Thus, in some cases, induced wavelength chirping by group delayed dispersion (GDD) may be provided to minimize this potential damage, by effectively dispersing the photons to elongate the laser pulse. This may be used to either minimize damage to cells in the print media or to extend the life of fiber optic cables. In such instances, a GDD device may be provided in the laser system 116 after the SLM or DMD and before entry to the print-head optics 118.

In some cases, three-dimensional printing of the desired tissue may be carried out with a single objective 200 or an objective 200 with an attached fiber optic accessory 250, wherein the one to three different configurations, each associated with a distinct laser line and representing a distinct shape or portion of the tissue may be pulsed though the same objective 200. In such cases, a timed shutter system may be installed such that there is no or minimal interference between images being projected. Thus, laser multiplexing may be employed to allow generation of portions of the tissue structure simultaneously at multiple points while utilizing the same CAD model of the tissue structure. Likewise, the laser multiplexing may utilize different but contiguous CAD based tissue models, minimizing the movement needed for larger structure printing while decreasing overall print time further. For example, a vascular bed may have internal structures such as valves in the larger blood vessels that prevent venous back flow in normal circulation. These valve structures may be printed simultaneously with the blood vessel walls. In such a case, the scaffolding associated with the valve structure and/or blood vessel walls may be difficult to print separately.

The instantaneously formed three-dimensional structure may be repeated throughout the print space during one round of printing. In biological systems, small units may often be repeated throughout the structure. Therefore, repeated generation of a same structure in one print round may be useful for generating functional tissues. Additional, non-repetitive, fine featured structures and subsequent structures from the same cell-print material may be created that line-up with or link to the first structure printed.

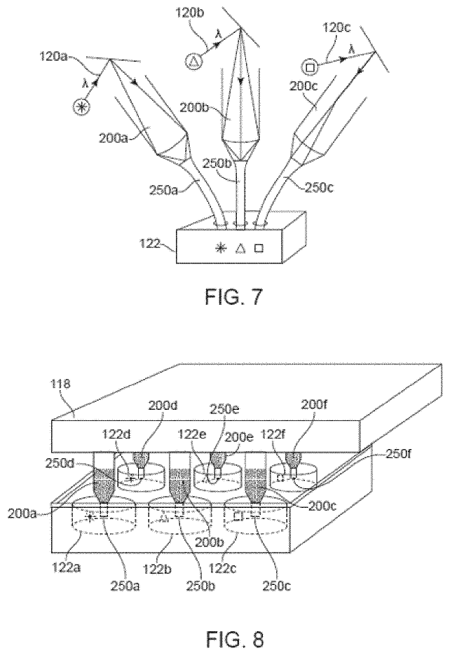

In some embodiments, the multi-photon tissue printing print-head 118 may include multiple printing “heads” or sources of multi-photon excitation via a first laser objective 200a, a second laser objective 200b, and a third laser objective 200c as illustrated in FIGS. 7-8. FIG. 7 illustrates an embodiment wherein the multi-photon tissue printing print-head 118 may include a first laser objective 200a, a second laser objective 200b, and a third laser objective 200c, wherein the first laser objective 200a may include a first fiber optic cable accessory 250a, the second laser objective 200b may include a second fiber optic cable accessory 250b, and the third laser objective 200c may include a third fiber optic cable accessory 250c. The first fiber optic cable accessory 250a, the second fiber optic cable accessory 250b, and the third fiber optic cable accessory 250c may be directed into a single media chamber 122. The media chamber 122 may have an open top or a sealed top with port access by each accessory fiber optic cable accessory (i.e., via the first fiber optic cable accessory 250a, the second fiber optic cable accessory 250b, and the third fiber optic cable accessory 250c). This arrangement may increase the speed of large, rapid tissue printing, while maintaining control over the final tissue structure. In some cases, the first fiber optic cable accessory 250a, the second fiber optic cable accessory 250b, and the third fiber optic cable accessory 250c may deliver a projection of the same tissue structure. In other cases, each the first fiber optic cable accessory 250a, the second fiber optic cable accessory 250b, and the third fiber optic cable accessory 250c may deliver a first laser beam projection 120a, a second laser beam projection 120b, and a third laser beam projection 120c, respectively, of a different tissue structure. Given the flexible arrangement of the multiple laser objectives and the ability of directing the fiber optic cables into the same area within the media chamber 122, the tissue structures may be simultaneously printed. The resulting tissue structures may be linked or not linked together. The print time of a given tissue structure may have an inverse relationship to the number of laser delivery elements with some consideration for the movement restrictions and considerations to be accounted for with each additional excitation source.

FIG. 8 illustrates an embodiment wherein the multi-photon tissue printing print-head 118 may include a first objective 200a, a second objective 200b, a third objective 200c, a fourth objective 200d, a fifth objective 200e, and a sixth objective 200f, wherein each objective may include a first fiber optic cable accessory 250a, a second fiber optic cable accessory 250b, a third fiber optic cable accessory 250c, a fourth fiber optic cable accessory 250d, a fifth fiber optic cable accessory 250e, and a sixth fiber optic cable accessory 250f, respectively, directed into a separate first media chamber 122a, a second media chamber 122b, a third media chamber 122c, a fourth media chamber 122d, a fifth media chamber 122e, and a sixth media chamber 122f, respectively. The plurality of media chambers may be a multi-well plate, wherein each well of the multi-well plate is a separate, individual media chamber. In some cases, the first fiber optic cable accessory 250a, the second fiber optic cable accessory 250b, the third fiber optic cable accessory 250c, the fourth fiber optic cable accessory 250d, the fifth fiber optic cable accessory 250e, and the sixth fiber optic cable accessory 250f may deliver at least one projection of the same tissue structure. This provides multiple copies of the tissue structure simultaneously. In other cases, the first fiber optic cable accessory 250a, the second fiber optic cable accessory 250b, the third fiber optic cable accessory 250c, the fourth fiber optic cable accessory 250d, the fifth fiber optic cable accessory 250e, and the sixth fiber optic cable accessory 250f may deliver a first multi-photon laser beam projection 120a, a second multi-photon laser beam projection 120b, and a third multi-photon laser beam projection 120c of a different tissue structure. In some cases, the print time may be greatly reduced due to the ability of producing multiple copies simultaneously.

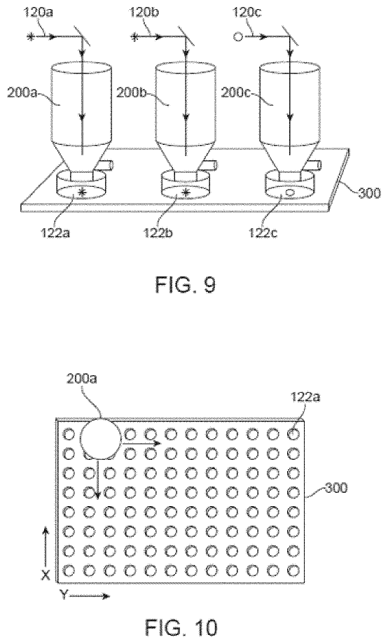

In some embodiments, the multi-photon tissue printing print-head 118 may include a serial array of objectives comprising a first objective 200a, a second objective 200b, and a third objective 200c, as illustrated in FIG. 9. In this embodiment, each objective may be aligned with a separate media chamber. For example, the first objective 200a may be aligned with a first media chamber 122a, the second objective 200b may be aligned with a second media chamber 122b, the third objective 200c may be aligned with a third media chamber 122c. In some instances, the multiple media chambers may be wells of a multi-well plate 300. In some embodiments, the first objective 200a, the second objective 200b, and the third objective 200c may deliver projection of the same tissue structure. In other cases, the laser beam projections may differ per well. The first objective 200a, the second objective 200b (not shown in FIG. 10), and the third objective 200c (not shown in FIG. 10) may be programmed to move over the multi-well plate 300 in the x and y directions, as illustrated in FIG. 10, to deliver the laser beam projections into each well. Alternatively, it may be appreciated that the objectives may remain stationary while the multi-well plate 300 moves in the x and y directions. Thus, for example, a serial array having three objectives can print tissue in a six well plate in two steps: three tissue structures simultaneously and then three more tissue structures simultaneously. It may be appreciated that plates having any number of wells may be used including, but not limited to at least about 96 wells to about 394 wells, or more. The multi-well plate 300 may comprise at least a first media chamber 122a. The multi-well plate 300 may comprise at least 1 well. The multi-well plate 300 may comprise at least 4 wells. The multi-well plate 300 may comprise at least 6 wells. The multi-well plate 300 may comprise at least 8 wells. The multi-well plate 300 may comprise at least 12 well. The multi-well plate 300 may comprise at least 16 wells. The multi-well plate 300 may comprise at least 24 wells. The multi-well plate 300 may comprise at least 48 wells. The multi-well plate 300 may comprise at least 96 wells. The multi-well plate 300 may comprise at least 384 wells. The multi-well plate 300 may comprise at least 1536 wells.

It may be appreciated that in the embodiments described herein, the microscope stage may be able to move, the microscope head may be able to move, and/or an associated fiber optic cable attached to the printing objective may be able to move in order to print larger spaces.

### Methods of Printing Three-Dimensional Matrices

The present disclosure provides methods and systems of printing and using a three-dimensional cell-containing matrix. In an aspect, a method of using a three-dimensional (3D) cell-containing matrix comprises: providing a media chamber comprising a medium comprising (i) a plurality of cells and (ii) one or more polymer precursors. Next, the method may comprise directing at least one energy beam to the medium in the media chamber along at least one energy beam path that is patterned into a three-dimensional (3D) projection in accordance with computer instructions for printing the 3D cell-containing medical device in computer memory, to form at least a portion of the 3D cell-containing matrix comprising (i) at least a subset of the plurality of cells, and (ii) a polymer formed from the one or more polymer precursors. Next, the method may comprise positioning the 3D cell-containing matrix in a subject.

In another aspect, a method of using a three-dimensional (3D) cell-containing matrix, comprises (i) printing the 3D cell-containing matrix comprising a plurality of cells, and (ii) positioning the 3D cell-containing matrix in a subject.

In another aspect, a method for using a three-dimensional (3D) cell-containing matrix, comprises providing a media chamber comprising a first medium. The first medium may comprise a first plurality of cells and a first polymeric precursor. Next, the method may comprise directing at least one energy beam to the first medium in the media chamber along at least one energy beam path in accordance with computer instructions for printing the 3D cell-containing matrix in computer memory, to subject at least a portion of the first medium in the media chamber to form a first portion of the 3D cell-containing matrix. Next, the method may comprise providing a second medium in the media chamber. The second medium may comprise a second plurality of cells and a second polymeric precursor. The second plurality of cells may be of a different type than the first plurality of cells. Next, the method may comprise directing at least one energy beam to the second medium in the media chamber along at least one energy beam path in accordance with the computer instructions, to subject at least a portion of the second medium in the media chamber to form a second portion of the 3D cell-containing matrix. Next, the method may comprise positioning the first and second portions of the 3D cell-containing matrix in a subject.

In another aspect, a method of using a three-dimensional (3D) cell-containing matrix, comprises (i) printing the 3D cell-containing matrix comprising a first plurality of cells and a second plurality of cells. The first plurality of cells may be different from the second plurality of cells. Next, the method may comprise (ii) positioning the 3D cell-containing matrix in a subject.

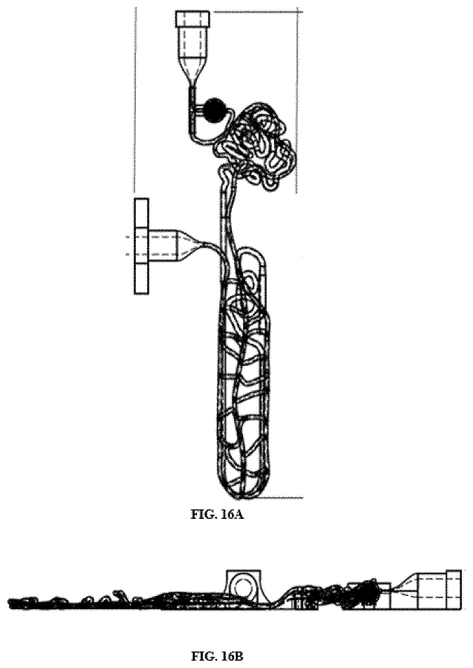

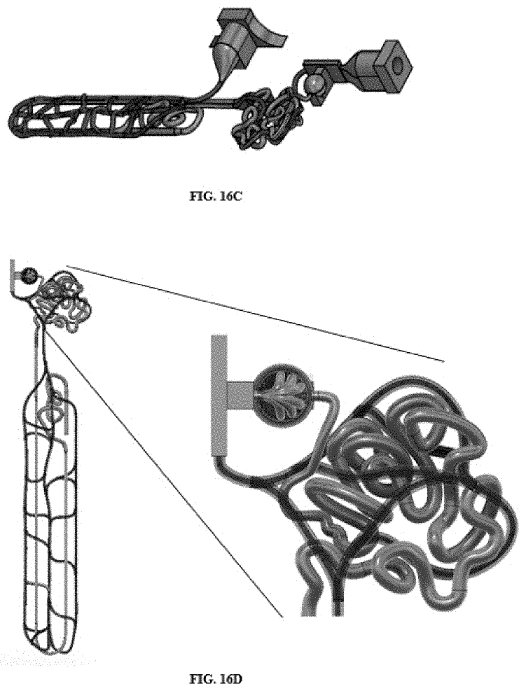

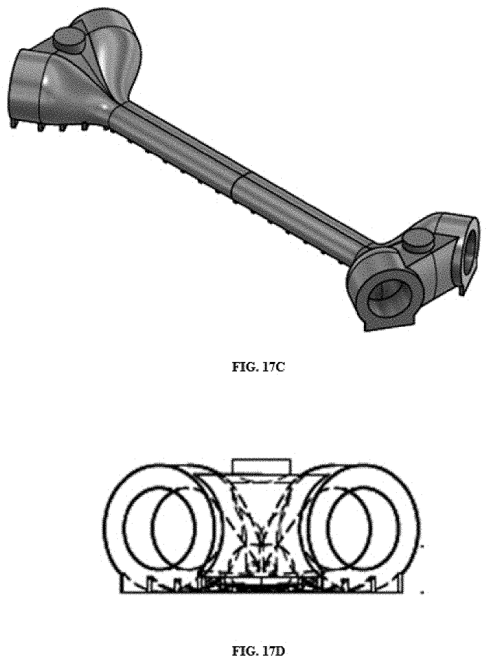

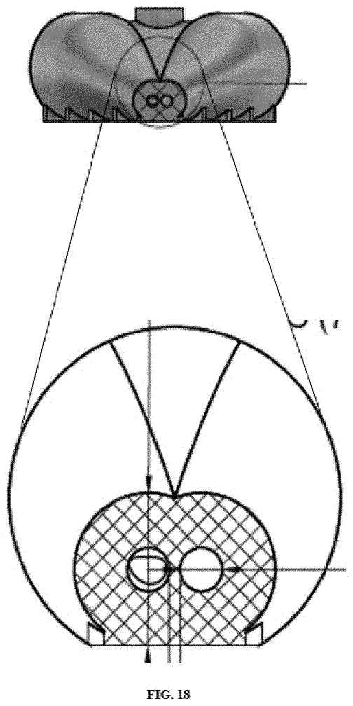

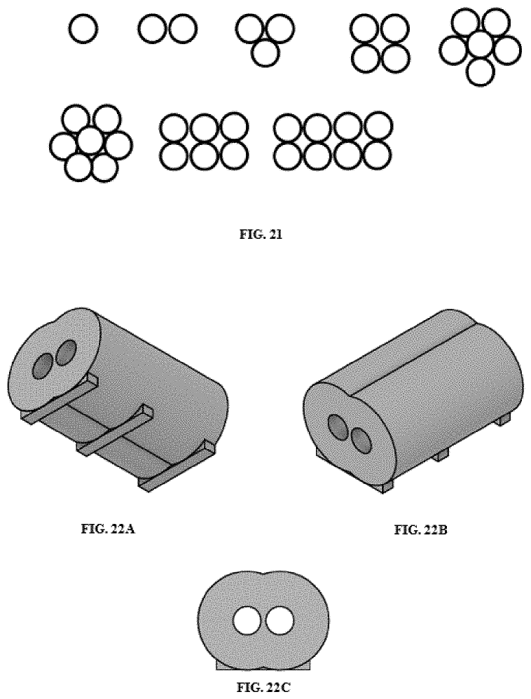

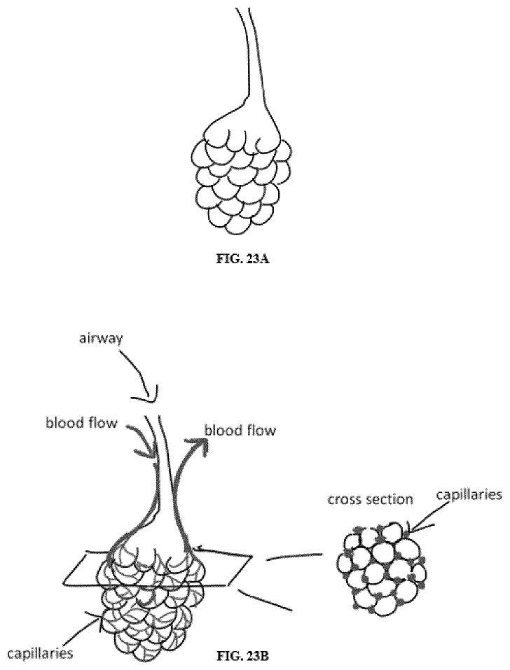

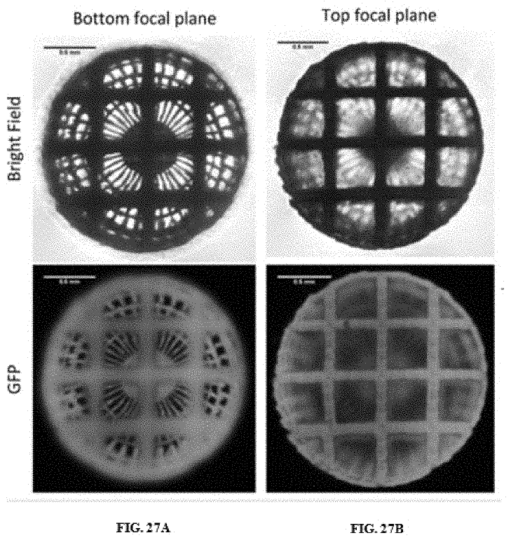

The 3D cell-containing matrix may be an alveolar structure, as shown in FIGS. 23-25. The 3D cell-containing matrix may be a nephron structure, as shown in FIGS. 16-22. The 3D cell-containing matrix may be a capillary structure, as shown in FIG. 28. For example, the 3D cell-containing matrix may be a capillary bed, a vascular bed, a group of microscopic blood vessels. In some examples, the 3D cell-containing matrix may be a group of tubes with diameters and lengths in the order of microns. In some examples, the 3D cell-containing matrix may be a group of tubes with diameters and lengths in the order of microns. The diameter of a 3D cell-containing matrix may range between at least about 1 micron (μm) to about 10 μm.

The present disclosure provides methods and systems of printing and using a three-dimensional matrix that does not contain cells. The three-dimensional matrix that does not contain cells may have the same structure, dimensions, and physical characteristics as the 3D cell-containing matrices described elsewhere herein.

In another aspect, the 3D cell-containing matrix may form a suture, stent, staple, clip, strand, patch, graft, sheet, tube, pin, or screws. The graft may be selected from the list consisting of skin implant, uterine lining, neural tissue implant, bladder wall, intestinal tissue, esophageal lining, stomach lining, hair follicle embed skin, and retina tissue.

The plurality of cells may be from a subject. The method plurality of cells may be selected from the list consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thin segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes.

The plurality of cells may further be selected from naïve B cells or other immature B cells, memory B cells, plasma B cells, helper T cells and subsets of the same, effector T cells and subsets of the same CD+8 T cells, CD4+ T cells, regulatory T cells, natural killer T cells, naïve T cells or other immature T cells, dendritic cells and subsets of the same, follicular dendritic cells, Langerhans dendritic cells, dermally-derived dendritic cells, dendritic cell precursors, monocyte-derived dendritic cells, monocytes and subsets of the same macrophages and subsets of the same, leukocytes and subsets of the same. The B cells may be selected from the list consisting of naïve B cells, mature B cells, plasma B cells, B1 B cells and B2 B cells. The T cells may be selected from the list consisting of CD8+ and CD4+.

The 3D cell-containing matrix may be from about 1 micrometer (μm) to about 10 centimeters (cm). The 3D cell-containing matrix may be from at least about 5 μm to about 10 cm or more. The 3D cell-containing matrix may be from at least about 10 μm to about 10 cm or more. The 3D cell-containing matrix may be from at least about 100 μm to about 10 cm or more. The 3D cell-containing matrix may be from at least about 500 μm to about 10 cm or more. The 3D cell-containing matrix may be from at least about 1000 μm to about 10 cm or more. The 3D cell-containing matrix may be from at least about 1 cm to about 10 cm or more. The 3D cell-containing matrix may be from about at least 5 to about 10 cm or more.

The 3D cell-containing matrix may be about 1 μm to about 1,000 μm. The 3D cell-containing matrix may be at least about 1 μm. The 3D cell-containing matrix may be at most about 1,000 μm. The 3D cell-containing matrix may be about 1 μm to about 5 μm, about 1 μm to about 10 μm, about 1 μm to about 100 μm, about 1 μm to about 1,000 μm, about 5 μm to about 10 μm, about 5 μm to about 100 μm, about 5 μm to about 1,000 μm, about 10 μm to about 100 μm, about 10 μm to about 1,000 μm, or about 100 μm to about 1,000 μm. The 3D cell-containing matrix may be about 1 μm, about 5 μm, about 10 μm, about 100 μm, or about 1,000 μm.

The 3D cell-containing matrix may be about 0.5 cm to about 10 cm. The 3D cell-containing matrix may be at least about 0.5 cm. The 3D cell-containing matrix may be at most about 10 cm. The 3D cell-containing matrix may be about 0.5 cm to about 1 cm, about 0.5 cm to about 2 cm, about 0.5 cm to about 3 cm, about 0.5 cm to about 4 cm, about 0.5 cm to about 5 cm, about 0.5 cm to about 6 cm, about 0.5 cm to about 7 cm, about 0.5 cm to about 8 cm, about 0.5 cm to about 9 cm, about 0.5 cm to about 10 cm, about 1 cm to about 2 cm, about 1 cm to about 3 cm, about 1 cm to about 4 cm, about 1 cm to about 5 cm, about 1 cm to about 6 cm, about 1 cm to about 7 cm, about 1 cm to about 8 cm, about 1 cm to about 9 cm, about 1 cm to about 10 cm, about 2 cm to about 3 cm, about 2 cm to about 4 cm, about 2 cm to about 5 cm, about 2 cm to about 6 cm, about 2 cm to about 7 cm, about 2 cm to about 8 cm, about 2 cm to about 9 cm, about 2 cm to about 10 cm, about 3 cm to about 4 cm, about 3 cm to about 5 cm, about 3 cm to about 6 cm, about 3 cm to about 7 cm, about 3 cm to about 8 cm, about 3 cm to about 9 cm, about 3 cm to about 10 cm, about 4 cm to about 5 cm, about 4 cm to about 6 cm, about 4 cm to about 7 cm, about 4 cm to about 8 cm, about 4 cm to about 9 cm, about 4 cm to about 10 cm, about 5 cm to about 6 cm, about 5 cm to about 7 cm, about 5 cm to about 8 cm, about 5 cm to about 9 cm, about 5 cm to about 10 cm, about 6 cm to about 7 cm, about 6 cm to about 8 cm, about 6 cm to about 9 cm, about 6 cm to about 10 cm, about 7 cm to about 8 cm, about 7 cm to about 9 cm, about 7 cm to about 10 cm, about 8 cm to about 9 cm, about 8 cm to about 10 cm, or about 9 cm to about 10 cm. The 3D cell-containing matrix may be about 0.5 cm, about 1 cm, about 2 cm, about 3 cm, about 4 cm, about 5 cm, about 6 cm, about 7 cm, about 8 cm, about 9 cm, or about 10 cm.

The 3D cell-containing matrix may be at least about 1 μm or more. The 3D cell-containing matrix may be at least about 5 μm or more. The 3D cell-containing matrix may be at least about 10 μm or more. The 3D cell-containing matrix may be at least about 50 μm or more. The 3D cell-containing matrix may be at least about 100 μm or more. The 3D cell-containing matrix may be at least about 1000 μm or more. The 3D cell-containing matrix may be at least about 0.5 cm or more. The 3D cell-containing matrix may be at least about 1 cm or more. The 3D cell-containing matrix may be at least about 5 cm or more. The 3D cell-containing matrix may be at least about 10 cm or more.

The media chamber comprising a medium of a plurality of cells and one or more polymer precursors may comprise a volume of at least about 0.1 cubic nanometers. The media chamber may comprise a volume of at least about 0.2, 0.3, 0.4, 0.5, 0.6, 0.7, 0.8, 0.9, 1, 1.5, 2, 2.5, 3, 4, 5, 6, 7, 8, 9, 10, 20, 30, 40, 50, or more cubic nanometers. The media chamber may comprise a volume of at most about 1×10²⁰, 1×10²⁰, 1×10¹⁹, 1×10¹⁸, 1×10¹⁷, 1×10¹⁶, 1×10¹⁵, 1×10¹⁴, 1×10¹³, 1×10¹², 1×10¹¹, 1×10⁹, 1×10⁸, 1×10⁷, 1×10⁶, 1×10⁵, 1×10⁴, 1,000, 100, 90, 80, 70, 60, or less cubic nanometers.

The 3D cell-containing matrix may comprise an agent to promote growth of vasculature or nerves. The agent may be selected from the group consisting of growth factors, cytokines, chemokines, antibiotics, anticoagulants, anti-inflammatory agents, opioid pain-relieving agents, non-opioid pain-relieving agents, immune-suppressing agents, immune-inducing agents, monoclonal antibodies and stem cell proliferating agents.

Another aspect of the present disclosure provides a system for producing one or more 3D cell-containing matrices, comprising a media chamber configured to contain a first medium comprising a first plurality of cells and a first plurality of polymer precursors. The system may comprise at least one energy source configured to direct at least one energy beam to the media chamber. The system may comprise one or more computer processors operatively coupled to the at least one energy source. The one or more computer processors may be individually or collectively programmed to receive computer instructions for printing a three-dimensional (3D) cell-containing matrices from computer memory. The one or more computer processors may be individually or collectively programmed to direct the at least one energy source to direct the at least one energy beam to the first medium in the media chamber along at least one energy beam path in accordance with the computer instruction, to subject at least a portion of the first polymer precursors to form at least a portion of the 3D cell-containing matrices. The one or more computer processors may be individually or collectively programmed to direct the at least one energy source to direct the at least one energy beam to a second medium in the media chamber along at least one energy beam path in accordance with the computer instructions, to subject at least a portion of the second medium in the media chamber to form at least a second portion of the 3D cell-containing matrices. The second medium may comprise a second plurality of cells and a second plurality of polymeric precursors. The second plurality of cells may be of a different type than the first plurality of cell. The one or more computer processors may be individually or collectively programmed to subject the first and second portions of the 3D cell-containing matrices to conditions sufficient to stimulate production of the one or more immunological proteins. The one or more computer processors may be individually or collectively further programmed to extract the one or more immunological proteins from the first and second portions of the 3D cell-containing matrices.

Materials that may be used to print 3D cell-containing matrices or devices include degradable polymers, non-degradable polymers, biocompatible polymers, extracellular matrix components, bioabsorbable polymers, hydrogels, or any combination thereof. Non-limiting examples of bioasborbable polymers include polyesters, polyamino acids, polyanhydrides, polyorthoesters, polyurethanes, and polycarbonates. Non-limiting examples of biocompatible polymers include collagen, hyaluronic acid and other glycosaminoglycans, poly-dl-lactic-co-glycolic acid (PLGA), poly-1-lactic acid (PLLA), polyglycolic acid (PGA), alginate, gelatin, agar, or a combination thereof. The biocompatible polymer may comprise an extracellular matrix component. Non-limiting examples of extracellular matrix components may include proteoglycans such as heparan sulfate, chondroitin sulfate, and keratan sulfate, non-proteoglycan polysaccharide such as hyaluronic acid, collagen, and elastin, fibronectin, laminin, nidogen, or any combination thereof. These extracellular matrix components may be functionalized with acrylate, diacrylate, methacrylate, cinnamoyl, coumarin, thymine, or other side-group or chemically reactive moiety to facilitate cross-linking induced directly by multi-photon excitation or by multi-photon excitation of one or more chemical doping agents. In some cases, photopolymerizable macromers and/or photopolymerizable monomers may be used in conjunction with the extracellular matrix components to create cell-containing structures. Non-limiting examples of photopolymerizable macromers may include polyethylene glycol (PEG) acrylate derivatives, PEG methacrylate derivatives, and polyvinyl alcohol (PVA) derivatives. In some instances, collagen used to create cell containing structure may be fibrillar collagen such as type I, II, III, V, and XI collagen, facit collagen such as type IX, XII, and XIV collagen, short chain collagen such as type VIII and X collagen, basement membrane collagen such as type IV collagen, type VI collagen, type VII collagen, type XIII collagen, or any combination thereof.

The biocompatible polymer may comprise other polymerizable monomers that are synthesized and not native to mammalian tissues, comprising a hybrid of biologic and synthetic materials. The biocompatible polymer may comprise a photoinitiator. Non-limiting examples of photoinitiators may include azobisisobutyronitrile (AIBN), benzoin derivatives, benziketals, hydroxyalkylphenones, acetophenone derivatives, trimethylolpropane triacrylate (TPT), acryloyl chloride, benzoyl peroxide, camphorquinone, benzophenone, thioxanthones, and 2-hydroxy-1-[4-(hydroxyethoxy)phenyl]-2-methyl-1-propanone. Hydroxyalkylphenones may include 4-(2-hydroxyethylethoxy)-phenyl-(2-hydroxy-2-methyl propyl) ketone (Irgacure® 295), 1-hidroxycyclohexyl-1-phenyl ketone (Irgacure® 184) and 2,2-dimethoxy-2-phenylacetophenone (Irgacure® 651). Acetophenone derivatives may include 2,2-dimethoxy-2-phenylacetophenone (DMPA). Thioxanthones may include isopropyl thioxanthone.

Once in place, the device may bring two pieces of tissue together, the cells may migrate within or out of the device, interact with other cells locally to promote healing and tissue remodeling around or within the cell containing bio-resorbable device. The cell-containing, bio-resorbable medical devices may be sutures of any length or width, staples, stents of any length or width, clips which may be locking or compressible, patches and grafts of arbitrary shapes and sizes, and/or similar structures intended to be used in a living subject. Single or multi-layered patches and grafts of arbitrary shape and size can be created out of multiple different cells types to promote tissue development, augment tissue function, and/or healing. Grafts may include but are not limited to: skin implant, uterine lining, neural tissue implant, bladder wall, intestinal tissue, esophageal lining, stomach lining, hair follicle embedded skin, retina tissue, or any combination thereof.

The holographically printed grafts and/or patches may comprise a variety of shapes, such as but not limited to oblong, rectangular, oval, any other polygonal shape, or any amorphous shape required to repair or reinforce the site of injury or disease.

To enhance the structural integrity of some devices three-dimensionally printed materials may be thicker or denser and may or may not contain cells at all sites. These cells when printed are trapped in any size aperture to keep cells in place, or allow them to move from the site in which they were originally printed and interact with other cells within their own layer, cells in subsequently or previously printed layers, or with cells in the native tissue that they are eventually implanted in. Cells encapsulated, embedded, trapped, or contained within a mesh net, lattice, matrix, framework of any aperture size or density that allow cells to move through the apertures during the developmental process or be trapped in place. This makes up the base components of a larger structural architecture.

Three-dimensional lithography may be used to generate functional partial organs or organoids that may serve an augmenting or independent physiologic function not necessarily dependent upon site of implantation.

Non-limiting examples of tissues for augmentation or replacement of function include kidney or generative models of kidney tissues, lung tissue or partial or full lung lobes and generative models therein, neural tissues, pancreatic tissues, insulin producing beta islets and associated tissues, thyroid tissues, splenic tissues, liver tissues, tissues of the intestinal tract. All tissues listed necessarily include all structural components and accessory cells necessary to impart functional capabilities, included but not limited to, vasculature large and small as well as lymphatic drainage systems and all associated hollow structures, and nerve and, or immune cells necessary to impart functional capabilities.

In some embodiments, a printed kidney generative model is generated by the methods disclosed herein. The basic structural component of a kidney, including but not limited to: urine collecting ducts, vascularized and dense tissue surrounding urine collecting ducts, and kidney capsule may be separated into separate computer-aided design (CAD) files and printed sequentially, but in any order necessary, with automated computer control programs 1101. Printing may be achieved by signaling computer files to the laser printing system 110, and the structure that mimics the CAD files may be deposited sequentially, but in an order necessary, into the biogel and media chamber 122.

Three-dimensionally printed structures for implantation may be on the order of 1 micron to tens of centimeters or greater in volume. The surface area of complex tissue structures such as the lung take up several square meters and thus the external size of a large printed organ will be necessarily different from the surface area of the functional units. Therefore, the methods and systems provided herein may be designed to cover all structural components within the physiologic range of functional sizes and surface to volume ratios.

Laser-based holography may be used to near-instantaneously polymerize biomatrix materials in set patterns projected from computer aided design (CAD) files by a spatial light modulator or digital mirror device. Multiple print steps and positions may be required to build a full generative model.

Cells may be in any state of genetic or phenotypic differentiation, including undifferentiated, partially differentiated, fully differentiated. Examples of differentiation states include, but are not limited to pluripotent stem cells, totipotent stem cells. Cells may be autologous cells, sourced from a matched donor, cord blood, or an established cell line. Multiple cell types at the same and/or different differentiation state may be used within a single print layer and/or multiple iterative print layers. Cells may be genetically manipulated prior to, during, and, or after the printing process via optical switch technology, clustered regularly interspaced short palindromic repeats (CRISPR) technology, introduction of virus, or other approaches for genetic manipulation. Genetic manipulation is not limited to nuclear DNA and may include mitochondrial DNA or free-floating plasmids or viral DNA not intended for incorporation into nuclear DNA.

Printed structures may comprise cells at high density or variable, including lop-sided cell densities or controlled densities of cells to promote cellular expansion or niche development in specific sites of the device. High or low cell density may be used depending on tissue product needs. Low cell density may be as low as 10,000 cells per cubic centimeter of printed material and as high as 1 billion cells per cubic centimeter of printed materials. Cells may be of one type or mixed and printing may be performed in multiple layers.

Bioprinting materials may contain agents intended to promote growth of vasculature, including microvasculature, and nerves into the printed structure or into the surrounding native architecture. Such agents include but are not limited to: growth factors, cytokines, chemokines, antibiotics, anticoagulants, anti-inflammatory agents, opioid or non-opioid pain-relieving agents, immune-suppressing agents, immune-inducing agents, monoclonal antibodies, and/or stem cell proliferating agents.

The present disclosure provides methods and systems for using a three-dimensional (3D) matrix. In an aspect, a method for using a three-dimensional (3D) matrix comprises providing a media chamber comprising a medium comprising: a) a plurality of cells and a first polymeric precursor. Next, the method comprises b) directing at least one energy beam to the medium in the media chamber along at least one energy beam path that is patterned into a three-dimensional (3D) projection in accordance with computer instructions for printing a 3D cell-containing matrix in computer memory. This may form at least a first portion of the 3D cell-containing matrix comprising. Next, the method may comprise providing a second medium in the media chamber, wherein the second medium comprises a second plurality of cells and a second polymeric precursor, wherein the second plurality of cells is of a different type than the first plurality of cells. Next, the method may comprise d) directing at least one energy beam to the second medium in the media chamber along at least one energy beam path in accordance with the computer instructions, to subject at least a portion of the second medium in the media chamber to form a second portion of the 3D cell-containing matrix. Next, the method may comprise e) positioning the first and second portions of the 3D cell-containing matrix in a subject.

The present disclosure provides a method of using a three-dimensional (3D) cell-containing matrix. In an aspect, the method comprises (i) printing the 3D cell-containing matrix comprising a first plurality of cells and a second plurality of cells, wherein the first plurality of cells is different from the second plurality of cells, and (ii) positioning the 3D cell-containing matrix in a subject.

Another aspect of the present disclosure provides a system for producing one or more 3D cell-containing matrix, comprising a media chamber configured to contain a medium comprising a plurality of cells and one or more polymer precursors. The system may comprise at least one energy source configured to direct at least one energy beam to the media chamber. The system may comprise one or more computer processors operatively coupled to the at least one energy source. The one or more computer processors may be individually or collectively programmed to receive computer instructions for printing a three-dimensional (3D) cell-containing matrix from computer memory. The one or more computer processors may be individually or collectively programmed to direct the at least one energy source to direct the at least one energy beam to the medium in the media chamber along at least one energy beam path in accordance with the computer instructions, to subject at least a portion of the polymer precursors to form at least a portion of the 3D cell-containing matrix. The one or more computer processors may be individually or collectively programmed to subject the at least portion of the 3D cell-containing matrix to conditions sufficient to stimulate production of the one or more immunological proteins. The one or more computer processors may be individually or collectively further programmed to extract one or more immunological proteins from the at least portion of the 3D cell-containing matrix.

Another aspect of the present disclosure provides a system for producing one or more 3D cell-containing matrices, comprising: a media chamber configured to contain a first medium comprising a first plurality of cells and a first plurality of polymer precursors. The system may comprise at least one energy source configured to direct at least one energy beam to the media chamber. The one or more computer processors operatively coupled to the at least one energy source, wherein the one or more computer processors are individually or collectively programmed to receive computer instructions for printing a three-dimensional (3D) cell-containing matrix from computer memory. The one or more computer processors are individually or collectively programmed direct the at least one energy source to direct the at least one energy beam to the first medium in the media chamber along at least one energy beam path in accordance with the computer instruction, to subject at least a portion of the first polymer precursors to form at least a portion of the 3D cell-containing matrix. The one or more processors may be individually or collectively programmed to direct the at least one energy source to direct the at least one energy beam to a second medium in the media chamber along at least one energy beam path in accordance with the computer instructions, to subject at least a portion of the second medium in the media chamber to form at least a second portion of the 3D cell-containing matrix, wherein the second medium comprises a second plurality of cells and a second plurality of polymeric precursors, wherein the second plurality of cells is of a different type than said first plurality of cell.

In an aspect, the present disclosure provides a method of printing an organ and/or an organoid. The method may comprise polymerization of a photopolymerizable material by a laser light source. The organ and/or the organoid may be two-dimensional or three-dimensional. The organ and/or the organoid may be a lymph node. The organoid may be an islet of Langerhans. The organoid may be a hair follicle. The organ and/or the organoid may be a tumor and/or a tumor spheroid. The organoid may be a neural bundle and support cells such as, but not limited to Schwann cells and glial cells including satellite cells, olfactory ensheathing cells, enteric glia, oligodendroglia, astroglia, and/or microglia. The organoid may be a nephron. The organoid may be an alveolus. The organoid may be a liver organoid. The organoid may be an intestinal crypt. The organ and/or the organoid may be a primary lymphoid organ, a secondary lymphoid organ such as a spleen, a liver, a pancreas, a gallbladder, an appendix, a brain, a small intestine, a large intestine, a heart, a lung, a bladder, a kidney, a bone, a cochlea, an ovary, a thymus, a trachea, a cornea, a heart valve, skin, a ligament, a tendon, a muscle, a thyroid gland, a nerve, and/or a blood vessel.

Organization of an organ or organoid through the printing process, disclosed herein, may require or be implemented by the sequential deposition of at least about 1, 10, 50, 100, 200, 300, 500, 600, 700, 800, 900, 1000, 10000, 100000, 1000000 or more layers of cells. Organization of a lymphoid organ through the printing process may require or be implemented by the sequential deposition of between 1 and 100 layers of cells. The size of a layer of cells may be tissue dependent. The size of a layer of cells may comprise a larger three-dimensional structure that may be one layer of cells or may comprise multiple layers of cells. The layer of cells may comprise about at least 10, 10², 10³, 10⁴, 10⁵, 10⁶, 10⁷, 10⁸, 10⁹, 10¹⁰, or more cells. Where precise placement of each cell type relative to the other is desired, cells should be printed in sequential steps with a wash step in between to remove the previously used media. Alternately, two or more cell types of different sizes may be printed simultaneously using two photopolymerizable materials of different polymerization wavelength and pore size, such that the larger cell type may become encapsulated in the pore of larger size and the smaller cell type may become encapsulated in the pore of smaller size. Cells are encapsulated in pores in accordance with the size of their nucleus, as the cytoskeleton is able to remodel based on the available space.

The laser light source may use high-energy green, blue, white, or lower frequencies of ultraviolet light to induce polymerization of the photopolymerizable material, or a high-resolution multi-photon light source of any wavelength may be used. The high-resolution, non-toxic multi-photon projection technology is uniquely suited to print detailed germinal centers that allow for the development of light and dark zones that recapitulate natural B cell affinity maturation. This method may be used in combination with microfluidic manipulation of vasculature, whether lymphatic or circulatory, to create functional collagen-based organs and/or organoids, such as lymph node organoids. Nontoxic wavelengths of visible and ultraviolet light may alternatively be used to print cell-containing structures or biogels to be seeded with cells.

The present disclosure encompasses the printing of organs or organoids by two- or three-dimensional projection of a laser beam 1002 from an energy source 1000 (i.e., a laser, especially a high-resolution multi-photon laser beam but also including other possible light sources). The laser beam 1002 is intended to induce polymerization of a cell-containing media 126 in a predefined pattern to produce a final product that resembles in structure or function native, especially human organs or organoids. Human organs and organoids are herein defined as small, fully functional, immune cell-containing structures that are capable of mounting and carrying out a functional and complete biological response, e.g., gaseous exchange or filtration of a fluid such as, but not limited to a biological fluid.

Where cells are printed within a network, the network may be arranged in a reticular, amorphous, or organized net. An organized net is any net with a repeated geometric or other pattern, including hexagonal, square/rectangular, rhomboid, circular, semi-circular, spherical, semi-spherical, or any combination of shapes therein. A reticular or amorphous net is created without significant regard for geometric pattern, with the primary purpose of being created rapidly and being capable of encapsulating and containing cells. Additionally, some nets may appear amorphous to the untrained observer but, in fact, have a specific shape or design designed to facilitate cellular interactions or movement between or within cellular niches.

Native architecture may be obtained from imaging data and rendered into two- or three-dimensional images with defined edges and/or grey areas, which are edges that are not precisely defined, but fall somewhere within a designated range, for projection into a polymerizable hydrogel.

Multiple organoid units may be printed within a single structure to produce larger organs, up to and including a fully sized organ. Multiple organoids units may be printed within a single structure to produce larger organs, up to and including a fully sized nephron or alveolus. The limiting factor for size is vascularization, which is essential for tissues larger than 200 micron in width due to the diffusion limits of most gases and nutrients. The completed organ or organoid may be between 50 and 200 microns thick without vascularization. If vascularized, the tissue may be 50 microns to 10 cm thick, may be of any shape or size, and may contain both circulatory and lymphatic vasculature. Vasculature may include valves and/or sphincters. In some embodiments, vasculature may be achieved by printing endothelial cells or precursors thereof within a net intended to closely resemble native microvasculature, the structure of which is obtained from high-resolution imaging data. Capillary beds may branch from larger arterioles and arteries and branch into venules and veins in accordance with the relevant anatomy.

In an aspect, the present disclosure provides a method of producing a population of 3D matrices, e.g., a nephron structure, an alveolar structure, and/or a capillary structure. The method may comprise providing a medium. The medium may comprise a plurality of cells and one or more polymer precursors. The polymer precursors may be biogel precursors. The method may comprise depositing at least one layer of the medium onto a substrate. The substrate may be a media chamber. The substrate may be a tissue culture plate or well. The substrate may be a microfluidic chamber. The substrate may be a microfluidic chip. The substrate may be a polymeric scaffold.

The method may comprise subjecting the at least one layer of the medium to an energy source to form at least a portion of the 3D matrix and a biogel, formed from the one or more polymer precursors. In some examples, the 3D matrix may comprise at least a subset of the plurality of cells. Alternatively, in yet another example, the 3D matrix may not comprise a plurality of cells. The method may comprise a layer-by-layer deposition of the medium patterned according to a three-dimensional (3D) projection. The 3D projection may be in accordance with computer instructions for printing the 3D matrix in computer memory. The layer-by-layer deposition of the medium patterned according to a three-dimensional (3D) projection and formation of the biogel may be done by subjecting the medium to the energy source (e.g., a laser). For example, the laser may be projected along a light path in accordance to the 3D projection in order to polymerize the polymer precursors in the medium and form at least a portion of the 3D matrix comprising the plurality of cells and the biogel. In another aspect, the method may comprise a manual layer-by-layer deposition of the medium using a pipette or a capillary tube to deposit at least one microdroplet of the medium onto a substrate. In this example, a 3D projection comprising the pattern to be printed may not be necessary, rather the microdroplets of the medium may be subjected to an energy source (e.g., a heat or light source) once deposited, in order to form at least a portion of the 3D matrix comprising the biogel and the plurality of cells. In yet another aspect, the method may comprise a layer-by-layer deposition of the medium by use of a microfluidic device. The microfluidic device may control total volume of a microdroplet of the medium that is deposited in a layer-by-layer manner onto a substrate. The microfluidic device may control total number of cells per each microdroplet of the medium that is deposited in a layer-by-layer manner onto a substrate. In yet another aspect, the method may comprise a layer-by-layer deposition of the medium by use of a printer. The printer may be a laser printer, a layer-by-layer inkjet printer (e.g., a thermal inkjet printer or a piezoelectric inkjet printer), a layer-by-layer extrusion 3D printer (e.g., a pneumatic extrusion bioprinter or a mechanical extrusion bioprinter), or any combination thereof. Microdroplets of medium may be combined with other microdroplets such that cells may be organized into functional multi-cellular tissue niches.

Layered microdroplets may be cured, fused, solidified, gelled, crosslinked, polymerized, or photopolymerized in sequence or all at once using an energy source or via a chemical (e.g., a crosslinker or a photoinitiator). The energy source may be an energy beam, a heat source, or a light source. The energy source may be a laser, such as a fiber laser, a short-pulsed laser, or a femto-second pulsed laser. The energy source may be a heat source, such as a thermal plate, a lamp, an oven, a heated water bath, a cell culture incubator, a heat chamber, a furnace, a drying oven, or any combination thereof. The energy source may be a light source, such as white light, infrared light, ultraviolet (UV) light, near infrared (NIR) light, visible light, a light emitting diode (LED), or any combination thereof. The energy source may be a sound energy source, such as an ultrasound probe, a sonicator, an ultrasound bath, or any combination thereof. The energy source may be an electromagnetic radiation source, such as a microwave source, or any combination thereof.

The medium may be physically polymerized in order to form a biogel. The medium may be polymerized by a heat source in order to form a biogel. The medium may be chemically polymerized in order to form a biogel; for example, by use of a cross-linker. Non-limiting examples of cross-linkers include 1-ethyl-3-(3-dimethylaminopropyl)-carbodiimide (EDC), glutaraldehyde, and 1-ethyl-3-3-dimethyl aminopropyl carbodiimide (EDAC). The medium may comprise a photoinitiator, a cross-linker, collagen, hyaluronic acid and other glycosaminoglycans, poly-dl-lactic-co-glycolic acid (PLGA), poly-1-lactic acid (PLLA), polyglycolic acid (PGA), alginate, gelatin, agar, or any combination thereof. The biogel may comprise a photoinitiator, a cross-linker, collagen, hyaluronic acid and other glycosaminoglycans, poly-dl-lactic-co-glycolic acid (PLGA), poly-1-lactic acid (PLLA), polyglycolic acid (PGA), alginate, gelatin, agar, or any combination thereof. The polymer precursor may be collagen, hyaluronic acid and other glycosaminoglycans, poly-dl-lactic-co-glycolic acid (PLGA), poly-1-lactic acid (PLLA), polyglycolic acid (PGA), alginate, gelatin, agar, or any combination thereof.

The biogel may be a hydrogel. The biogel may be a biocompatible hydrogel. The biogel may be a polymeric hydrogel. The biogel may be a hydrogel bead. The biogel may be a hydrogel nanoparticle. The biogel may be a hydrogel droplet. The biogel may be a hydrogel microdroplet.

The microdroplet may have a diameter measuring at least about 10 microns (μm) to about 1000 μm. The microdroplet may have a diameter measuring at least about 10 μm. The microdroplet may have a diameter measuring at most about 1,000 μm. The microdroplet may have a diameter measuring about 10 μm to about 50 μm, about 10 μm to about 100 μm, about 10 μm to about 200 μm, about 10 μm to about 300 μm, about 10 μm to about 400 μm, about 10 μm to about 500 μm, about 10 μm to about 600 μm, about 10 μm to about 700 μm, about 10 μm to about 800 μm, about 10 μm to about 900 μm, about 10 μm to about 1,000 μm, about 50 μm to about 100 μm, about 50 μm to about 200 μm, about 50 μm to about 300 μm, about 50 μm to about 400 μm, about 50 μm to about 500 μm, about 50 μm to about 600 μm, about 50 μm to about 700 μm, about 50 μm to about 800 μm, about 50 μm to about 900 μm, about 50 μm to about 1,000 μm, about 100 μm to about 200 μm, about 100 μm to about 300 μm, about 100 μm to about 400 μm, about 100 μm to about 500 μm, about 100 μm to about 600 μm, about 100 μm to about 700 μm, about 100 μm to about 800 μm, about 100 μm to about 900 μm, about 100 μm to about 1,000 μm, about 200 μm to about 300 μm, about 200 μm to about 400 μm, about 200 μm to about 500 μm, about 200 μm to about 600 μm, about 200 μm to about 700 μm, about 200 μm to about 800 μm, about 200 μm to about 900 μm, about 200 μm to about 1,000 μm, about 300 μm to about 400 μm, about 300 μm to about 500 μm, about 300 μm to about 600 μm, about 300 μm to about 700 μm, about 300 μm to about 800 μm, about 300 μm to about 900 μm, about 300 μm to about 1,000 μm, about 400 μm to about 500 μm, about 400 μm to about 600 μm, about 400 μm to about 700 μm, about 400 μm to about 800 μm, about 400 μm to about 900 μm, about 400 μm to about 1,000 μm, about 500 μm to about 600 μm, about 500 μm to about 700 μm, about 500 μm to about 800 μm, about 500 μm to about 900 μm, about 500 μm to about 1,000 μm, about 600 μm to about 700 μm, about 600 μm to about 800 μm, about 600 μm to about 900 μm, about 600 μm to about 1,000 μm, about 700 μm to about 800 μm, about 700 μm to about 900 μm, about 700 μm to about 1,000 μm, about 800 μm to about 900 μm, about 800 μm to about 1,000 μm, or about 900 μm to about 1,000 μm. The microdroplet may have a diameter measuring about 10 μm, about 50 μm, about 100 μm, about 200 μm, about 300 μm, about 400 μm, about 500 μm, about 600 μm, about 700 μm, about 800 μm, about 900 μm, or about 1,000 μm.

The microdroplet may have a volume of about 1 microliter (μl) to about 500 μl. The microdroplet may have a volume of at least about 1 μl. The microdroplet may have a volume of at most about 500 μl. The microdroplet may have a volume of about 1 μl to about 2 μl, about 1 μl to about 3 μl, about 1 μl to about 4 μl, about 1 μl to about 5 μl, about 1 μl to about 10 μl, about 1 μl to about 20 μl, about 1 μl to about 25 μl, about 1 μl to about 50 μl, about 1 μl to about 75 μl, about 1 μl to about 100 μl, about 1 μl to about 500 μl, about 2 μl to about 3 μl, about 2 μl to about 4 μl, about 2 μl to about 5 μl, about 2 μl to about 10 μl, about 2 μl to about 20 μl, about 2 μl to about 25 μl, about 2 μl to about 50 μl, about 2 μl to about 75 μl, about 2 μl to about 100 μl, about 2 μl to about 500 μl, about 3 μl to about 4 μl, about 3 μl to about 5 μl, about 3 μl to about 10 μl, about 3 μl to about 20 μl, about 3 μl to about 25 μl, about 3 μl to about 50 μl, about 3 μl to about 75 μl, about 3 μl to about 100 μl, about 3 μl to about 500 μl, about 4 μl to about 5 μl, about 4 μl to about 10 μl, about 4 μl to about 20 μl, about 4 μl to about 25 μl, about 4 μl to about 50 μl, about 4 μl to about 75 μl, about 4 μl to about 100 μl, about 4 μl to about 500 μl, about 5 μl to about 10 μl, about 5 μl to about 20 μl, about 5 μl to about 25 μl, about 5 μl to about 50 μl, about 5 μl to about 75 μl, about 5 μl to about 100 μl, about 5 μl to about 500 μl, about 10 μl to about 20 μl, about 10 μl to about 25 μl, about 10 μl to about 50 μl, about 10 μl to about 75 μl, about 10 μl to about 100 μl, about 10 μl to about 500 μl, about 20 μl to about 25 μl, about 20 μl to about 50 μl, about 20 μl to about 75 μl, about 20 μl to about 100 μl, about 20 μl to about 500 μl, about 25 μl to about 50 μl, about 25 μl to about 75 μl, about 25 μl to about 100 μl, about 25 μl to about 500 μl, about 50 μl to about 75 μl, about 50 μl to about 100 μl, about 50 μl to about 500 μl, about 75 μl to about 100 μl, about 75 μl to about 500 μl, or about 100 μL to about 500 μl. The microdroplet may have a volume of about 1 μl, about 2 μl, about 3 μl, about 4 μl, about 5 μl, about 10 μl, about 20 μl, about 25 μl, about 50 μl, about 75 μl, about 100 μl, or about 500 μl.

The biogel may be a solution with a viscosity ranging from at least about 1×10⁻³ Pascal-second (Pa·s) to about 100,000 Pa·s or more when measured at about 25 degrees Celsius (° C.). When measured at about 25 degrees Celsius (° C.), the biogel may have a viscosity of about 0.001 Pa·s to about 100,000 Pa·s. When measured at about 25 degrees Celsius (° C.), the biogel may have a viscosity of at least about 0.001 Pa·s. When measured at about 25 degrees Celsius (° C.), the biogel may have a viscosity of at most about 100,000 Pa·s. When measured at about 25 degrees Celsius (° C.), the biogel may have a viscosity of about 0.001 Pa·s to about 0.01 Pa·s, about 0.001 Pa·s to about 0.1 Pa·s, about 0.001 Pa·s to about 1 Pa·s, about 0.001 Pa·s to about 10 Pa·s, about 0.001 Pa·s to about 100 Pa·s, about 0.001 Pa·s to about 1,000 Pa·s, about 0.001 Pa·s to about 10,000 Pa·s, about 0.001 Pa·s to about 50,000 Pa·s, about 0.001 Pa·s to about 100,000 Pa·s, about 0.01 Pas to about 0.1 Pa·s, about 0.01 Pa·s to about 1 Pa·s, about 0.01 Pas to about 10 Pa·s, about 0.01 Pas to about 100 Pa·s, about 0.01 Pa·s to about 1,000 Pa·s, about 0.01 Pa·s to about 10,000 Pa·s, about 0.01 Pa·s to about 50,000 Pa·s, about 0.01 Pa·s to about 100,000 Pa·s, about 0.1 Pa·s to about 1 Pa·s, about 0.1 Pa·s to about 10 Pa·s, about 0.1 Pa·s to about 100 Pa·s, about 0.1 Pa·s to about 1,000 Pa·s, about 0.1 Pas to about 10,000 Pa·s, about 0.1 Pa·s to about 50,000 Pa·s, about 0.1 Pa·s to about 100,000 Pa·s, about 1 Pa·s to about 10 Pa·s, about 1 Pa·s to about 100 Pa·s, about 1 Pa·s to about 1,000 Pa·s, about 1 Pa·s to about 10,000 Pa·s, about 1 Pa·s to about 50,000 Pa·s, about 1 Pa·s to about 100,000 Pa·s, about 10 Pa·s to about 100 Pa·s, about 10 Pa·s to about 1,000 Pa·s, about 10 Pa·s to about 10,000 Pa·s, about 10 Pa·s to about 50,000 Pa·s, about 10 Pa·s to about 100,000 Pa·s, about 100 Pa·s to about 1,000 Pa·s, about 100 Pa·s to about 10,000 Pa·s, about 100 Pa·s to about 50,000 Pa·s, about 100 Pa·s to about 100,000 Pa·s, about 1,000 Pa·s to about 10,000 Pa·s, about 1,000 Pa·s to about 50,000 Pa·s, about 1,000 Pa·s to about 100,000 Pa·s, about 10,000 Pa·s to about 50,000 Pa·s, about 10,000 Pa·s to about 100,000 Pa·s, or about 50,000 Pa·s to about 100,000 Pa·s. When measured at about 25 degrees Celsius (° C.), the biogel may have a viscosity of about 0.001 Pa·s, about 0.01 Pa·s, about 0.1 Pa·s, about 1 Pa·s, about 10 Pa·s, about 100 Pa·s, about 1,000 Pa·s, about 10,000 Pa·s, about 50,000 Pa·s, or about 100,000 Pa·s.

The biogel may be a hydrogel comprising a plurality of cells. The biogel may be a hydrogel comprising a plurality of non-hydrogel beads. The biogel may be a hydrogel comprising a plurality of non-hydrogel nanoparticles. The biogel may be a hydrogel comprising a plurality of non-hydrogel microparticles. The biogel may be a hydrogel comprising a plurality of non-hydrogel nanorods. The biogel may be a hydrogel comprising a plurality of non-hydrogel nanoshells. The biogel may be a hydrogel comprising a plurality of liposomes. The biogel may be a hydrogel comprising a plurality of non-hydrogel nanowires. The biogel may be a hydrogel comprising a plurality of non-hydrogel nanotubes. The biogel may be a gel in which the liquid component is water. A biogel may be a network of polymer chains in which water is the dispersion medium. The network of polymer chains maybe a network of hydrophilic polymer chains. The network of polymer chains maybe a network of hydrophobic polymer chains. The biogel may be a degradable hydrogel. The biogel may be a non-degradable hydrogel. The biogel may be a resorbable hydrogel. The biogel may be a hydrogel comprising naturally-derived polymers such as collagen.

### Additional Applications

In some examples, the methods and systems provided herein are used to print three-dimensional (3D) non-biological structures. The 3D non-biological structure may be a “smart” filter. The 3D non-biological structure may be a bioreactor. The 3D non-biological structure may be a biofilter. As used herein, the term “non-biological structure” refers to a structure that does not contain living cells.

Surface area to volume changes can speed or slow a chemical reaction with larger surface area to volume ratios increasing the speed, lower surface area to volume ratios decreasing the speed. Increased surface area to volume ratios are used in devices such as catalytic converters and bioreactors for cell growth. Cell growth is often dependent on efficient distribution of oxygen and nutrients while wastes are removed. Therefore, three-dimensional multi-channeled systems, as described herein, provide improved efficiency for cell growth.

Reproducible, thin-walled materials of complex or multi-layered systems are difficult to create with consistency. Our process can produce layers of thin materials in a repeatable manner with numerous applications in smart filter, bio-filtration, and bioreactor devices designed for cell differentiation and or expansion. Smart filtration or bio-filtration systems benefit significantly from high surface area to volume ratios and can improve chemical processes such as osmosis, chemical separation, and chemical sequestration. Similarly, in biological processes such as cell growth and development highly porous and, or high surface-area to volume ratio structures that allow for gas and nutrient exchange promote cell growth and development.

Deposition of materials using three-dimensional lithography or holographic lithography improves consistency and surface area to volume ratios of small capillary beds relative to surrounding surface areas. Holographic lithography can quickly produce structures with numerous channels of various sizes in a given material or complex channel systems that allow for alterations in flow rate and sheer forces that inform cell development and division. In addition, chemical separation and distribution may be facilitated by complex channel and capillary systems. This represents an improvement over industrial processes to build these filters are based on random materials deposition and organization such that there can be heterogeneity, and or lack of control over small features that, when repeated improve performance of filtration, separation, or systems that require chemical reactions to occur.

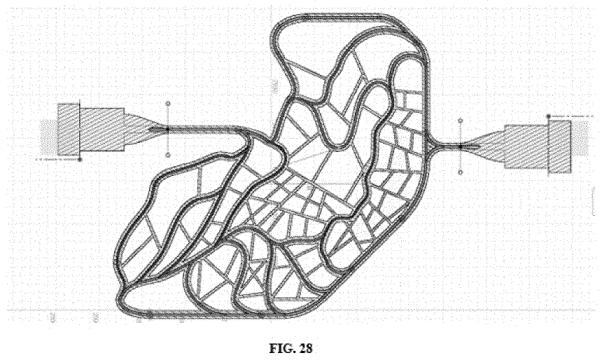

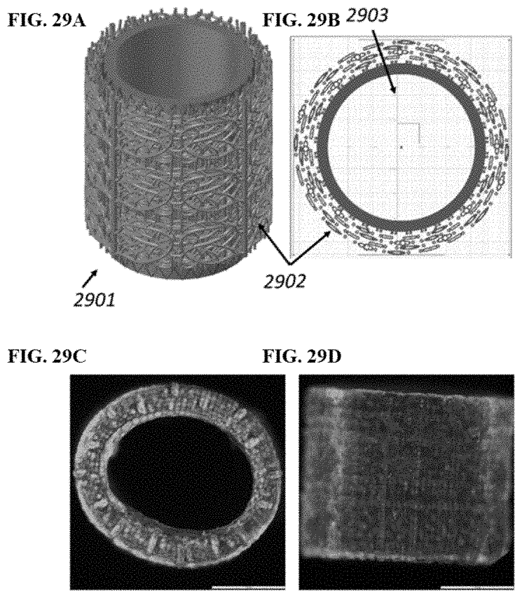

FIGS. 29A-29D show a blood vessel printed using methods and systems described herein. FIG. 29A shows an example of 3D model of a blood vessel 2901. The blood vessel 2901 may have supporting microvasculature 2902. The blood vessel 2901 may be printed at least partially encapsulating one or more cells. The blood vessel may have one or more cells added to it after printing. The one or more cells may be configured to be cultured to form an operable blood vessel. FIG. 29B shows a cross section of blood vessel 2901. The blood vessel may have an interior space 2903. The interior space may have a diameter of at least about 1, 5, 10, 15, 20, 25, 30, 40, 50, 60, 70, 80, 90, 100, 150, 200, 250, 300, 400, 500, 600, 700, 800, 900, 1,000, 1,500, 2,000, 3,000, 4,000, 5,000, 6,000, 7,000, 8,000, 9,000, or more microns. The interior space may have a diameter of at most about 9,000, 8,000, 7,000, 6,000, 5,000, 4,000, 3,000, 2,000, 1,500, 1,000, 900, 800, 700, 600, 500, 400, 300, 200, 150, 100, 90, 80, 70, 60, 50, 40, 30, 25, 20, 15, 10, 5, 1, or less microns. The blood vessel may have an interior diameter in a range defined by two proceeding values. For example, the blood vessel can have a diameter from 75 to 150 microns.

FIGS. 29C and 29D show a printed and cultured blood vessel. The contrast in the images may be caused by fluorescence intensity of von Willebrand factor stained cells. The scale bars may be 1 millimeter.

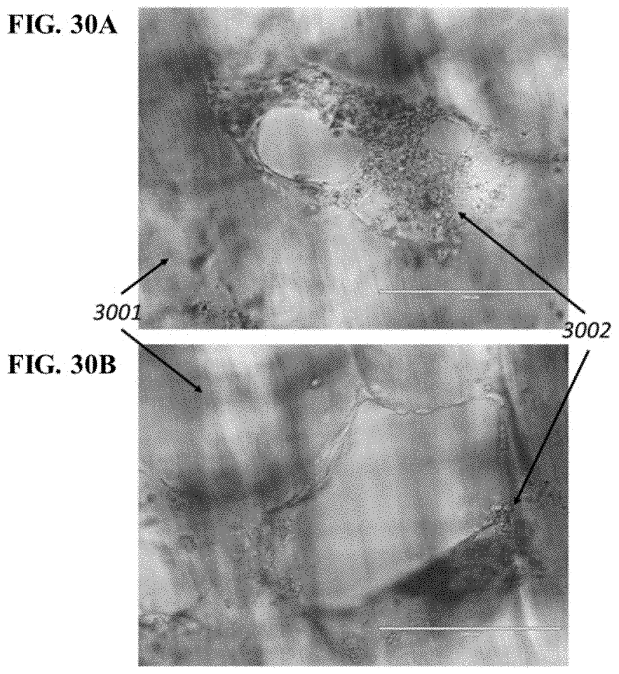

FIGS. 30A-30B show that cells growing on vasculature structures. The scale bars may be 200 microns. The vasculature structure 3001 may comprise agents to promote cell growth as described elsewhere herein. The vasculature structure may provide a substrate for growth of one or more cells 3002. The one or more cells may be cells configured to provide the functionality of native vasculature within a subject. For example, arterial cells can be harvested from a subject, cultured on the vasculature structure and the resultant blood vessel can be positioned within a subject.

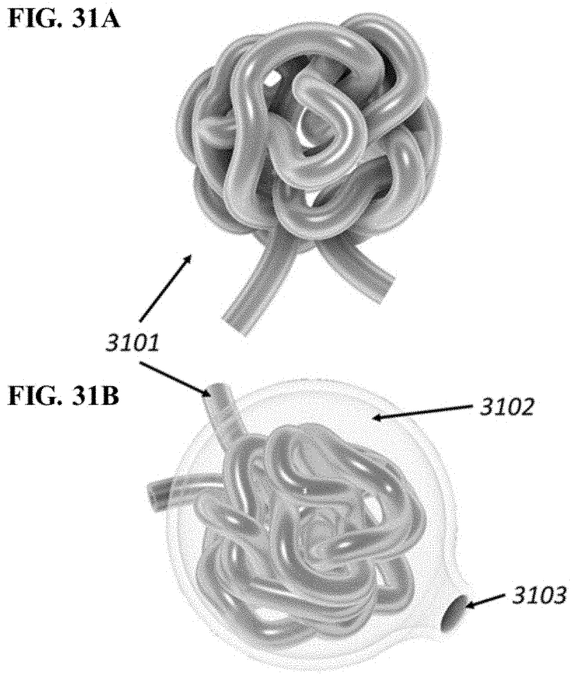

FIG. 31A shows a glomerular capillary knot. The glomerular capillary knot 3101 may have a capillary wall thickness of at least about 1, 2, 3, 4, 5, 6, 7, 8, 9, 10, 15, 20, 25, 30, 45, 50, 75, 100, or more microns. The glomerular capillary knot may have an inner diameter of at least about 1, 2, 3, 4, 5, 6, 7, 8, 9, 10, 15, 20, 25, 30, 45, 50, 75, 100, or more microns. The glomerular capillary knot may be configured to provide a plasma filtration rate similar to that of a mammal, such as human, pig, dog, monkey, rat, mouse nephron plasma filtration rate. FIG. 31B shows an example glomerular capillary knot surrounded by a Bowman's capsule. The glomerular knot 3101 may be surrounded by a Bowman's capsule 3102. The Bowman's capsule 3102 may be configured such that a distance between the inner walls of the Bowman's capsule and the glomerular capillary knot allows for cell growth and filtrate flow into the proximal tube of the kidney nephron through outlet 3103.

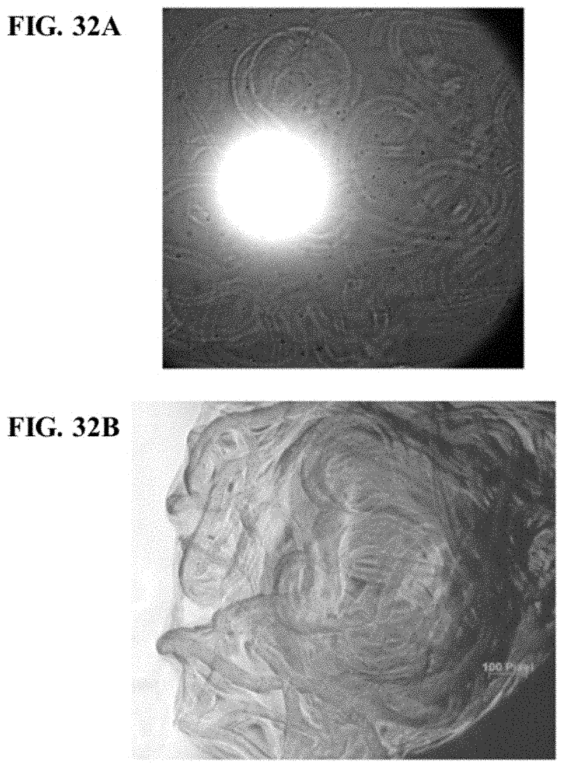

FIGS. 32A-32B show a proximal tube and glomerulus. FIG. 32A shows an example of a proximal tube and glomerulus during a printing process. The proximal tube and glomerulus may be printed using methods and system described elsewhere herein. FIG. 32B shows an example of a glomerulus and proximal tube after printing.

### Subtractive Printing Methods

In an aspect, the present disclosure provides methods and systems used to generate 3D structures comprising subtractive printing. In some examples, the subtractive printing is laser ablation. In some aspects, the subtractive printing is 3D holographic laser ablation.

Targeted laser-based ablation of materials is a useful manufacturing process. Channels, pores, holes and tubes can be built in a solid material using the process of laser ablation. Three-dimensional holographic laser ablation has numerous advantages over conventional methods, including increased speed and resolution of the ablation process.

### Methods and Systems for Generating Three Dimensional Models

The present disclosure provides methods for generating a three-dimensional (3D) structure corresponding to a biological material comprising a subunit having a surface for performing a biological function. A method for generating a three-dimensional (3D) structure corresponding to a biological material comprising a subunit having a surface for performing a biological function, may comprise (a) using at least a number of vessels coupled to the subunit over the surface to generate a computer model of the 3D structure comprising the subunit and the vessels; and (b) using the computer model from (a) to print the 3D structure, which 3D structure is implantable in a body of a subject.

The present disclosure also provides methods for generating a three-dimensional (3D) structure corresponding to a biological material comprising a subunit having a surface for performing a biological function. A method for generating a three-dimensional (3D) structure corresponding to a biological material comprising a subunit having a surface for performing a biological function may comprise using at least a number of vessels coupled to the subunit over the surface to generate a superunit comprising the subunit and the vessels in computer memory; and using one or more computer processors to combine the superunit generated in (a) with one or more other superunits to generate a computer model of the 3D structure corresponding to the biological material.

The present disclosure also provides methods for generating a three-dimensional (3D) structure corresponding to a biological material comprising a subunit having a surface for performing a biological function. A three-dimensional (3D) structure corresponding to a biological material comprising a subunit having a surface for performing a biological function may comprise using at least a number of vessels coupled to said subunit over said surface to generate a computer model of said 3D structure comprising said subunit and said vessels; and using one or more computer processors to transmit said 3D structure to a printer to print said 3D structure according to said computer model from (a), wherein said 3D structure is implantable in a body of a subject.

The 3D structure may be printed using methods and systems described elsewhere herein based at least in part on a 3D model. The 3D model may comprise features of the 3D structure. For example, a 3D model of a kidney can have glomerulus subunits combined with other vasculature to form nephron superunits that can be combined to form a 3D model of a kidney. The 3D structure may correspond to the 3D model. For example, adding a plurality of capillaries to the 3D model can add the same capillaries to the 3D structure when it is printed.

The biological material may be a biological material found in an animal or a human. The biological material may be an organ or organoid as described elsewhere herein (e.g., a kidney, a lung, a pancreas, a thyroid). The biological material may comprise blood vessels. The subunits of the biological material may be named subunits (e.g., an alveolus, a glomerulus, a glomerulus with a Bowman's capsule around the glomerulus) or other subunits (e.g., a volume of a tissue that does not have a recognized name). The subunits may be of an organ or an organoid. Identification of one or more subunits may be determined by factors comprising capillary location, capillary density, lymphatic location, lymphatic density, association of one or more cells into a barrier (e.g., a wall of an artery), and the like. For example, finding a concentration of capillaries or other vessels in a kidney can indicate the presence of a nephron functional unit. The generating a computer model may further comprise using at least in part a generalized location of the vessels (e.g., capillaries) coupling to the subunit (e.g., glomerulus), the walls of the subunit (e.g., a Bowman's capsule), or both to identify the surface. The determining may comprise using at least in part a plurality of 3D estimations derived from a diameter approximation of the subunit or comparing a volume calculation of the 3D structure to a predetermined range of volumes of the biological material to determine the surface area. For example, the capillaries of a glomerulus may be destroyed in the process of preparing histological samples, so an estimation of the size of the glomerulus capillary knot can be generated using the void left behind in the histology sample. In another example, a computer based calculation of the total surface area of the lungs divided by the number of alveoli, taking into account the efficacy of each alveolus at exchanging gasses, can give an estimation of the active surface area of alveoli in the lungs. In another example, a series of different volumes of glomerulus can be screened against the total size of a kidney and a needed filtration rate, and the ideal volume can be selected based in part on those criteria. The vessel may be a capillary, and the method may further comprise determining a length of the capillary comprising using an oxygen exchange rate between the capillary's volume of blood and the subunit, where the subunit couples to the capillary. For example, knowing the size of an alveolus and that capillaries can exchange 0.2 mL of air over a 1 mm length, a length of capillary can be determined to provide a desired exchange volume.

The biological function may be at least a part of an overall function of an organ. The function may be an exchange of gasses. For example, providing a gas diffusion membrane is a function of alveoli, which is a part of the overall function of a lung. The gasses may be oxygen, nitrogen, carbon dioxide, and the like.

In some case, the function may be an exchange of metabolically active compounds. For example, a kidney uses a length of capillary blood vessels to remove waste from the blood stream for concentration and disposal. The metabolically active compounds may comprise nutrients, sugars, salts, amino acids, waste compounds, and the like. The nutrients may be vitamins, proteins, fats, minerals, and the like. The sugars may be monosaccharides (e.g., glucose, fructose), disaccharides (e.g., lactose, sucrose), or polysaccharides. The salts may be nutrient salts, mineral salts, waste salts, or the like. The amino acids may be naturally occurring amino acids (e.g., arginine, tyrosine) or non-natural amino acids. The waste compounds may be compounds generated by the subject (e.g., uric acid) or compounds from an external source (e.g., bacteria, toxins). The function may be a filtration of plasma. For example, a nephron can filter waste and toxins out of blood plasma.

The function may be generating one or more biologically relevant materials.

Biologically relevant materials may comprise hormones, antibodies, immunological proteins, and the like. The function may be a function of an organ. For example, the determination of the function of a lung can comprise properties such as the tidal volume of the lung. The method may comprise using at least in part a plurality of 3D estimations derived from a diameter approximation of the subunit, comparing a volume calculation of the 3D structure to a predetermined range of volumes of the biological material, and the like. The volume may comprise a tidal volume of air in a lung. The volume may comprise a residual volume of air in a lung.

The subunit may have a property related to the biological function of the subunit and such property may be used to determine a surface area of a subunit. The property of the one or more subunits of the biological material may relate to the function of the subunit. For example, the ability of a nephron to exchange nutrients and waste in blood can depend on the surface area of capillaries within a glomerulus within the nephron. In this example, the surface area is a property of the nephron subunit. The property may be a surface area, a density of cells, a density of functional components, and the like. The property may comprise a plurality of properties. The property may be used to determine a surface area of the subunit. For example, the total capillary surface area of a glomerulus can be determined by determining the amount of nutrient and waste exchange needed and dividing that by the nutrient and waste exchanged per unit surface area of the capillary. In another example, the surface area of an alveolus and its associated capillaries can be determined by finding the amount of gas exchange needed and dividing it by the gas exchange for a given unit area.

The property may be determined by measuring the property from a sample (e.g., a histology slide), using one or more computer processors to generate one or more parameters (e.g., a computer model generating a capillary density) or one or more estimations of the parameters, or a combination thereof. The one or more parameters may be filtrate volumes, ranges of structural sizes, packing densities, gross volume measurements, and the like. Filtrate volumes may be volumes required to filter an amount of a material out of a liquid (e.g., waste salts from blood). Ranges of structural sizes may be a range within which the subunit fits. Ranges of structural size may be at least about 10 nm, 100 nm, 1 μm, 10 μm, 25 μm, 50 μm, 75 μm, 100 μm, 150 μm, 200 μm, 250 μm, 300 μm, 400 μm, 500 μm, 600 μm, 700 μm, 800 μm, 900 μm, 1 mm, 5 mm, 10 mm, or more. Ranges of structural size may be at most about 10 mm, 5 mm, 1 mm, 900 μm, 800 μm, 700 μm, 600 μm, 500 μm, 400 μm, 300 μm, 250 μm, 200 μm, 150 μm, 100 μm, 75 μm, 50 μm, 25 μm, 10 μm, 1 μm, 100 nm, 10 nm, or less. Ranges of structural size may be a range from any two proceeding values. For example, a range of structural size may be from 10 μm to 500 μm. The packing densities may be a density of at least one feature. Kidney structures that may have a structural size as described above may be glomerular capillary wall thickness, glomerular capillary inner diameter, glomerular capsule diameter, glomerular capsule wall thickness, glomerular capillary length, convoluted proximal tubule length, convoluted proximal tubule diameter, straight proximal tubule length, straight proximal tubule diameter, loop of Henle length, loop of Henle diameter, straight distal tubule length, straight distal tubule diameter, convoluted distal tube length, convoluted distal tubule diameter, and the like. The packing densities may be at least about 1 feature per 1 μm³, per 10 μm³, per 50 μm³, per 75 μm³, per 100 μm³, per 150 μm³, per 200 μm³, per 250 μm³, per 500 μm³, per 750 μm³, per 1,000 μm³, per 2,500 μm³, per 5,000 μm³, per 10,000 μm³, per 100,000 μm³, or more. The packing densities may be at most about 1 feature per 100,000 μm³, 10,000 μm³, per 5,000 μm³, per 2,500 μm³, per 1,000 μm³, per 750 μm³, per 500 μm³, per 250 μm³, per 200 μm³, per 150 μm³, per 100 μm³, per 75 μm³, per 50 μm³, per 10 μm³, per 1 μm³, or less. For example, a packing density of a 10 μm³ nephron can be 1 nephron per 10 μm³. Gross volume measurements may be measurements of a volume of an existing tissue. For example, the gross volume of a lung of a patient can be found by imaging the lung and extracting a volume based on the image.

The vessels may comprise one or more blood vessels. The vessels may comprise one or more lymphatic vessels. The blood vessels may comprise one or more capillary blood vessels. The blood vessels may comprise capillary blood vessels and larger blood vessels. For example, a 3D model corresponding to an arterial blood vessel can be formed with capillary blood vessels surrounding the arterial blood vessel. The blood vessels may be of a length up to the length of the 3D model. For example, a blood vessel can run the entire length of a 3D model corresponding to a kidney. The blood vessels may be configured to provide a similar oxygen exchange rate between the blood vessels and the surrounding tissue as blood vessels in a biological sample. For example, if a human thyroid has blood vessels that provide about 0.1 L/hr of oxygen to the thyroid tissue, the blood vessels in the 3D structure can be configured to also provide about 0.1 L/hr to the 3D structure.

The blood vessels may be placed on a subunit. For example, a series of capillaries can be placed to cover an alveolus. The blood vessels may be placed such that the blood vessels have a similar volume to a volume in a biological sample. For example, the coverage of a human alveolus in capillaries can be measured, and the 3D structure can have a number of capillaries that fill a volume that is equal to the capillaries on the human alveolus. The blood vessels may be configured to produce a similar function to blood vessels in the biological material.

The blood vessels may be configured to allow a subunit to share a blood flow with at least one other subunit. The sharing of blood flow may be through a larger blood vessel. For example, two adjacent nephrons can use one large blood vessel to supply blood to the capillaries of both nephrons. The vessel may be a capillary. The number of vessels coupling to the subunit may be determined by using a total surface area of a plurality of capillaries placed within a space. For example, the number of capillaries to be placed on a subunit can be determined by defining the surface area of the subunit and determining how many capillaries can fit in or around that surface area or around the space surrounding the surface area. In another example, the number of capillaries may be determined by determining the number of capillaries that will provide a certain level of oxygen to the subunit. The method may comprise determining a length of a vessel (e.g., a blood vessel, a lymphatic vessel). The method may comprise determining the length of a capillary using an oxygen exchange rate between the capillary's volume of blood and the subunit coupled to the capillary.

The superunit may be a combination of at least about 2, 3, 4, 5, 6, 7, 8, 9, 10, 15, 25, 50, 100, 1,000, 10,000, 100,000, 1,000,000, or more subunits. The superunit may be generated by computing a packing density for a plurality of subunits. For example, the packing density of a subunit can be computed based on the shape and volume of the subunit, and a superunit can be formed that maximizes the packing of the subunits into a given space. The superunit may be generated by comping another parameter (e.g., exposure of the plurality of subunits to a membrane). For example, an alveolus can be combined with other alveoli to form an alveolar sac. In this example, the alveoli can be combined to maximize exposure to air.

The computer model of superunit may be combined with at least about 2, 3, 4, 5, 6, 7, 8, 9, 10, 15, 25, 50, 100, 1,000, 10,000, 100,000, 1,000,000, or more other computer models of superunits to generate a computer model of the 3D structure corresponding the biological material. A single superunit may generate a computer model of the 3D structure. The 3D structure may approximate the biological material. For example, 1.2 million nephrons can be combined to generate a structure corresponding to a kidney. The 3D structure may be configured to maintain a blood pressure within and/or outside the 3D structure. The maintenance of blood pressure may be important for making the 3D structure compatible with being implanted within a subject. The 3D structure may correspond to the biological material. The 3D structure may have structure derived from a structure found within a human. The 3D structure may be derived from images of a structure within a human, measurements of a structure outside a human, or a combination thereof. For example, measurements from histopathological slides can be combined with MRI images to form a 3D structure corresponding to a thyroid.

The combining of a superunit with the one or more additional superunits may comprise 3D space packing estimations based at least in part on a size of the superunits. The 3D space packing estimations may be based at least in part on known physiologic requirements. The known physiologic requirements may be physiologic requirements such as an exchange rate of gasses, filtration of plasma per unit volume, or other requirements that relate to the function of the biological material.

The 3D structure may comprise lymphatic vessels. The lymphatic vessels may comprise one or more drainage points. The drainage points may be a plurality of drainage points. The drainage points may be configured to function similarly to lymphatic drainage systems. The drainage points may be connected to a larger lymphatic system. The larger lymphatic system may be configured to be placed in a subject and attached to the lymphatic system of the subject. The drainage points may be configured to facilitate passive return of fluid that has leaked out of another blood vessel. The drainage points may be distributed at least in part based on the number of capillaries in the 3D structure. For example, an area with three capillaries can have one drainage point configured to return the blood leaked from the three capillaries. In another example, more drainage points can be placed in areas with more capillaries. The method may further comprise using the one or more processors to add a plurality of drainage points to the computer model. The drainage points may be distributed at least in part based on a system of generative design or a generative design algorithm. The system of generative design may avoid capillaries. The 3D structure may be configured to maintain tissue circulatory homeostasis. The system of generative 3D design may be configured to provide a high enough density of drainage points to maintain tissue homeostasis. The plurality of drainage points may be configured to maintain a net positive fluid pressure of the biological material or 3D structure. The drainage points may be placed in the 3D structure based at least in part on a capillary density. The capillary density may be a capillary density in developed tissue structures. The drainage points may be placed in the 3D structure by a generative design algorithm. The generative design algorithm may use a combination of one or more physiological parameters (e.g., parameters measured from a sample) and/or one or more generated parameters (e.g., parameters generated by a fluid simulation computer program). The drainage points may be distributed at least in part based on a blood pressure of the 3D structure. For example, the blood pressure across the 3D structure can be calculated, and more drainage points can be placed in areas that are expected to have a higher blood pressure.

The 3D structure with the plurality of drainage points may be output as a file format suitable for 3D printing. The file format may be a .stl file, a .obj file, a .vrml file, a .ply file, a .fbx file, or the like. The 3D structure with the plurality of drainage points may be printed using a 3D printer as described elsewhere herein. For example, the 3D structure with the plurality of drainage points can be printed by generating a 3D holographic array of points in a medium.

A plurality of cells may be cultured and/or in the 3D structure. The culturing of cells may generate an object with similar function as an organ. For example, a 3D structure corresponding to a kidney may be cultured with cells from a subject to form a replacement kidney for the subject. The cells may be at least partially encapsulated in the 3D structure.

The 3D structure may be printed according to the generated 3D model described herein. The printing may be using a 3D printer as described elsewhere herein. The 3D printer may be a light-based 3D printer. The 3D structure may be printed by (a) providing a media chamber comprising a medium comprising (i) a plurality of cells and (ii) one or more polymer precursors; and (b) directing at least one energy beam to the medium in the media chamber along at least one energy beam path that is patterned into a three-dimensional (3D) projection in accordance with the computer model for printing the 3D structure in computer memory, to form at least a portion of the 3D structure comprising (i) at least a subset of the plurality of cells, and (ii) a polymer formed from the one or more polymer precursors.

The plurality of cells may be selected form the group consisting of stromal endothelial cells, endothelial cells, follicular reticular cells or precursors thereof, epithelial cells, mesangial cells, kidney glomerulus parietal cells, kidney glomerulus podocytes, kidney proximal tubule brush border cells, Loop of Henle thing segment cells, thick ascending limb cells, kidney distal tubule cells, collecting duct principal cells, collecting duct intercalated cells, interstitial kidney cells, cuboidal cells, columnar cells, alveolar type I cells, alveolar type II cells, alveolar macrophages, and pneumocytes. The plurality of cells may be of one or more cell types. The plurality of cells may be of a subject. For example, a biopsy of a kidney can be taken, cells extracted from the biopsy, the cells added to the media chamber, a structure of a new kidney printed, and the cells cultured to generate a new kidney.

The present disclosure provides systems for generating a three-dimensional (3D) structure corresponding to a biological material comprising a subunit having a surface for performing a biological function. A system for generating a three-dimensional (3D) structure corresponding to a biological material comprising a subunit having a surface for performing a biological function, may comprise one or more computer processors that are individually or collectively programmed to (a) use at least a number of vessels coupled to said subunit over said surface to generate a computer model of said 3D structure comprising said subunit and said vessels; and (b) transmit said computer model from (a) to a 3D printer for printing said 3D structure, which 3D structure is implantable in a body of a subject.

The present disclosure provides systems for generating a three-dimensional (3D) structure corresponding to a biological material comprising a subunit having a surface for performing a biological function. A system for generating a three-dimensional (3D) structure corresponding to a biological material comprising a subunit having a surface for performing a biological function, may comprise one or more computer processors that are individually or collectively programmed to (a) use at least a number of vessels coupled to said subunit over said surface to generate a superunit comprising said subunit and said vessels in computer memory; and (b) combine said superunit generated in (a) with one or more other superunits to generate a computer model of said 3D structure corresponding to said biological material

### Computer Systems

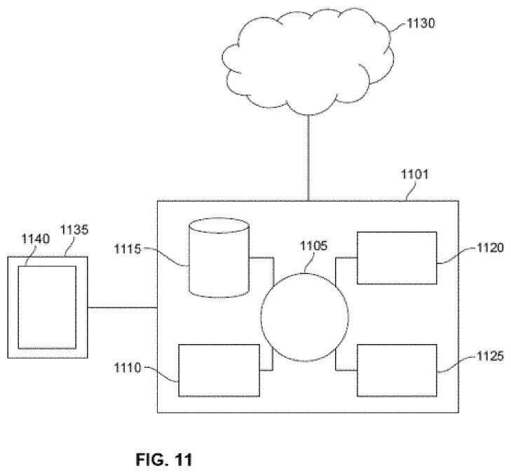

The present disclosure provides computer systems that are programmed to implement methods of the disclosure. FIG. 11 shows a computer system 1101 that is programmed or otherwise configured to receive a computer model of the 3D lymphoid organoid and/or 3D cell-containing matrix in computer memory; generate a point-cloud representation or lines-based representation of the computer model of the 3D lymphoid organoid and/or 3D cell-containing matrix in computer memory; and direct the at least one energy source to direct the energy beam to the medium in the media chamber along at least one energy beam path in accordance with the computer model of the 3D lymphoid organoid and/or 3D cell-containing matrix, and to subject at least a portion of the polymer precursors to form at least a portion of the 3D lymphoid organoid and/or 3D cell-containing matrix. The computer system 1101 can also be programmed or otherwise configured to generate a 3D structure corresponding to a biological material. The computer system 1101 can regulate various aspects of computer model generation and design (including the generation of 3D structures corresponding to a biological material containing a plurality of blood and lymphatic vessels), image generation, holographic projection, and light modulation of the present disclosure, such as, for example, receiving or generating a computer-aided-design (CAD) model of a desired three-dimensional (3D) biological material structure to be printed, such as a 3D lymphoid organoid and/or a 3D cell-containing matrix. The computer system 1101 can convert the CAD model or any other type of computer model such as a point-cloud model or a lines-based model into an image of the desired 3D lymphoid organoid and/or 3D cell-containing matrix to be printed. The computer system 1101 can project the image of the desired 3D lymphoid organoid and/or 3D cell-containing matrix holographically. The computer system 1101 can modulate a light source, an energy source, or an energy beam such that a light path or an energy beam path is created by the computer system 1101. The computer system 1101 can direct the light source, the energy source, or the energy beam along the light path or the energy beam path. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), cloud based computing services (e.g., Amazon Web Services), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, status of the printing process (e.g., displaying an illustration of the 3D lymphoid organoid and/or 3D cell-containing matrix representing the 3D tissue portions printed prior to completion of the process), manual controls of the energy beams (e.g., emergency stop buttons controlling the on/off states of the energy beam), and display indicators designed to e.g., display a remote oxygen concentration, a carbon dioxide concentration, a humidity measurement, and/or a temperature measurement within the media chamber. Examples of UI's include, without limitation, a graphical user interface (GUI) and web-based user interface.

### EXAMPLES

The following examples are provided for illustrative purposes. These examples are not intended to be limiting.

### Example 1—Kidney Nephron

In an example, a kidney nephron is printed using the methods and systems disclosed herein. First, a library of antibodies is produced in printed lymph node organoids. Three dimensional kidney nephron model, as shown in FIGS. 16A-16C, is a multi-part structure to filter blood and create urine. The close capillary association with the proximal tubule structure, the descending loop of Henle, the ascending loop of Henle, and the distal tubule allows for reabsorption and salt balancing through cells and/or osmotic forces. The nephron structure may have a short or long loop of Henle. Capillaries may be diffuse or dense surrounding the nephron structure. An inlet and outlet is illustrated in FIGS. 16A-C for a single nephron. Multiple nephrons may be stacked in bundles and associated with the same inlet, and outlet blood vessels, and collecting ducts for urine. The loop of Henle may be up to about 8 cm in length and as short as about 1 cm in length. The entire structure may be elongated such that no folds exist.

### Example 2—Glomerular Capillaries

In another example, glomerular capillaries are printed using the methods and systems disclosed herein. FIG. 16D shows glomerular capillaries and capsule with proximal tubule with capillary bed wrapped around it. The glomerulus illustrated in FIG. 16D has 6 capillaries that are in contact with the glomerular capsule. The capsule may have more or fewer capillaries. The capillaries may or may not be touching the wall of the capsule. The capillary bed is shown as a minimal set of capillaries that may be expanded such that are larger blood volume surrounds the proximal tubules. Proximal tubules may be convoluted (as shown in FIG. 16D) straight or in a network. The tubules may be encapsulated in a net of capillaries.

### Example 3—Tube with Bio-Printed Thin Interface

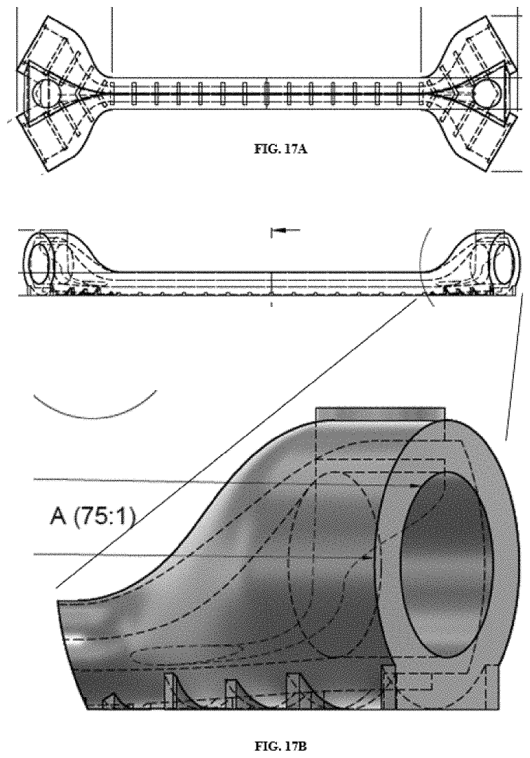

In an example, a tube comprising channels with a bio-printed thin interface is printed using the methods and systems disclosed herein. FIGS. 17-19 illustrate various views of example tubes comprising two channels. The material between two channels of biological materials may be as thin as about 5 micrometers (μm) and as large as about 1 millimeter (mm). FIGS. 17A-17C show a tube comprising a distance of about 30 microns (μm) in between a first channel and a second channel. The length of the interface (e.g., the channel) may be as long as about 2 mm and as short as about 50 micrometers, wherein the length is defined as the distance between a first end and a second end of the channel. This tube comprising channels allow for thin cell-wall thickness interfaces that match biological structures. These facilitate the study of cell-cell interactions across a membrane and, or study of gas or materials exchange. Microfluidic tubing may be affixed into the structure in a chip format or the structure may be embedded in a material such as polydimethylsiloxane (PDMS) and tubes affixed from top of the cured structure, as shown in FIG. 19. This is unique as biological materials with a thin interface that allow for flow and study of cells that require flow can be studied. Tubes may be open as pictured or sealed before embedding in a different material for microfluidics support.

The unique feature of these tubes is that ultra-fine (micron-scale) walls between tubes can be created out of biological materials for the query of cell-cell interactions across barriers that are representative of human physiology; for example, blood-brain barrier, kidney nephron barriers, liver-blood barrier, or a tissue of any type barrier with blood.

### Example 4—Channel Injection Ports

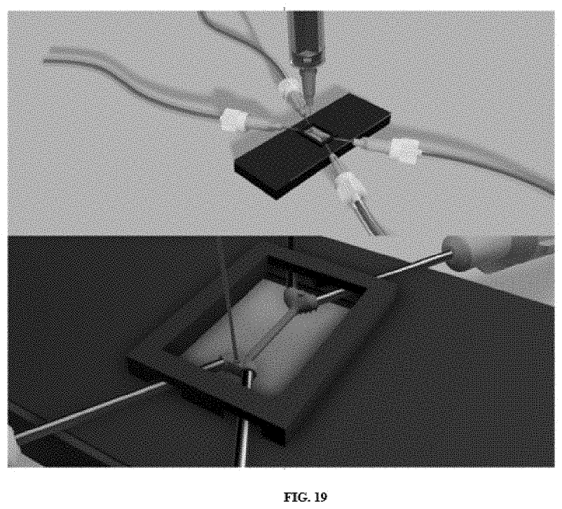

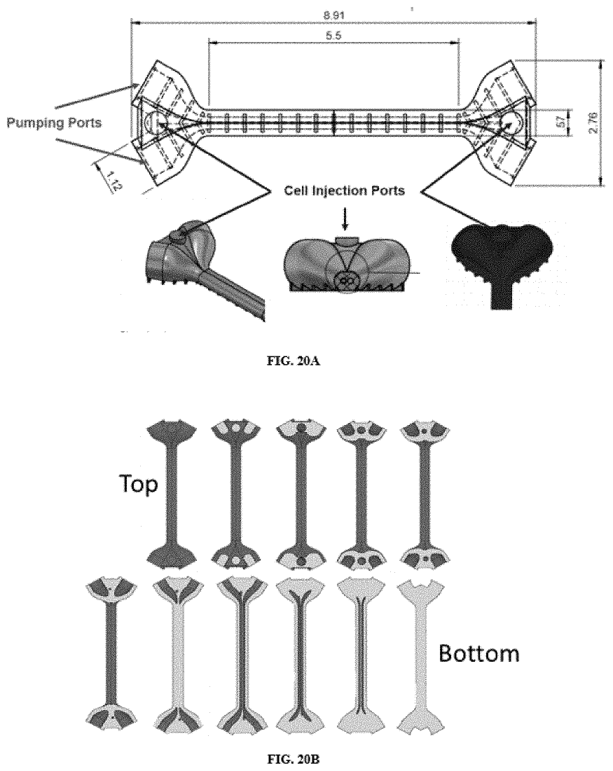

In another example, a tube comprising channels and injection ports is printed using the methods and systems disclosed herein. FIG. 20A shows a tube comprising channel injection ports. The channel injection ports can be used for introduction of cells on the top, center of the structure can be utilized to introduce cells, compounds or other materials and flush them through the system prior to introduction of flow. The injection ports that are independent of flow are on the each end of the structure. Injection ports independent of the flow system can be introduced in numerous places in the structure to allow for cell seeding, injection of materials such as collagen or gelatin coatings, and/or injection of cell growth factors.

FIG. 20B shows a representative structure depicting two channels with two injection ports and with a top-down or a z-axis slicing demonstrating the hollow portions of the tubes and channel connection.

FIG. 21 illustrates example configurations of tubule arrays. Tubules may be convoluted or straight and the system may contain 1, 2, 3, 4, or up to 8 tubules in an array. End on view of 3, 4 or 8 tubules. These tubes may share conjoining walls. Tubes may be of varied diameters and wall thicknesses. FIGS. 22A-22C illustrates an example tubule unit.

### Example 5—Larger Lung Structures: Numerous Alveolar Spaces Conjoined with Shared Capillary System

In another example, an alveolar structure with a shared capillary system is printed using the methods and systems described herein. FIGS. 23A-23B illustrate example alveolar structures. Alveoli can be joined into larger structures to form functional units of gas exchange across a thin membrane. Gas exchange is facilitated by thin walls of biological material or cells contained in a printed extracellular membrane space that is interspersed with a capillary system of blood or liquid containing tubes that both surround and are found inside of alveolar spaces. This system increases the surface area to volume ratio of gas exchange. Numerous units of alveoli together for alveolar sacs which in turn can be linked together through larger airways and vascular systems to form lung lobes. For ease of visualization blood flow is represented as being on different sides of the airway, however arteriole and venous systems may be next to each other and sharing a wall. Engineered capillary sizes may range from about 5 micrometers to about 50 micrometers in diameter with walls ranging from about 0.5 micron to about 30 micron in thickness.

### Example 6—Printed Alveolar Structures

In another example, an alveolar structure was printed using the methods and systems described herein. FIGS. 24A-24C show example designs of alveolar structures. FIG. 24D shows an image of a printed alveolar structure with a diameter of about 400 microns, with capillaries surrounding it, and independent channels for flow. The printed alveolar structure had distinguishing features; for example, the structure was nearly clear and may fluoresce faintly with an excitation of a ultraviolet light at a wavelength of about 500 nanometers (nm). The alveolar outlets were about 80 micron in diameter. The printed alveolar structure tolerated drying out and re-hydrating well.

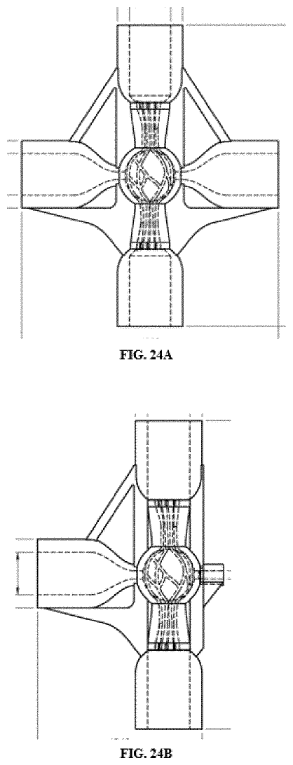

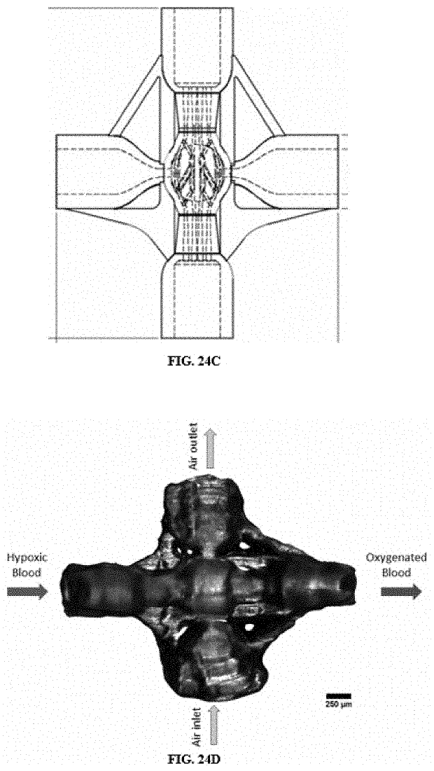

FIGS. 25A and 25B show add-on structures comprising printed capillaries. FIG. 25A shows closely associated (cardiac tissue spacing), 250 micron long capillaries that were printed in under 6 minutes. The capillaries were printed with FDA-approved biocompatible, bioresorbable materials. FIG. 25B shows a fluorescence microscopy image of printed capillaries during a positive pressure flow test with a 5 μm fluorescent particles.

### Example 7—Printed Basket Structures

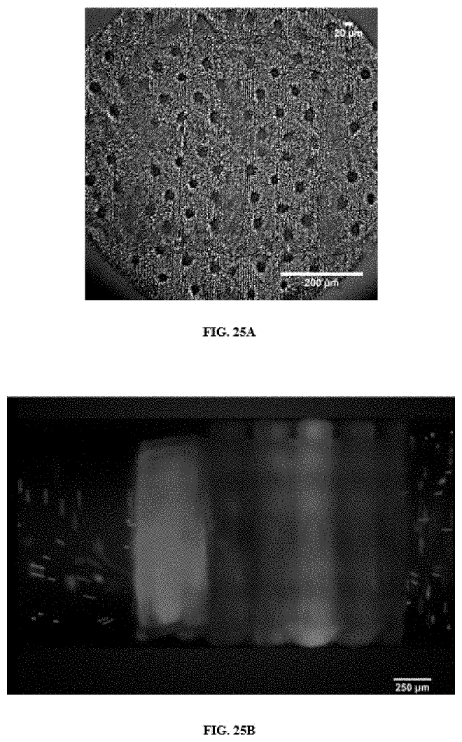

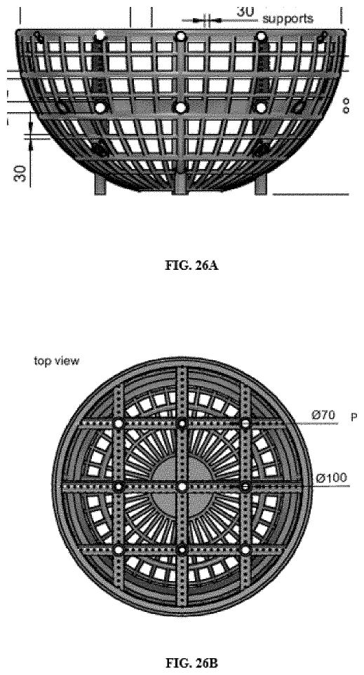

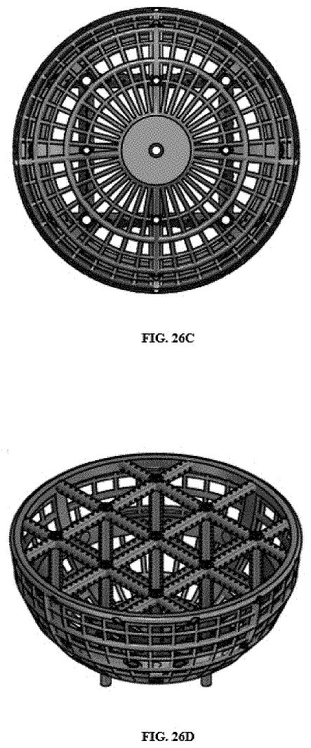

In another example, a basket structure was printed using the methods and systems described herein. FIGS. 26A-26D show various views of an example design of a basket structure. FIGS. 27A-27B show brightfield and fluorescence microscopy images of the printed basket structure. As shown in FIGS. 27A-27B, the printed basket structures successfully reproduced the designs shown in FIGS. 26A-26D.

### Example 8—Capillary Bed Structure

In another example, a capillary bed structure is printed using the methods and systems provided herein. FIG. 28 shows a capillary bed design comprising a first port and a second port. The capillary bed may have a convoluted design of tubes and/or channels. The capillary bed may have an interconnected network of tubes and/or channels.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 9—Generating a 3D Structure Corresponding to a Kidney

In another example, a 3D structure corresponding to a kidney was printed using methods and systems described herein. The histopathological samples were analyzed for the location of the active components of the kidney, the nephrons, by looking at properties such as the location of capillary blood vessels, lymphatics, and the like. Further, the histopathological samples of human kidneys were taken and measured to determine estimations of properties such as the length of various components of the nephron (e.g., the straight proximal tubule length, the loop of Helene length). The measured estimations were combined with gross measurements of the kidney (e.g., total volume, filtering capacity) to further refine the estimations of the properties of the nephrons. For example, the glomerular capillary wall can be about 10 μm, the glomerular capillary inner diameter can be about 22 μm, the glomerular inner capsule diameter can be about 400 μm, the glomerular capsule wall can be about 50 μm thick, the glomerulus capillary can have a volume of about 5.14 μm′, and the glomerulus capillaries can have a length of about 13.53 mm. Additionally, the convoluted proximal tubule can be about 16.9 mm long, the convoluted proximal tubule can be about 63 μm in outer diameter and about 33 μm in inner diameter, the straight proximal tubule can be about 1.1 mm long the straight proximal tubule can have an inner and outer diameter of about 63 μm and about 33 μm respectively, the loop of Henle thin A and D limbs can be about 3.18 mm long, the loop of Henle thin A and D limbs can have about 40 μm outer diameter and a about 20 μm inner diameter, the straight distal tubule can be about 2.45 mm long, the straight distal tubule can have about a 55 outer diameter and about a 25 inner diameter, the convoluted distal tubule can be about 2.5 mm long, and the convoluted distal tubule can have an outer diameter of about 55 μm and an inner diameter of about 25 μm.

For the glomerulus, the portion of the kidney where filtration occurs, the observed volume of the glomerulus combined with the known physiologic filtrate exchange rate was used to determine the volume of capillary blood vessels to be placed in the glomerulus. The length of the capillaries was determined by the minimal length of capillary calculated to produce a desired amount of oxygen delivery to the tissue.

Once the properties of an individual nephron are determined, a plurality of nephrons were combined together. The nephrons were combined by taking into account blood delivery to the individual nephrons, such that a number of nephrons can use the same arterial blood delivery and venous return. Once the nephrons were combined in this way, there was an established arterial delivery and venous return system from the group of nephrons. The total size of the desired kidney was then determined (e.g., by measuring a kidney in a subject), and a blood vessel super-structure scheme is developed. The blood vessel super-structure was based on the vasculature of human kidneys. The group of nephrons was then queried for its ability to fit within the predetermined blood vessel super-structure. The combined nephron-blood vessel super-structure was designed to maintain a similar blood pressure to a human kidney, reducing the risk of complications due to intolerance to blood pressure fluctuations. The process of placing the groups of nephrons within the super-structure continued until enough nephrons have been added to replicate the functionality of a human kidney, typically over 1,000,000 nephrons in total.

With the nephrons in place within the super-structure, lymphatic system structure analogues were added to the super-structure to provide passive return of blood plasma that has leaked through the walls of the capillaries. The ends of the lymphatic analogues were of similar size to the capillaries in the kidney. The lymphatic analogues were distributed based at least in part on the local concentration of capillaries and expected regional blood pressures. The lymphatic analogues can be distributed and branched in part based on a generative 3D modeling program that places the analogues to avoid the capillaries and nephron structures already in the super-structure while ensuring enough lymphatic analogues to maintain circulatory homeostasis. Not including the lymphatic analogues can result in accumulation of fluids in the kidney, leading to possible rejection of the kidney or even subject death.

1.-87. (canceled)

88. A method for generating a three-dimensional (3D) structure corresponding to a biological material comprising a subunit having a surface for performing a biological function, comprising:
(a) using at least a number of vessels coupled to said subunit over said surface to generate a superunit comprising said subunit and said vessels in computer memory; and
(b) using one or more computer processors to combine said superunit generated in (a) with one or more other superunits to generate a computer model of said 3D structure corresponding to said biological material.

89. The method of claim 88, wherein said biological material is a kidney, said biological function comprises an exchange of a plurality of metabolically active compounds, and said subunit is a glomerulus.

90. The method of claim 89, wherein said plurality of metabolically active compounds are selected from the group consisting of nutrients, sugars, salts, amino acids, and metabolic wastes.

91. The method of claim 88, wherein said biological material is a lung, said biological function comprises an exchange of gasses, and said subunit is an alveolus.

92. The method of claim 88, wherein said biological function comprises a filtration of plasma.

93. The method of claim 88, wherein said vessels comprise one or more blood vessels and one or more lymphatic vessels.

94. The method of claim 93, wherein said one or more blood vessels comprise one or more capillaries.

95. The method of claim 88, further comprising using said one or more processors to add a plurality of drainage points to said computer model from (a).

96. The method of claim 95, wherein said plurality of drainage points is configured to maintain a net positive fluid pressure within said biological material.

97. The method of claim 95, wherein said plurality of drainage points are placed based at least in part by a generative design algorithm.

98. The method of claim 95, wherein said plurality of drainage points are placed based at least in part on a density of a plurality of capillaries.

99. The method of claim 95, wherein said plurality of drainage points are placed based at least in part on a blood pressure of said 3D structure.

100. The method of claim 88, further comprising using at least in part a generalized location of said vessels coupling to said subunit, walls of said subunit, or both to identify said surface.

101. The method of claim 88, further comprising determining a surface area of said subunit having said surface.

102. The method of claim 101, wherein determining comprises using at least in part a plurality of three-dimensional estimations derived from a diameter approximation of said subunit or comparing a volume calculation of said 3D structure to a predetermined range of volumes of said biological material to determine said surface area.

103. The method of claim 88, when said vessel is a capillary, further comprising using a total surface area of a plurality of capillaries placed within a space to determine said number of vessels.

104. The method of claim 88, when said vessel is a capillary, further comprising determining a length of said capillary comprising using an oxygen exchange rate between said capillary's volume of biological fluid and said subunit, wherein said subunit couples to said capillary.

105. The method of claim 88, wherein said 3D structure is configured to maintain tissue circulatory homeostasis.

106. The method of claim 88, wherein said 3D structure is printed by:
(a) providing a media chamber comprising a medium comprising one or more polymer precursors; and
(b) directing at least one energy beam to said medium in said media chamber along at least one energy beam path that is patterned into a three-dimensional (3D) projection in accordance with said computer model for printing said 3D structure in computer memory, to form at least a portion of said 3D structure comprising a polymer formed from said one or more polymer precursors.

107. The method of claim 106, wherein said medium further comprises a plurality of cells.
